SEQUENCE LISTING (48 sequences)

SEQ ID NO: 1 - protein, 26 aa, Artificial Sequence
  Synthetic Peptide of Formula (I)
  VARIANT (2)..(2): X1 in Formula (I). Xaa is selected from D-Leu, D-Ile, and D-Nle
  VARIANT (3)..(3): X2 in Formula (I). Xaa is selected from L-Dab, L-Dap, D-Dab, D-Dap, D-Orn, D-Lys, D-Ala, and D-Arg
  VARIANT (5)..(6): X3 and X4 in Formula (I). Xaa is selected from D-Leu, D-Ile, and D-Nle
  VARIANT (7)..(7): X5 in Formula (I). Xaa is selected from L-Dab, L-Dap, D-Dab, D-Dap, D-Orn, D-Lys, D-Ala, and D-Arg
  VARIANT (9)..(9): X6 in Formula (I). Xaa is selected from D-Leu, D-Ile, and D-Nle
  VARIANT (11)..(11): X7 in Formula (I). Xaa is selected from L-Dab, L-Dap, D-Dab, D-Dap, D-Orn, D-Lys, D-Ala, and D-Arg
  VARIANT (13)..(13): X8 in Formula (I). Xaa is selected from L-Dab, L-Dap, D-Dab, D-Dap, and D-Lys
  VARIANT (14)..(15): X9 and X10 in Formula (I). Xaa is selected from D-Lys, L-Dab, L-Dap, D-Dab, D-Dap, and D-Ala
  VARIANT (16)..(16): X11 in Formula (I). Xaa is selected from L-Dab, L-Dap, D-Dab, D-Dap, and D-Lys
  VARIANT (17)..(17): X12 in Formula (I). Xaa is selected from D-Leu, D-Ile, and D-Nle
  VARIANT (18)..(18): X13 in Formula (I). Xaa is selected from L-Dab, L-Dap, D-Dab, D-Dap, D-Orn, D-Lys, D-Ala, and D-Arg
  VARIANT (20)..(21): X14 and X15 in Formula (I). Xaa is selected from  D-Leu, D-Ile, and D-Nle
  VARIANT (22)..(22): X16 in Formula (I). Xaa is selected from L-Dab, L-Dap, D-Dab, D-Dap, D-Orn, D-Lys, D-Ala, and D-Arg
  VARIANT (24)..(24): X17 in Formula (I). Xaa is selected from D-Leu, D-Ile, and D-Nle
  VARIANT (26)..(26): X18 in Formula (I). Xaa is selected from L-Dab, L-Dap, D-Dab, D-Dap, D-Cys, D-Ser, D-Orn, D-Lys, and D-Arg; all other amino acids are D-amino acids
KXXSXXXTXSXAXXXXXXTXXXAXSX

SEQ ID NO: 2 - protein, 26 aa, Artificial Sequence
  Synthetic Peptide
  MISC_FEATURE (3)..(3): Xaa is L-2,4-Diaminobutyric acid
  MISC_FEATURE (7)..(7): Xaa is L-2,4-Diaminobutyric acid
  MISC_FEATURE (11)..(11): Xaa is L-2,4-Diaminobutyric acid
  MISC_FEATURE (18)..(18): Xaa is L-2,4-Diaminobutyric acid
  MISC_FEATURE (22)..(22): Xaa is L-2,4-Diaminobutyric acid
  MISC_FEATURE (26)..(26): Xaa is L-2,4-Diaminobutyric acid; all other amino acids are D-amino acids
KLXSLLXTLSXAKAAKLXTLLXALSX

SEQ ID NO: 3 - protein, 26 aa, Artificial Sequence
  Synthetic Peptide
  MISC_FEATURE (3)..(3): Xaa is L-2,4-Diaminobutyric acid
  MISC_FEATURE (7)..(7): Xaa is L-2,4-Diaminobutyric acid
  MISC_FEATURE (11)..(11): Xaa is L-2,4-Diaminobutyric acid
  MISC_FEATURE (18)..(18): Xaa is L-2,4-Diaminobutyric acid
  MISC_FEATURE (22)..(22): Xaa is L-2,4-Diaminobutyric acid; all other amino acids are D-amino acids
KLXSLLXTLSXAKAAKLXTLLXALSC

SEQ ID NO: 4 - protein, 26 aa, Artificial Sequence
  Synthetic Peptide
  MISC_FEATURE (3)..(3): Xaa is L-2,3-Diaminopropionic acid
  MISC_FEATURE (7)..(7): Xaa is L-2,3-Diaminopropionic acid
  MISC_FEATURE (11)..(11): Xaa is L-2,3-Diaminopropionic acid
  MISC_FEATURE (18)..(18): Xaa is L-2,3-Diaminopropionic acid
  MISC_FEATURE (22)..(22): Xaa is L-2,3-Diaminopropionic acid; all other amino acids are D-amino acids
KLXSLLXTLSXAKAAKLXTLLXALSC

SEQ ID NO: 5 - protein, 26 aa, Artificial Sequence
  Synthetic Peptide
  MOD_RES (2)..(2): Xaa is D-Nle
  MISC_FEATURE (3)..(3): Xaa is L-2,4-Diaminobutyric acid
  MOD_RES (5)..(6): Xaa is D-Nle
  MISC_FEATURE (7)..(7): Xaa is L-2,4-Diaminobutyric acid
  MOD_RES (9)..(9): Xaa is D-Nle
  MISC_FEATURE (11)..(11): Xaa is L-2,4-Diaminobutyric acid
  MOD_RES (17)..(17): Xaa is D-Nle
  MISC_FEATURE (18)..(18): Xaa is L-2,4-Diaminobutyric acid
  MOD_RES (20)..(21): Xaa is D-Nle
  MISC_FEATURE (22)..(22): Xaa is L-2,4-Diaminobutyric acid
  MOD_RES (24)..(24): Xaa is D-Nle; all other amino acids are D-amino acids
KXXSXXXTXSXAKAAKXXTXXXAXSS

SEQ ID NO: 6 - protein, 26 aa, Artificial Sequence
  Synthetic Peptide
  MOD_RES (2)..(2): Xaa is D-Nle
  MISC_FEATURE (3)..(3): Xaa is L-2,3-Diaminopropionic acid
  MOD_RES (5)..(6): Xaa is D-Nle
  MISC_FEATURE (7)..(7): Xaa is L-2,3-Diaminopropionic acid
  MOD_RES (9)..(9): Xaa is D-Nle
  MISC_FEATURE (11)..(11): Xaa is L-2,3-Diaminopropionic acid
  MOD_RES (17)..(17): Xaa is D-Nle
  MISC_FEATURE (18)..(18): Xaa is L-2,3-Diaminopropionic acid
  MOD_RES (20)..(21): Xaa is D-Nle
  MISC_FEATURE (22)..(22): Xaa is L-2,3-Diaminopropionic acid
  MOD_RES (24)..(24): Xaa is D-Nle; all other amino acids are D-amino acids
KXXSXXXTXSXAKAAKXXTXXXAXSS

SEQ ID NO: 7 - protein, 26 aa, Artificial Sequence
  Synthetic Peptide
  MISC_FEATURE (3)..(3): Xaa is L-2,4-Diaminobutyric acid
  MISC_FEATURE (7)..(7): Xaa is L-2,4-Diaminobutyric acid
  MISC_FEATURE (11)..(11): Xaa is L-2,4-Diaminobutyric acid
  MISC_FEATURE (18)..(18): Xaa is L-2,4-Diaminobutyric acid
  MISC_FEATURE (22)..(22): Xaa is L-2,4-Diaminobutyric acid; all other amino acids are D-amino acids
KLXSLLXTLSXAKAAKLXTLLXALSS

SEQ ID NO: 8 - protein, 26 aa, Artificial Sequence
  Synthetic Peptide
  MISC_FEATURE (3)..(3): Xaa is L-2,4-Diaminobutyric acid
  MISC_FEATURE (7)..(7): Xaa is L-2,4-Diaminobutyric acid
  MISC_FEATURE (11)..(11): Xaa is L-2,4-Diaminobutyric acid
  MISC_FEATURE (18)..(18): Xaa is L-2,4-Diaminobutyric acid
  MISC_FEATURE (22)..(22): Xaa is L-2,4-Diaminobutyric acid; all other amino acids are D-amino acids
KIXSIIXTISXAKAAKIXTIIXAISS

SEQ ID NO: 9 - protein, 26 aa, Artificial Sequence
  Synthetic Peptide
  MOD_RES (2)..(2): Xaa is D-Nle
  MISC_FEATURE (3)..(3): Xaa is L-2,4-Diaminobutyric acid
  MOD_RES (5)..(6): Xaa is D-Nle
  MISC_FEATURE (7)..(7): Xaa is L-2,4-Diaminobutyric acid
  MOD_RES (9)..(9): Xaa is D-Nle
  MISC_FEATURE (14)..(15): Xaa is L-2,4-Diaminobutyric acid
  MOD_RES (17)..(17): Xaa is Nle
  MOD_RES (20)..(21): Xaa is D-Nle
  MISC_FEATURE (22)..(22): Xaa is L-2,4-Diaminobutyric acid
  MOD_RES (24)..(24): Xaa is D-Nle; all other amino acids are D-amino acids
KXXSXXXTXSAAKXXKXATXXXAXSS

SEQ ID NO: 10 - protein, 26 aa, Artificial Sequence
  Synthetic Peptide
  MOD_RES (2)..(2): Xaa is D-Nle
  MISC_FEATURE (3)..(3): Xaa is L-2,3-Diaminopropionic acid
  MOD_RES (5)..(6): Xaa is D-Nle
  MISC_FEATURE (7)..(7): Xaa is L-2,3-Diaminopropionic acid
  MOD_RES (9)..(9): Xaa is D-Nle
  MISC_FEATURE (14)..(15): Xaa is L-2,3-Diaminopropionic acid
  MOD_RES (17)..(17): Xaa is D-Nle
  MOD_RES (20)..(21): Xaa is D-Nle
  MISC_FEATURE (22)..(22): Xaa is L-2,3-Diaminopropionic acid
  MOD_RES (24)..(24): Xaa is D-Nle; all other amino acids are D-amino acids
KXXSXXXTXSAAKXXKXATXXXAXSS

SEQ ID NO: 11 - protein, 26 aa, Artificial Sequence
  Synthetic Peptide
  MISC_FEATURE (3)..(3): Xaa is L-2,4-Diaminobutyric acid
  MISC_FEATURE (7)..(7): Xaa is L-2,4-Diaminobutyric acid
  MISC_FEATURE (14)..(15): Xaa is L-2,4-Diaminobutyric acid
  MISC_FEATURE (22)..(22): Xaa is L-2,4-Diaminobutyric acid; all other amino acids are D-amino acids
KLXSLLXTLSAAKXXKLATLLXALSS

SEQ ID NO: 12 - protein, 26 aa, Artificial Sequence
  Synthetic Peptide
  MISC_FEATURE (3)..(3): Xaa is L-2,4-Diaminobutyric acid
  MISC_FEATURE (7)..(7): Xaa is L-2,4-Diaminobutyric acid
  MISC_FEATURE (14)..(15): Xaa is L-2,4-Diaminobutyric acid
  MISC_FEATURE (22)..(22): Xaa is L-2,4-Diaminobutyric acid; all other amino acids are D-amino acids
KIXSIIXTISAAKXXKIATIIXAISS

SEQ ID NO: 13 - protein, 26 aa, Artificial Sequence
  Synthetic Peptide
  MOD_RES (2)..(2): Xaa is D-Nle
  MISC_FEATURE (3)..(3): Xaa is D-2,4-Diaminobutyric acid
  MOD_RES (5)..(6): Xaa is D-Nle
  MISC_FEATURE (7)..(7): Xaa is D-2,4-Diaminobutyric acid
  MOD_RES (9)..(9): Xaa is D-Nle
  MISC_FEATURE (14)..(15): Xaa is D-2,4-Diaminobutyric acid
  MOD_RES (17)..(17): Xaa is D-Nle
  MOD_RES (20)..(21): Xaa is D-Nle
  MISC_FEATURE (22)..(22): Xaa is D-2,4-Diaminobutyric acid
  MOD_RES (24)..(24): Xaa is D-Nle; all other amino acids are D-amino acids
KXXSXXXTXSAAKXXKXATXXXAXSS

SEQ ID NO: 14 - protein, 26 aa, Artificial Sequence
  Synthetic Peptide
  MOD_RES (2)..(2): Xaa is D-Nle
  MISC_FEATURE (3)..(3): Xaa is D-2,3-Diaminopropionic acid
  MOD_RES (5)..(6): Xaa is D-Nle
  MISC_FEATURE (7)..(7): Xaa is D-2,3-Diaminopropionic acid
  MOD_RES (9)..(9): Xaa is D-Nle
  MISC_FEATURE (14)..(15): Xaa is D-2,3-Diaminopropionic acid
  MOD_RES (17)..(17): Xaa is D-Nle
  MOD_RES (20)..(21): Xaa is D-Nle
  MISC_FEATURE (22)..(22): Xaa is D-2,3-Diaminopropionic acid
  MOD_RES (24)..(24): Xaa is D-Nle; all other amino acids are D-amino acids
KXXSXXXTXSAAKXXKXATXXXAXSS

SEQ ID NO: 15 - protein, 26 aa, Artificial Sequence
  Synthetic Peptide
  MISC_FEATURE (3)..(3): Xaa is D-2,4-Diaminobutyric acid
  MISC_FEATURE (7)..(7): Xaa is D-2,4-Diaminobutyric acid
  MISC_FEATURE (14)..(15): Xaa is D-2,4-Diaminobutyric acid
  MISC_FEATURE (22)..(22): Xaa is D-2,4-Diaminobutyric acid; all other amino acids are D-amino acids
KLXSLLXTLSAAKXXKLATLLXALSS

SEQ ID NO: 16 - protein, 26 aa, Artificial Sequence
  Synthetic Peptide
  MISC_FEATURE (3)..(3): Xaa is D-2,4-Diaminobutyric acid
  MISC_FEATURE (7)..(7): Xaa is D-2,4-Diaminobutyric acid
  MISC_FEATURE (14)..(15): Xaa is D-2,4-Diaminobutyric acid
  MISC_FEATURE (22)..(22): Xaa is D-2,4-Diaminobutyric acid; all other amino acids are D-amino acids
KIXSIIXTISAAKXXKIATIIXAISS

SEQ ID NO: 17 - protein, 26 aa, Artificial Sequence
  Synthetic Peptide
  MISC_FEATURE (3)..(3): Xaa is D-2,4-Diaminobutyric acid
  MISC_FEATURE (7)..(7): Xaa is D-2,4-Diaminobutyric acid
  MISC_FEATURE (11)..(11): Xaa is D-2,4-Diaminobutyric acid
  MISC_FEATURE (18)..(18): Xaa is D-2,4-Diaminobutyric acid
  MISC_FEATURE (22)..(22): Xaa is D-2,4-Diaminobutyric acid; all other amino acids are D-amino acids
KLXSLLXTLSXAKAAKLXTLLXALSS

SEQ ID NO: 18 - protein, 26 aa, Artificial Sequence
  Synthetic Peptide
  MISC_FEATURE (3)..(3): Xaa is L-2,4-Diaminobutyric acid
  MISC_FEATURE (7)..(7): Xaa is D-2,4-Diaminobutyric acid
  MISC_FEATURE (11)..(11): Xaa is D-2,4-Diaminobutyric acid
  MISC_FEATURE (18)..(18): Xaa is D-2,4-Diaminobutyric acid
  MISC_FEATURE (22)..(22): Xaa is L-2,4-Diaminobutyric acid; all other amino acids are D-amino acids
KLXSLLXTLSXAKAAKLXTLLXALSS

SEQ ID NO: 19 - protein, 26 aa, Artificial Sequence
  Synthetic Peptide
  MISC_FEATURE (3)..(3): Xaa is D-2,4-Diaminobutyric acid
  MISC_FEATURE (7)..(7): Xaa is L-2,4-Diaminobutyric acid
  MISC_FEATURE (11)..(11): Xaa is L-2,4-Diaminobutyric acid
  MISC_FEATURE (18)..(18): Xaa is L-2,4-Diaminobutyric acid
  MISC_FEATURE (22)..(22): Xaa is D-2,4-Diaminobutyric acid; all other amino acids are D-amino acids
KLXSLLXTLSXAKAAKLXTLLXALSS

SEQ ID NO: 20 - protein, 26 aa, Artificial Sequence
  Synthetic Peptide
  MISC_FEATURE (3)..(3): Xaa is D-2,4-Diaminobutyric acid
  MISC_FEATURE (7)..(7): Xaa is D-2,4-Diaminobutyric acid
  MISC_FEATURE (14)..(15): Xaa is D-2,4-Diaminobutyric acid
  MISC_FEATURE (22)..(22): Xaa is D-2,4-Diaminobutyric acid; all other amino acids are D-amino acids
KLXSLLXTLSAAKXXKLATLLXALSS

SEQ ID NO: 21 - protein, 42 aa, Artificial Sequence
  Synthetic Peptide
  MISC_FEATURE (3)..(3): Xaa is L-2,4-Diaminobutyric acid
  MISC_FEATURE (7)..(7): Xaa is L-2,4-Diaminobutyric acid
  MISC_FEATURE (14)..(15): Xaa is D-2,4-Diaminobutyric acid
  MISC_FEATURE (22)..(22): Xaa is L-2,4-Diaminobutyric acid; all other amino acids are D-amino acids
KLXSLLXTLSAAKXXKLATLLXALSSSYNTHETICPEPTIDE

SEQ ID NO: 22 - protein, 26 aa, Artificial Sequence
  Synthetic Peptide
  MISC_FEATURE (3)..(3): Xaa is D-2,4-Diaminobutyric acid
  MISC_FEATURE (7)..(7): Xaa is D-2,4-Diaminobutyric acid
  MISC_FEATURE (14)..(15): Xaa is L-2,4-Diaminobutyric acid
  MISC_FEATURE (22)..(22): Xaa is D-2,4-Diaminobutyric acid; all other amino acids are D-amino acids
KLXSLLXTLSAAKXXKLATLLXALSS

SEQ ID NO: 23 - protein, 26 aa, Artificial Sequence
  Synthetic Peptide
  MISC_FEATURE (3)..(3): Xaa is L-2,3-Diaminopropionic acid
  MISC_FEATURE (7)..(7): Xaa is L-2,3-Diaminopropionic acid
  MISC_FEATURE (11)..(11): Xaa is L-2,3-Diaminopropionic acid
  MISC_FEATURE (18)..(18): Xaa is L-2,3-Diaminopropionic acid
  MISC_FEATURE (22)..(22): Xaa is L-2,3-Diaminopropionic acid
  MISC_FEATURE (26)..(26): Xaa is L-2,3-Diaminopropionic acid; all other amino acids are D-amino acids
KLXSLLXTLSXAKAAKLXTLLXALSX

SEQ ID NO: 24 - protein, 26 aa, Artificial Sequence
  Synthetic Peptide
  MISC_FEATURE (3)..(3): Xaa is D-2,3-Diaminopropionic acid
  MISC_FEATURE (7)..(7): Xaa is D-2,3-Diaminopropionic acid
  MISC_FEATURE (11)..(11): Xaa is D-2,3-Diaminopropionic acid
  MISC_FEATURE (18)..(18): Xaa is D-2,3-Diaminopropionic acid
  MISC_FEATURE (22)..(22): Xaa is D-2,3-Diaminopropionic acid
  MISC_FEATURE (26)..(26): Xaa is D-2,3-Diaminopropionic acid; all other amino acids are D-amino acids
KLXSLLXTLSXAKAAKLXTLLXALSX

SEQ ID NO: 25 - protein, 26 aa, Artificial Sequence
  Synthetic Peptide
  MISC_FEATURE (3)..(3): Xaa is L-2,3-Diaminopropionic acid
  MISC_FEATURE (7)..(7): Xaa is L-2,3-Diaminopropionic acid
  MISC_FEATURE (11)..(11): Xaa is D-2,3-Diaminopropionic acid
  MISC_FEATURE (18)..(18): Xaa is D-2,3-Diaminopropionic acid
  MISC_FEATURE (22)..(22): Xaa is L-2,3-Diaminopropionic acid
  MISC_FEATURE (26)..(26): Xaa is L-2,3-Diaminopropionic acid; all other amino acids are D-amino acids
KLXSLLXTLSXAKAAKLXTLLXALSX

SEQ ID NO: 26 - protein, 26 aa, Artificial Sequence
  Synthetic Peptide
  MISC_FEATURE (3)..(3): Xaa is D-2,3-Diaminopropionic acid
  MISC_FEATURE (7)..(7): Xaa is D-2,3-Diaminopropionic acid
  MISC_FEATURE (11)..(11): Xaa is L-2,3-Diaminopropionic acid
  MISC_FEATURE (18)..(18): Xaa is L-2,3-Diaminopropionic acid
  MISC_FEATURE (22)..(22): Xaa is D-2,3-Diaminopropionic acid
  MISC_FEATURE (26)..(26): Xaa is D-2,3-Diaminopropionic acid; all other amino acids are D-amino acids
KLXSLLXTLSXAKAAKLXTLLXALSX

SEQ ID NO: 27 - protein, 26 aa, Artificial Sequence
  Synthetic Peptide
  MISC_FEATURE (3)..(3): Xaa is L-2,3-Diaminopropionic acid
  MISC_FEATURE (7)..(7): Xaa is L-2,3-Diaminopropionic acid
  MISC_FEATURE (11)..(11): Xaa is L-2,3-Diaminopropionic acid
  MISC_FEATURE (18)..(18): Xaa is L-2,3-Diaminopropionic acid
  MISC_FEATURE (22)..(22): Xaa is L-2,3-Diaminopropionic acid; all other amino acids are D-amino acids
KLXSLLXTLSXAKAAKLXTLLXALSS

SEQ ID NO: 28 - protein, 26 aa, Artificial Sequence
  Synthetic Peptide
  MISC_FEATURE (3)..(3): Xaa is D-2,3-Diaminopropionic acid
  MISC_FEATURE (7)..(7): Xaa is D-2,3-Diaminopropionic acid
  MISC_FEATURE (11)..(11): Xaa is D-2,3-Diaminopropionic acid
  MISC_FEATURE (18)..(18): Xaa is D-2,3-Diaminopropionic acid
  MISC_FEATURE (22)..(22): Xaa is D-2,3-Diaminopropionic acid; all other amino acids are D-amino acids
KLXSLLXTLSXAKAAKLXTLLXALSS

SEQ ID NO: 29 - protein, 26 aa, Artificial Sequence
  Synthetic Peptide
  MISC_FEATURE (3)..(3): Xaa is L-2,3-Diaminopropionic acid
  MISC_FEATURE (7)..(7): Xaa is L-2,3-Diaminopropionic acid
  MISC_FEATURE (14)..(15): Xaa is L-2,3-Diaminopropionic acid
  MISC_FEATURE (22)..(22): Xaa is L-2,3-Diaminopropionic acid
  MISC_FEATURE (26)..(26): Xaa is L-2,3-Diaminopropionic acid; all other amino acids are D-amino acids
KLXSLLXTLSAAKXXKLATLLXALSX

SEQ ID NO: 30 - protein, 26 aa, Artificial Sequence
  Synthetic Peptide
  MISC_FEATURE (3)..(3): Xaa is D-2,3-Diaminopropionic acid
  MISC_FEATURE (7)..(7): Xaa is D-2,3-Diaminopropionic acid
  MISC_FEATURE (14)..(15): Xaa is D-2,3-Diaminopropionic acid
  MISC_FEATURE (22)..(22): Xaa is D-2,3-Diaminopropionic acid
  MISC_FEATURE (26)..(26): Xaa is D-2,3-Diaminopropionic acid; all other amino acids are D-amino acids
KLXSLLXTLSAAKXXKLATLLXALSX

SEQ ID NO: 31 - protein, 26 aa, Artificial Sequence
  Synthetic Peptide
  MISC_FEATURE (3)..(3): Xaa is L-2,3-Diaminopropionic acid
  MISC_FEATURE (7)..(7): Xaa is L-2,3-Diaminopropionic acid
  MISC_FEATURE (14)..(15): Xaa is D-2,3-Diaminopropionic acid
  MISC_FEATURE (22)..(22): Xaa is L-2,3-Diaminopropionic acid
  MISC_FEATURE (26)..(26): Xaa is L-2,3-Diaminopropionic acid; all other amino acids are D-amino acids
KLXSLLXTLSAAKXXKLATLLXALSX

SEQ ID NO: 32 - protein, 26 aa, Artificial Sequence
  Synthetic Peptide
  MISC_FEATURE (3)..(3): Xaa is D-2,3-Diaminopropionic acid
  MISC_FEATURE (7)..(7): Xaa is D-2,3-Diaminopropionic acid
  MISC_FEATURE (14)..(15): Xaa is L-2,3-Diaminopropionic acid
  MISC_FEATURE (22)..(22): Xaa is D-2,3-Diaminopropionic acid
  MISC_FEATURE (26)..(26): Xaa is D-2,3-Diaminopropionic acid; all other amino acids are D-amino acids
KLXSLLXTLSAAKXXKLATLLXALSX

SEQ ID NO: 33 - protein, 26 aa, Artificial Sequence
  Synthetic Peptide
  MISC_FEATURE (3)..(3): Xaa is L-2,4-Diaminobutyric acid
  MISC_FEATURE (7)..(7): Xaa is L-2,4-Diaminobutyric acid
  MISC_FEATURE (14)..(15): Xaa is L-2,4-Diaminobutyric acid
  MISC_FEATURE (22)..(22): Xaa is L-2,4-Diaminobutyric acid
  MISC_FEATURE (26)..(26): Xaa is L-2,4-Diaminobutyric acid; all other amino acids area D-amino acids
KLXSLLXTLSAAKXXKLATLLXALSX

SEQ ID NO: 34 - protein, 26 aa, Artificial Sequence
  Synthetic Peptide
  MISC_FEATURE (3)..(3): Xaa is L-2,4-Diaminobutyric acid
  MISC_FEATURE (7)..(7): Xaa is L-2,4-Diaminobutyric acid
  MISC_FEATURE (13)..(16): Xaa is L-2,4-Diaminobutyric acid
  MISC_FEATURE (22)..(22): Xaa is L-2,4-Diaminobutyric acid
  MISC_FEATURE (26)..(26): Xaa is L-2,4-Diaminobutyric acid; all other amino acids are D-amino acids
KLXSLLXTLSAAXXXXLATLLXALSX

SEQ ID NO: 35 - protein, 26 aa, Artificial Sequence
  Synthetic Peptide
  MISC_FEATURE (3)..(3): Xaa is L-2,4-Diaminobutyric acid
  MISC_FEATURE (7)..(7): Xaa is L-2,4-Diaminobutyric acid
  MISC_FEATURE (13)..(13): Xaa is L-2,3-Diaminopropionic acid
  MISC_FEATURE (14)..(15): Xaa is L-2,4-Diaminobutyric acid
  MISC_FEATURE (16)..(16): Xaa is L-2,3-Diaminopropionic acid
  MISC_FEATURE (22)..(22): Xaa is L-2,4-Diaminobutyric acid
  MISC_FEATURE (26)..(26): Xaa is L-2,4-Diaminobutyric acid; all other amino acids are D-amino acid
KLXSLLXTLSAAXXXXLATLLXALSX

SEQ ID NO: 36 - protein, 26 aa, Artificial Sequence
  Synthetic Peptide
  MISC_FEATURE (3)..(3): Xaa is D-2,4-Diaminobutyric acid
  MISC_FEATURE (7)..(7): Xaa is D-2,4-Diaminobutyric acid
  MISC_FEATURE (13)..(16): Xaa is D-2,4-Diaminobutyric acid
  MISC_FEATURE (22)..(22): Xaa is D-2,4-Diaminobutyric acid
  MISC_FEATURE (26)..(26): Xaa is D-2,4-Diaminobutyric acid; all other amino acids are D-amino acids
KLXSLLXTLSAAXXXXLATLLXALSX

SEQ ID NO: 37 - protein, 26 aa, Artificial Sequence
  Synthetic Peptide
  MISC_FEATURE (3)..(3): Xaa is D-2,4-Diaminobutyric acid
  MISC_FEATURE (7)..(7): Xaa is D-2,4-Diaminobutyric acid
  MISC_FEATURE (13)..(13): Xaa is D-2,3-Diaminopropionic acid
  MISC_FEATURE (14)..(15): Xaa is D-2,4-Diaminobutyric acid
  MISC_FEATURE (16)..(16): Xaa is D-2,3-Diaminopropionic acid
  MISC_FEATURE (22)..(22): Xaa is D-2,4-Diaminobutyric acid
  MISC_FEATURE (26)..(26): Xaa is D-2,4-Diaminobutyric acid; all other amino acids are D-amino acids
KLXSLLXTLSAAXXXXLATLLXALSX

SEQ ID NO: 38 - protein, 26 aa, Artificial Sequence
  Synthetic Peptide
  MISC_FEATURE (1)..(26): All amino acids are D-amino acids
KLKSLLKTLSKAKAAKLKTLLKALSK

SEQ ID NO: 39 - protein, 26 aa, Artificial Sequence
  Synthetic Peptide
  MISC_FEATURE (1)..(26): All amino acids are D-amino acids
KLKSLLKTLSKAKAAKLKTLLKALSS

SEQ ID NO: 40 - protein, 26 aa, Artificial Sequence
  Synthetic Peptide
  MISC_FEATURE (1)..(26): All amino acids are D-amino acids
KLKSLLKTLSAAKKKKLATLLKALSK

SEQ ID NO: 41 - protein, 26 aa, Artificial Sequence
  Synthetic Peptide
  MISC_FEATURE (3)..(3): Xaa is L-2,4-Diaminobutyric acid
  MISC_FEATURE (7)..(7): Xaa is L-2,4-Diaminobutyric acid
  MISC_FEATURE (14)..(15): Xaa is L-2,4-Diaminobutyric acid
  MISC_FEATURE (22)..(22): Xaa is L-2,4-Diaminobutyric acid
  MISC_FEATURE (26)..(26): Xaa is L-2,4-Diaminobutyric acid; all other amino acids are D-amino acids
KLXSLLXTLSAAKXXKLATLLXALSX

SEQ ID NO: 42 - protein, 26 aa, Artificial Sequence
  Synthetic Peptide
  MISC_FEATURE (1)..(26): All amino acids are D-amino acids
KLKSLLKTLSAAKKKKLATLLKALSS

SEQ ID NO: 43 - protein, 26 aa, Artificial Sequence
  Synthetic Peptide
  MISC_FEATURE (1)..(26): All amino acids are D-amino acids
KLRSLLRTLSRAKAAKLRTLLRALSR

SEQ ID NO: 44 - protein, 26 aa, Artificial Sequence
  Synthetic Peptide
  MOD_RES (3)..(3): Xaa is D-Orn
  MOD_RES (7)..(7): Xaa is D-Orn
  MOD_RES (11)..(11): Xaa is D-Orn
  MOD_RES (18)..(18): Xaa is D-Orn
  MOD_RES (22)..(22): Xaa is D-Orn
  MOD_RES (26)..(26): Xaa is D-Orn; all other amino acids are D-amino acids
KLXSLLXTLSXAKAAKLXTLLXALSX

SEQ ID NO: 45 - protein, 26 aa, Artificial Sequence
  Synthetic Peptide
  MISC_FEATURE (3)..(3): Xaa is D-2,4-Diaminobutyric acid
  MISC_FEATURE (7)..(7): Xaa is D-2,4-Diaminobutyric acid
  MISC_FEATURE (14)..(15): Xaa is D-2,4-Diaminobutyric acid
  MISC_FEATURE (22)..(22): Xaa is D-2,4-Diaminobutyric acid
  MISC_FEATURE (26)..(26): Xaa is D-2,4-Diaminobutyric acid; all other amino acids are D-amino acids
KLXSLLXTLSAAAXXALATLLXALSX

SEQ ID NO: 46 - protein, 26 aa, Artificial Sequence
  Synthetic Peptide
  MOD_RES (3)..(3): Xaa is D-Orn
  MOD_RES (7)..(7): Xaa is D-Orn
  MOD_RES (11)..(11): Xaa is D-Orn
  MOD_RES (18)..(18): Xaa is D-Orn
  MOD_RES (22)..(22): Xaa is D-Orn
  MOD_RES (26)..(26): Xaa is D-Orn; all other amino acids are D-amino acids
KLXSLLXTLSXAAAAALXTLLXALSX

SEQ ID NO: 47 - protein, 26 aa, Artificial Sequence
  Synthetic Peptide
  MISC_FEATURE (3)..(3): Xaa is -2,4-Diaminobutyric acid
  MISC_FEATURE (7)..(7): Xaa is -2,4-Diaminobutyric acid
  MISC_FEATURE (11)..(11): Xaa is -2,4-Diaminobutyric acid
  MISC_FEATURE (18)..(18): Xaa is -2,4-Diaminobutyric acid
  MISC_FEATURE (22)..(22): Xaa is -2,4-Diaminobutyric acid
  MISC_FEATURE (26)..(26): Xaa is -2,4-Diaminobutyric acid; all other amino acids are D-amino acids
KLXSLLXTLSXAAAAALXTLLXALSX

SEQ ID NO: 48 - protein, 26 aa, Artificial Sequence
  Synthetic Peptide
  MISC_FEATURE (3)..(3): Xaa is -2,3-Diaminopropionic acid
  MISC_FEATURE (7)..(7): Xaa is -2,3-Diaminopropionic acid
  MISC_FEATURE (11)..(11): Xaa is -2,3-Diaminopropionic acid
  MISC_FEATURE (18)..(18): Xaa is -2,3-Diaminopropionic acid
  MISC_FEATURE (22)..(22): Xaa is -2,3-Diaminopropionic acid
  MISC_FEATURE (26)..(26): Xaa is -2,3-Diaminopropionic acid; all other amino acids are D-amino acids
KLXSLLXTLSXAAAAALXTLLXALSX

(19) United States
(12) Patent Application Publication
(10) Pub. No.: US 2022/0105150 A1
(43) Pub. Date: Apr. 7, 2022

(54) AMPHIPATHIC ALPHA-HELICAL ANTIMICROBIAL PEPTIDES TREAT INFECTIONS BY GRAM-NEGATIVE PATHOGENS

(71) Applicant: THE REGENTS OF THE UNIVERSITY OF COLORADO, a body corporate, Denver, CO (US)

(72) Inventors: Robert S. HODGES, Aurora, CO (US); Colin T. MANT, Denver, CO (US); Ziqing JIANG, Westminster, CO (US); Lajos GERA, Centennial, CO (US)

(21) Appl. No.: 17/423,611

(22) PCT Filed: Jan. 15, 2020
(86) PCT No.: PCT/US20/13628

§ 371 Date: Jul. 16, 2021

Related U.S. Application Data
(60) Provisional application No. 62/794,475, filed on Jan. 18, 2019.

(51) Int. Cl. A61K 38/16; A61P 31/04; A61K 47/60

(52) U.S. Cl. CPC A61K 38/16; A61K 47/60; A61P 31/04

(57) ABSTRACT

Antimicrobial agents, including antimicrobial peptides (AMPs), and uses thereof. Compositions and methods of using AMPs that demonstrate activity and improved therapeutic indices against microbial pathogens. The AMPs demonstrate the ability to not only maintain or improve antimicrobial activity against bacterial pathogens including Gram-negative microorganisms Acinetobacter-baumannii and Pseudomonas aeruginosa, but also significantly decrease hemolytic activity against human red blood cells. Specificity determinants within the AMPs change selectivity from broad spectrum antimicrobial activity to Gram-negative selectivity.

### CROSS-REFERENCE TO RELATED APPLICATIONS

This application claims the benefit of U.S. Provisional Patent Application Ser. No. 62/794,475, filed Jan. 18, 2019, the entire disclosure of which is incorporated herein by reference.

### STATEMENT OF FEDERALLY SPONSORED RESEARCH

This invention was made with government support under grant number R43 AI 131870 awarded by the National Institutes of Health. The U.S. government has certain rights in the invention.

### TECHNICAL FIELD

This disclosure relates to the field of antimicrobial peptides (AMPs) and treatments for microbial infections.

### BACKGROUND

The explosion of bacterial resistance to traditional antibiotics and a rapid increase in the incidence of multi-drug resistant microbes have created an urgency to develop new classes of antimicrobial agents (NO TIME TO WAIT: Securing the Future from Drug-Resistant Infections, Report to the Secretary-General Of The United Nations, April 2019; Antibiotic/Antimicrobial Resistance, CDC, 2019). There are now “Superbugs” resistant to most or all antibiotics (Coast, J., et al., Health Economics 1996, 5:217-26). The Infectious Diseases Society of America has reported that two-thirds of all health care associated infections are caused by six multi-drug resistant organisms referred to as “ESKAPE” pathogens consisting of two Gram-positive organisms, Enterococcus faecium and Staphylococcus aureus, and four Gram-negative organisms, Klebsiella pneumonia, Acinetobacter baumannii, Pseudomonas aeruginosa and Enterobacter species (sciencedaily.com/releases/2008/12/081201105706. htm). A recent study in Mexico demonstrated dramatic increases in the incidence of antibiotic-resistant species (Garza-Gonzalez, E., et al., Chemotherapy 2010, 56:275-79). Of 550 clinical isolates of A. baumannii and 250 clinical isolates of P. aeruginosa, 74% of A. baumannii, and 34% of P. aeruginosa were multi-drug resistant.

Polymyxin B and Polymyxin E (Colistin) are cationic peptides consisting of a cyclic heptapeptide with a tripeptide side chain acylated by a fatty acid chain at the amino terminus. These antibiotics were heavily used in the 1960s, but in the 1970s their clinical use was limited due to serious issues of nephrotoxicity and neurotoxicity (Biswas, S., et al., Expert Rev. Anti. Infect. Ther. 2012, 10:917-34; dx.doi.org/10.1155/2015/679109). The revival of these two peptides began in the mid-1990s, due to the lack of novel antibiotics effective against the increasingly-prevalent multi-drug resistant Gram-negative bacteria. Thus, these compounds have become antibiotics of last resort, needed for drug resistant bacteria but associated with a high incidence of toxicity. Resistance to these polymyxins could become a major global health challenge because virtually no new antibiotics are currently available for treating serious Gram-negative infections caused by polymyxin-resistant “superbugs.” Accordingly, there is a great need for additional therapeutic antimicrobial treatments effective against drug-resistant organisms.

### SUMMARY

Antimicrobial peptides (AMPs) are produced by bacteria, fungi, plants, insects, amphibians, crustaceans, fish and mammals, including humans, either constitutively or in response to the presence of a microbe (Jenssen, H., et al., Clin Microbiol Rev. 2006, 19:491-511). AMPs are rapidly bactericidal and generally have broad-spectrum activity. It is believed that the antimicrobial mechanism of action of cationic AMPs does not involve a stereoselective interaction with a chiral enzyme or lipid or protein since enantiomeric forms of AMPs with all-D-amino acids have shown equal activities compared to their all-L-enantiomers (Wade, D., et al, Proc. Natl. Acad. Sci. USA 1990, 87:4761-65; Cribbs, D. H., et al., J. Biol. Chem. 1997, 272:7431-36; Hong, S. Y., et al., Biochem. Pharmacol. 1999, 58:1775-80; Wakabayashi, H., et al., Antimicrob. Agents Chemother. 1999, 43:1267-69; De Lucca, A. J., et al., Med. Mycol. 2000, 38:301-8; Bland, J. M., et al., Mol. Cell. Biochem. 2001, 218:105-11; Hamamoto, K., et al., Microbiol. Immunol. 2002, 46:741-49, Elmquist, A., et al., Biol. Chem. 2003, 384:387-93; Chen, Y., et al., Chem. Biol. Drug Des. 2006, 67:162-73). Because their mode of action involves non-specific interactions with the cytoplasmic membrane of bacteria, bacteria rarely develop resistance to them. Additionally, all D-enantiomer peptides are resistant to proteolytic enzyme degradation, which enhances their potential use as therapeutic agents in mammals.

Unfortunately, native AMPs lack specificity between prokaryotic and eukaryotic cells, and are therefore too toxic to be used for systemic treatment of bacterial infections in mammals. This toxicity, which manifests as drug- and dose-limiting hemolysis of human red blood cells, has limited the development of a new class of antimicrobial agents based on these AMPs.

The present inventors have previously studied the number and location of positively-charged residues on the polar and non-polar face of AMPs, resulting in the development of new antimicrobial peptides with improvements in antimicrobial activity against Gram-negative pathogens and dramatic reductions in hemolytic activity and therefore unprecedented improvements in therapeutic indices.

This disclosure provides further refined AMPs that are highly effective and specific antimicrobial agents comprising peptides and peptide-containing compositions, and methods of inhibiting microorganisms, and treating a subject in need of antimicrobial therapy.

The antimicrobial peptides (AMPs) and compositions of this disclosure demonstrate activity and improved therapeutic indices against bacterial pathogens, particularly Gram-negative bacteria. These AMPs demonstrate the ability to not only maintain or improve antimicrobial activity against Gram-negative bacterial pathogens, but also significantly decrease the hemolysis of mammalian red blood cells. Thus, improved therapeutic indices are achieved by AMPs of this disclosure.

To overcome the significant mammalian toxicity of most of the known AMPs, the inventors developed the design concept of the “specificity determinant,” which refers to the substitution of positively charged amino acid residue(s) in the non-polar face of amphipathic alpha-helical or cyclic beta-sheet antimicrobial peptides to create selectivity between eukaryotic and prokaryotic membranes; that is, the antimicrobial activity of the AMPs of this disclosure is maintained, while the hemolytic activity or cell toxicity to mammalian cells is substantially decreased or eliminated.

This disclosure provides peptide antimicrobial agents and antimicrobial peptide compositions, as well as methods of inhibiting microorganisms and treating microbial infections, particularly infections by drug-resistant microorganisms. In an aspect of the claimed methods, a subject is treated by administering an AMP or a composition comprising an AMP of this disclosure. The antimicrobial peptides (AMPs) of this disclosure demonstrate activity and improved therapeutic indices against bacterial pathogens. These AMPs may demonstrate the ability to not only maintain or improve antimicrobial activity against bacterial pathogens, including Gram-negative microorganisms such as Acinetobacter baumannii and Pseudomonas aeruginosa, but also significantly decrease hemolysis of human red blood cells. Thus, the AMPs of this disclosure display significantly improved therapeutic indices.

Isolated antimicrobial peptides (AMPs) of this disclosure comprise 26 amino acid residues. These AMPs preferably include i) 2 specificity determinants; ii) non-naturally occurring, positively-charged amino acid residues; and, iii) a mixture of amino acid residues in the D- and L-enantiomeric form. In preferred forms, the non-naturally occurring, positively-charged amino acid residues, and the specificity determinants in these AMPs are selected from L-Diaminobutyric acid (L-Dab) and L-Diaminopropionic acid (L-Dap). In these AMPs the specificity determinants may be located at amino acid positions 13 and 16 of the AMP. In these AMPs, most of the amino acid residues are in the D-enantiomeric form, and at least 5 amino acid residues may be in the L-enantiomeric form. In these AMPs, most of the amino acid residues are in the D-enantiomeric form, and 5 or 6 amino acid residues may be in the L-enantiomeric form.

In these AMPs, most of the amino acid residues are in the D-enantiomeric form, and the amino acids residues located at positions 3, 7, 11, 18, 22, and 26, may be in the L-enantiomeric form. In these AMPs, most of the amino acid residues are in the D-enantiomeric form, and the amino acids residues located at positions 3, 7, 11, 14, 15, 22, and 26, may be in the L-enantiomeric form. Similarly, In these AMPs, most of the amino acid residues are in the D-enantiomeric form, and the amino acids residues located at positions 3, 7, 11, 18, 22, and 26, or positions 3, 7, 14, 15, 22, and 26, of the AMP may be L-Dab.

Isolated antimicrobial peptides (AMPs) of this disclosure comprise the amino acid sequence:

(SEQ ID NO: 1)
D-Lys-Xaa²-Xaa³-D-Ser-Xaa⁵-Xaa⁶-Xaa⁷-D-Thr-Xaa⁹-
D-Ser-Xaa¹¹-D-Ala-Xaa¹³-Xaa¹⁴-Xaa¹⁵-Xaa¹⁶-Xaa¹⁷-
Xaa¹⁸-D-Thr-Xaa²⁰-Xaa²¹-Xaa²²-D-Ala-Xaa²⁴-D-Ser-
Xaa²⁶

wherein: the ‘D-’ prefix denotes an amino acid residue in the D-enantiomeric form, and the prefix denotes an amino acid residue in the L-enantiomeric form; and,

Xaa², Xaa⁵, Xaa⁶, Xaa⁹, Xaa¹⁷, Xaa²⁰, Xaa²¹, and Xaa²⁴ are each independently selected from D-Leu (Leucine), D-Ile (Isoleucine), and D-Nle (Norleucine);

Xaa³, Xaa⁷, Xaa¹¹, Xaa¹⁸, and Xaa²² are each independently selected from L-Dab (Diaminobutyric acid), L-Dap (Diaminopropionic acid), D-Dab, D-Dap, D-Orn (Ornithine), D-Lys (Lysine), D-Ala (Alanine), and D-Arg (Arginine);

X¹³ and X¹⁶ are each independently selected from L-Dab, L-Dap, D-Dab, D-Dap, and D-Lys;

X¹⁴ and X¹⁵ are each independently selected from D-Lys, L-Dab, L-Dap, D-Dab, D-Dap, and D-Ala; and,

X²⁶ is selected from L-Dab, L-Dap, D-Dab, D-Dap, D-Cys (Cysteine), D-Ser (Serine), D-Orn, D-Lys, and D-Arg.

The peptides of this disclosure may include residues that disrupt the continuous hydrophobic surface that stabilizes the alpha-helical structure of AMPs that lack the “specificity determinants” (such as the naturally occurring peptides Piscidin 1 and/or Dermaseptin S4, and/or the all D-enantiomeric forms of these naturally occurring peptides). The peptides of this disclosure may include residues that reduce the hydrophobicity on the non-polar face and overall hydrophobicity of the peptide molecule (as measured by retention time at 25° C. by reversed-phase chromatography (RP-HPLC). The peptides of this disclosure may include residues that dramatically reduce peptide self-association in aqueous conditions (as measured by the temperature profiling in RP-HPLC procedure described in the Examples section of this disclosure). The peptides of this disclosure may have dramatically reduced toxicity to normal cells (as measured by hemolytic activity to human red blood cells at 37° C. after 18 hours). The peptides of this disclosure may have similar or substantially enhanced antimicrobial activity (compared to AMPs lacking specificity determinants, such as polymyxin B- and/or polymyxin E (Colistin)), and particularly with respect to bactericidal activity towards Gram-negative microbes. The peptides of this disclosure may have dramatically improved therapeutic indices (calculated by the ratio of hemolytic activity and antimicrobial activity (HC₅₀/MIC)) compared to AMPs lacking specificity determinants, such as polymyxin B- and/or Colistin. The peptides of this disclosure may have antimicrobial selectivity for Gram-negative pathogens resulting from similar or significantly decreased Gram-positive activity and hemolytic activity (compared to AMPs lacking specificity determinants, such as polymyxin B- and/or Colistin). The peptides of this disclosure may have antimicrobial activity against A. baumannii bacterial strains resistant to polymyxin B and/or Colistin antibiotics. The peptides of this disclosure may discriminate between eukaryotic and prokaryotic cell membranes. The peptides of this disclosure may have antimicrobial activity even in the presence of human serum.

Another aspect of this disclosure provides pharmaceutical compositions comprising at least one of the antimicrobial peptides of this disclosure, and a pharmaceutically acceptable carrier. These pharmaceutical compositions may include one or more AMPs having the amino acid sequence of any one of SEQ ID NOs:1-44.

Another aspect provides methods of preventing or treating an infection in a subject, including administering a therapeutically effective amount of a composition to the subject, wherein the composition comprises at least one antimicrobial peptide of this disclosure, and a pharmaceutically acceptable carrier. In these methods, the infecting microorganism may be Gram-negative bacteria. In these methods, the infecting microorganism may be an antibiotic-resistant microbe. The antibiotic resistant microbe may be a Gram-negative, antibiotic-resistant Acinetobacter baumannii or Pseudomonas aeruginosa pathogen. Alternatively or additionally, the antibiotic infecting microorganism may be a drug-resistant Gram-negative pathogen (such as a polymyxin B- and/or polymyxin E (Colistin)-resistant pathogen), or a polymyxin B- and/or polymyxin E-sensitive Gram-negative pathogen.

This disclosure also provides methods of inhibiting a microorganism, comprising contacting the microorganism with a composition comprising at least one AMP of this disclosure. In these methods, the AMP may be one or more of the peptides having the amino acid sequence of any one of SEQ ID NOS:1-44. In these methods, the AMP inhibits propagation of a prokaryote. The prokaryote may be a Gram-negative bacterium, which may include at least one of A. baumannii and P. aeruginosa bacterium.

One aspect of this disclosure provides an antimicrobial peptide (AMP) comprising an amino acid sequence having at least 85%, or at least 90%, or at least 95% sequence homology with a peptide selected from the group consisting of SEQ ID NOS:1-44, or functional analogues, derivatives, or fragments thereof, or pharmaceutically-acceptable salts thereof.

The AMPs of this disclosure may exhibit a therapeutic index (calculated by the ratio of hemolytic activity to antimicrobial activity (HC₅₀/MIC)) of at least 100. The AMPs of this disclosure may exhibit therapeutic index of between 100 and 1100. The AMPs of this disclosure may exhibit therapeutic index of between 700 and 1100, or between 950 and 1100.

The AMPs of this disclosure may exhibit at least a 20-fold increased selectivity for killing Gram-negative bacteria over Gram-positive bacteria.

The AMPs of this disclosure having the amino acid sequence of any one of SEQ ID NOs:1-42 may exhibit at least a 13-fold decrease in hemolysis of human red blood cells (measured as HC₅₀—the concentration of peptide that results in 50% hemolysis after 18 h at 37° C.) compared to hemolysis exhibited by SEQ ID NO:43.

Another aspect of this disclosure provides a pharmaceutical composition comprising at least one AMP of this disclosure and a pharmaceutically acceptable carrier. The pharmaceutical composition may be a mono-phasic pharmaceutical composition suitable for parenteral or oral administration consisting essentially of a therapeutically-effective amount of at least one AMP of this disclosure, and a pharmaceutically acceptable carrier. In these embodiments, the AMP may be one or more of the peptides having an amino acid sequence of any one of SEQ ID NOS:1-44.

Another aspect of this disclosure provides methods of preventing or treating a microbial infection comprising administering to a subject in need thereof a therapeutically effective amount of at least one AMP of this disclosure, or a pharmaceutical composition comprising the same. In these methods, the AMP administered may be one or more of the peptides having the sequence of SEQ ID NOS:1-44. In these methods, the microbial infection may be the result of an infecting bacteria, fungi, virus, or protozoa. The microbial infection may be a bacterial infection. The bacterial infection may be a Gram-negative bacterial infection. The bacterial infection may be an antibiotic resistant Gram-negative bacterial infection. The infecting microorganism may be at least one of Pseudomonas aeruginosa, and Acinetobacter baumannii. The infecting microorganism may be an antibiotic- or multi drug-resistant Pseudomonas aeruginosa, or Acinetobacter baumannii bacteria.

In these methods, the administration of the peptide or pharmaceutical composition may be made by an administration route selected from oral, topical, subcutaneous, intravenous, intraperitoneal, intramuscular, intradermal, intrasternal, intraarticular injection, and/or intrathecal. These peptides or pharmaceutical compositions may be administered in conjunction with one or more additional antimicrobial agents.

This disclosure also provides methods of preventing a microbial infection, or reducing the incidence of microbial infection, or slowing the growth of a microbial infection, in an individual comprising, or at risk of developing an infection, comprising administering an effective amount of at least one AMP of this disclosure, or a pharmaceutical composition comprising the same, to the individual in need thereof. The individual may be a surgical patient. The individual may be a hospitalized patient.

This disclosure also provides methods of combating a bacterial infection in a patient comprising applying at least one AMP of this disclosure, or a pharmaceutical composition comprising the same, to a body surface of the patient. The body surface may be a wound. The composition may be applied following an operation or surgery.

This disclosure also provides at least one AMP of this disclosure, or a pharmaceutical composition comprising the same, for use in the treatment of a microbial infection. This disclosure also provides the use of at least one peptide of this disclosure, or a pharmaceutical composition comprising the same, in the manufacture of a medicament for the prevention or treatment of a microbial infection.

This Summary is neither intended nor should it be construed as representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present invention is set forth in various levels of detail in this Summary as well as in the attached drawings and the Detailed Description and no limitation as to the scope of the present disclosure is intended by either the inclusion or non-inclusion of elements, components, etc. in this Summary. Additional aspects of the present invention will become more readily apparent from the Detailed Description, particularly when taken together with the drawings.

### BRIEF DESCRIPTION OF FIGURES

FIG. 1 is a helical wheel (upper panel) and helical net (lower panels) representation of amphipathic helical AMPs of this disclosure. In the helical wheel, the non-polar face is indicated as an arc (the Lys “specificity determinants” are at positions 13 and 16). Lys 1 is also on the non-polar face. The polar face is indicated as a black arc (positively charged residues at positions 3, 7, 11, 18, 22, and 26 are denoted by X). In the helical net (left side) the residues on the polar face are boxed and shown along the center of the net with the positively charged residues at positions 3, 7, 11, 18, 22 and 26. In the helical net (right side), the residues on the non-polar face are circled and shown along the center of the net. The Lys “specificity determinants” are at positions 13 and 16 in the center of the non-polar face between the hydrophobic clusters of Leu residues.

FIG. 2 is helical wheel (upper panels) and helical nets (lower panels) representations of an AMP with and without “specificity determinants.” Lys 13 and Lys 16 are in the center of the non-polar face (left side) between the two clusters of hydrophobic Leu residues. The right side show the helical net in the absence of specificity determinants, where Lys residues are replaced with Ala 13 and Ala 16, thus maintaining a continuous hydrophobic surface along the center of the helix. The positively charged residues on the polar face are indicated in the helical wheels with an X at positions 3, 7, 11, 18, 22, and 26.

FIGS. 3A and 3B show the percent lysis of human red blood cells versus peptide concentration of AMPs. FIG. 3A shows the percent lysis of sequences of the five peptides (all containing Lys specificity determinants at positions 13 and 16 of the non-polar face; see FIG. 1) shown in Table 1G. Peptide denotions in FIG. 3 have been abbreviated from those shown in Table 1G; for example, D87(Lys1-6 Arg-1) has been shortened to D87(6Arg-1), where 6Arg-1 denotes six Arg residues on the polar face at positions 3, 7, 11, 18, 22 and 26. FIG. 3B shows a comparison of percent lysis of the Dap- and Dab-containing peptides in the presence and absence of Lys specificity determinants at positions 13 and 16. The sequences of the peptides in FIG. 3B are shown in Table 1G. HC50 values (concentration of peptide that results in 50% hemolysis of human red blood cells after 18 h at 37° C.) derived from such data are shown in Table 4. Peptides without specificity determinants (A13/A16) are extremely hemolytic whereas peptides with specificity determinants (Lys13/Lys16) show minimal hemolytic activity (FIG. 13B).

FIG. 4 shows the relative hydrophobicity of AMPs as expressed by RP-HPLC elution time. Column: Zorbax 300 SB-C8, 150×2.1 mm ID, 5-μm particle size, 300-Å pore size; conditions, linear AB gradient (0.25% acetonitrile/min) at a flow-rate of 0.3 ml/min, where eluent A was 20 mM aq. TFA and eluent B was 20 mM TFA in acetonitrile and the temperature was 25° C. The sequences of the ten peptides (with seven peptides containing Lys specificity determinants at positions 13 and 16 of the non-polar face (see FIG. 1) are shown in Table 1G. Peptide denotions in FIG. 4 have been abbreviated from those shown in Table 1G; for example, D87(Lys1-6 Arg-1) has been shortened to D87(6 Arg-1). For the peptides without specificity determinants, we have added A13/A16 to the peptide denotions. It is interesting that the Dab containing peptides are more hydrophilic than the Dap containing peptides even thought the Dab containing peptides have an additional carbon atom per Dab residue. This shows that the Dab residues are stabilizing the polar face of the α-helix more than the Dap residues.

FIG. 5 shows the self-association of AMPs determined by temperature profiling in RP-HPLC. Retention behavior from eight AMPs after normalization to their retention times at 5° C. over the temperature range 5° C. to 41° C. in 2° C. increments or 5° C. to 75° C. in 10° C. increments (methodology details provided in Examples). The sequences of the eight peptides are shown in Table 1G. D85(A13/A16-6Orn-1), D86(A13/A16-6Dab-1), and D105(A13/A16-6Dap-1) do not contain Lys specificity determinants, whereas the remaining AMPs contain Lys specificity determinants at positions 13 and 16 on the non-polar face (see FIG. 1). Peptide denotions in FIG. 5 have been abbreviated from those shown in Table 1G; for example, D87(Lys1-6 Arg-1) has been shortened to D87(6 Arg-1). RC is a random coil control peptide used for RP-HPLC temperature profiling. The peptide self-association parameter, PA, represents the maximum change in peptide retention time relative to the random coil peptide, RC (PA values shown in Table 6). The inventors have published these results as Mant, C. T., et al. J. Med. Chem. 2019, 62:3354-66.

FIG. 6A shows helical wheels (upper panels) and helical nets (lower panels) representations of helical AMPs of this disclosure. In the helical wheels, the non-polar face is indicated as an arc (the specificity determinants are at positions 13 and 16). The polar face is indicated as a black arc (positively charged residues are denoted by X). In the helical nets, the positively charged residues on the polar face are boxed and other polar face residues are indicated with an open black box. The open boxes denote Lys residues on the non-polar face (Lys 1 and specificity determinants Lys 13 and Lys 16). The positions denoted by X are the positions of positively charged residues on the polar face at positions 3, 7, 11, 18, 22, and 26 (left wheel and left helical net) or at positions 3, 7, 14, 15, 22 and 26 (right helical wheel and right helical net). The potential i to i +3 or i to i +4 electrostatic repulsions between positively charged residues are shown as black dotted lines. FIG. 6B shows helical wheels (upper panels) and helical nets (lower panels) representations of helical AMPs of this disclosure. In the helical wheels, the non-polar face is indicated as an arc (the specificity determinants are at positions 13 and 16). The polar face is indicated as a black arc. In the helical nets, the residues on the non-polar face are circled with the Lys residues (Lys 1, and the specificity determinants Lys 13 and Lys 16) and the Leu residues in two clusters (L2, L5, L6, L9 for the N-terminal cluster and L17, L20, L21 and L24 for the C-terminal cluster). The black open boxes denote the positively charged residues on the polar face at positions 3, 7, 11, 18, 22, and 26 (left helical net) and positions 3, 7, 14, 15, 22 and 26 (right helical net). The potential i to i +3 or i to i +4 hydrophobic interactions between large hydrophobes are shown as black solid lines. FIG. 6C shows the percent lysis of human red blood cells versus peptide concentration of AMPs. The percent lysis of amphipathic α-helical antimicrobial peptides of this disclosure containing Dab and Dap residues on the polar face (sequences shown in Table 7). HC₅₀ values (concentration of peptide that results in 50% hemolysis of human red blood cells after 18 h at 37° C.) derived from such data are shown in Table 8. The inventors have published these results as Mant, C. T., et al. J. Med. Chem. And Drug Design 2019, Vol. 2, Issue 2 open access.

FIGS. 7A and 7B show the percent lysis of human red blood cells from four different blood donors (donors “A, B, C, and D”) by two antimicrobial peptides containing either 6-D-Dab or 6-L-Dab amino acid residues at positions 3, 7, 11,18, 22, and 26. The four panels of FIG. 7A show the differences in hemolytic activity between the two peptides in blood from the four different blood donors. FIG. 7B shows the differences in hemolytic activity for each peptide between the four blood donors.

### DETAILED DESCRIPTION

The terms and phrases used herein have their art-recognized meaning, which can be found by reference to standard texts, journal references, and contexts known to those skilled in the art.

As used herein, the singular forms “a”, “an”, and “the” include plural reference unless the context clearly dictates otherwise. For example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “containing” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the pertinent art.

Whenever a range of values is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

The following definitions are provided to clarify terms used in the context of this disclosure.

As used herein, the term “amino acid” is intended to refer to any natural or unnatural amino acid, whether made naturally or synthetically, including those in the L- or D-enantiomeric configurations. The term can also encompass amino acid analog compounds used in peptidomimetics or in peptoids. The term can include a modified or unusual amino acid or a synthetic derivative of an amino acid, e.g. diaminobutyric acid and diaminopropionic acid and the like. The antimicrobial peptides comprise amino acids linked together by peptide bonds. The peptides are in general in alpha helical conformation under hydrophobic conditions. Sequences are conventionally given from the amino terminus to the carboxyl terminus. Unless otherwise noted, the amino acids are D-amino acids. When all the amino acids are of L-configuration, the peptide is said to be an L-enantiomer. When all the amino acids are of D-configuration, the peptide is said to be a D-enantiomer.

The term “hemolytic concentration-50” or “HC₅₀” refers to the peptide concentration that causes 50% hemolysis of erythrocytes after 18 h. HC₅₀ was determined from a plot of percent lysis versus peptide concentration (μM). For comparison, the inventors also determined the hemolytic activity after 18 hours at 37° C. Hemolysis can be determined with red blood cells (RBC) from various species including human red blood cells (hRBC). Therapeutically effective AMPs of this disclosure are, in most instances, so non-hemolytic to human red blood cells that the HC₅₀ value could not be observed. Therefore, the HC₅₀ value was calculated by extrapolation.

The term “therapeutic index” (TI) is the ratio of HC₅₀ over the geometric mean of the minimal inhibitory concentration (MIC_{GM}) of an antimicrobial agent. Larger values generally indicate greater antimicrobial specificity.

The term “stability” can refer to an ability to resist degradation, to persist in a given environment, and/or to maintain a particular structure. For example, a peptide property of stability can indicate resistance to proteolytic degradation and to maintain an alpha-helical structural conformation.

The following abbreviations are useful: A, Ala, Alanine; M, Met, Methionine; C, Cys, Cysteine; D, Asp, Aspartic Acid; E, Glu, Glutamic Acid; F, Phe, Phenylalanine; G, Gly, Glycine; H, His, Histidine; I, Ile, Isoleucine; K, Lys, Lysine; L, Leu, Leucine; N, Asn, Asparagine; Nle, Norleucine; O, Orn, Ornithine; P, Pro, Proline; Q, Gln, Glutamine; R, Arg, Arginine; S, Ser, Serine; T, Thr, Threonine; V, Val, Valine; W, Trp, Tryptophan; Y, Tyr, Tyrosine; Dab, 2,4-Diaminobutyric acid (formerly abbreviated “Dbu” in older scientific and patent literature); Dap, 2,3-Diaminopropionic acid (formerly abbreviated “Dpr” in older scientific and patent literature); RP-HPLC, reversed-phase high performance liquid chromatography; MIC, minimal inhibitory concentration; HC₃₀ hemolytic concentration-30; HC₅₀ hemolytic concentration-50; CD, circular dichroism spectroscopy; TFE, 2,2,2-trifluoroethanol; TFA, trifluoroacetic acid; RBC, red blood cells; hRBC, human red blood cells.

The term “antimicrobial activity” refers to the ability of a peptide to modify a function or metabolic process of a target microorganism, for example to at least partially affect replication, vegetative growth, toxin production, survival, viability in a quiescent state, or other attribute. The term relates to inhibition of growth of a microorganism. In aspects of the claimed peptides and methods, antimicrobial activity relates to the ability of a peptide to kill at least one bacterial species. The bacterial species may be a Gram-negative bacteria. The term can be manifested as microbicidal or microbistatic inhibition of microbial growth.

The phrase “improved biological property” is meant to indicate that a test peptide exhibits less hemolytic activity and/or better antimicrobial activity, or better antimicrobial activity and/or less hemolytic activity, compared to a control peptide (for example, Colistin or polymyxin B), when tested by the protocols described herein or by any other art-known standard protocols. In general, the improved biological property of the peptide is reflected in the therapeutic index (TI) value which is better than that of the control peptide.

The term “microorganism” herein refers broadly to bacteria, fungi, viruses, and protozoa. In particular, the term is applicable for a microorganism having a cellular or structural component of a lipid bilayer membrane. The membrane may be a cytoplasmic membrane. Pathogenic bacteria, fungi, viruses, and protozoa as known in the art are generally encompassed. Bacteria can include Gram-negative and Gram-positive bacteria in addition to organisms classified in orders of the class Mollicutes and the like, such as species of the Mycoplasma and Acholeplasma genera. Specific examples of Gram-negative bacteria include, but are not limited to, Escherichia coli, Pseudomonas aeruginosa, Acinetobacter baumannii, Salmonella spp., Haemophilus influenzae, Neisseria spp., Vibrio cholerae, Vibrio parahaemolyticus and Helicobacter pylori. Examples of Gram-positive bacteria include, but are not limited to, Staphylococcus aureus, Staphylococcus epidermidis, Streptococcus agalactiae, Group A Streptococcus, Streptococcus pyogenes, Enterococcus faecalis, Group B Gram-positive Streptococcus, Corynebacterium xerosis, and Listeria monocytogenes. Examples of fungi include yeasts such as Candida albicans. Examples of viruses include measles virus, herpes simplex virus (HSV-1 and -2), herpes family members (HIV, hepatitis C, vesicular stomatitis virus (VSV), visna virus, and cytomegalovirus (CMV). Examples of protozoa include Giardia.

“Therapeutically effective amount” as used herein, refers to an amount of formulation, composition, or reagent in a pharmaceutically acceptable carrier or a physiologically acceptable salt of an active compound that is of sufficient quantity to ameliorate the undesirable state of the subject, patient, animal, material, or object so treated. “Ameliorate” refers to a lessening of the detrimental effect of the disease state or disorder, or reduction in contamination, in the recipient of the treatment.

“Pharmaceutical agent or drug” as used herein, refers to a chemical compound or composition capable of inducing a desired therapeutic or prophylactic effect when properly administered to a subject.

“Pharmaceutically acceptable carrier” as used herein, refers to conventional pharmaceutical carriers useful in the methods disclosed herein. Remington's Pharmaceutical Sciences, by E. W. Martin, Mack Publishing Co., Easton, Pa., 15th Edition (1975), describes compositions and formulations suitable for pharmaceutical delivery of TCR peptides and additional pharmaceutical agents. In general, the nature of the carrier will depend on the particular mode of administration employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain non-toxic auxiliary substances, such as wetting or emulsifying agents, preservatives, salts, amino acids, and pH buffering agents and the like, for example sodium or potassium chloride or phosphate, Tween, sodium acetate or sorbitan monolaurate.

Antimicrobial peptides (AMPs) of this disclosure have antimicrobial activity by themselves, or when covalently conjugated or otherwise coupled or associated with another molecule, e.g., polyethylene glycol or a carrier protein such as bovine serum albumin, so long as the peptides are positioned such that they can come into contact with a cell or unit of the target microorganism. These peptides may be modified by methods known in the art provided that the antimicrobial activity is not destroyed or substantially compromised. Thus, also contemplated within the context of the inventive AMPs, methods, and compositions of this disclosure is the modification of any antimicrobial peptide described herein, by chemical or genetic means. Examples of such modification include construction of peptides of partial or complete sequence with non-natural amino acids and/or natural amino acids in L- or D-enantiomeric forms. Furthermore, the polypeptides may be modified to contain carbohydrate or lipid moieties, such as sugars or fatty acids, covalently linked to the side chains or the N- or C-termini of the amino acids. In addition, the polypeptides may be modified by glycosylation and/or phosphorylation. In addition, the polypeptides may be modified to enhance solubility and/or half-life upon being administered. For example, polyethylene glycol (PEG) and related polymers have been used to enhance solubility and the half-life of protein therapeutics in the blood. Accordingly, the antimicrobial peptides of this disclosure may be modified by PEG polymers and the like. “PEG” or “PEG polymers” means a residue containing poly(ethylene glycol) as an essential part. Such a PEG can contain further chemical groups which are necessary for the therapeutic activity of the peptides of this disclosure; which results from the chemical synthesis of the molecule; or which is a spacer for optimal distance of the parts of the molecule from one another. In addition, such a PEG can consist of one or more PEG side-chains which are linked together. PEG groups with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEGs usually have 2 to 8 arms and are described in, for example, U.S. Pat. No. 5,932,462 which is incorporated herein for this purpose. Especially preferred are PEGs with two, three, or four PEG side-chains (PEG2, PEG3, PEG4, respectively) linked via the primary amino groups of a lysine (Monfardini, C, et al., Bioconjugate Chem. 6 (1995) 62-69). The term “PEG” is used broadly to encompass any polyethylene glycol molecule, wherein the number of ethylene glycol (EG) units is at least 460, preferably 460 to 2300 and especially preferably 460 to 1840 (230 EG units refers to a molecular weight of about 10 kDa). The upper number of EG units is only limited by solubility of the PEGylated peptides of this disclosure. Usually PEGs which are larger than PEGs containing 2300 units are not used. Preferably, a PEG used in the invention terminates on one end with hydroxy or methoxy (methoxy PEG, mPEG) and is on the other end covalently attached to a linker moiety via an ether oxygen bond. The polymer is either linear or branched. Branched PEGs are e.g. described in Veronese, F. M., et al., Journal of Bioactive and Compatible Polymers 12 (1997) 196-207. Suitable processes and preferred reagents for the production of PEGylated peptides and variants of this disclosure are described in US Patent Pub. No. 2006/0154865. It is understood that modifications, for example, based on the methods described by Veronese, F. M., Biomaterials 22 (2001) 405-417, can be made in the procedures so long as the process results in PEGylated peptides of this disclosure. Particularly preferred processes for the preparation of PEGylated peptides of this disclosure are described in US Patent Publication No. 2008/0119409, which is incorporated herein by reference for this purpose.

Additionally or alternatively, the antimicrobial peptides of this disclosure may be fused to one or more domains of an Fc region of human IgG proteins. Antibodies comprise two functionally independent parts, a variable domain (known as “Fab”) that binds an antigen, and a constant domain (known as “Fc”) that is involved in effector functions such as complement activation and attack by phagocytic cells. An Fc has a long serum half-life, whereas a Fab is short-lived (Capon et al., 1989, Nature 337:525-31). When constructed together with an antimicrobial protein of this disclosure, an Fc domain can provide longer half-life or incorporate such functions as Fc receptor binding, protein A binding, complement fixation, and perhaps even blood-brain barrier, or placental transfer. In one example, a human IgG hinge, CH2, and CH3 region may be fused at either the amino-terminus or carboxyl-terminus of the peptides of this disclosure using methods known to the skilled artisan. The resulting fusion polypeptide may be purified by use of a Protein A affinity column. Peptides and proteins fused to an Fc region have been found to exhibit a substantially greater half-life in vivo than the unfused counterpart. Also, a fusion to an Fc region allows for dimerization/multimerization of the fusion polypeptide. The Fc region may be a naturally occurring Fc region, or may be altered to improve certain qualities, such as therapeutic qualities, circulation time, or reduced aggregation.

The polypeptides of this disclosure may also be modified to contain sulfur, phosphorous, halogens, metals, etc. Amino acid mimics may be used to produce polypeptides, and therefore, the polypeptides of this disclosure may include amino acid mimics that have enhanced properties, such as resistance to degradation.

The peptides of this disclosure may be isolated or purified. These peptides may be synthetic and can be produced by peptide synthesis techniques or by recombinant expression technology as understood in the art. As used herein, the term “purified” can be understood to refer to a state of enrichment or selective enrichment of a particular component relative to an earlier state of crudeness or constituency of another component. This term can be considered to correspond to a material that is at least partially purified as opposed to a state of absolute purity. For example, a peptide composition may be considered purified even if the composition does not reach a level of one hundred percent purity with respect to other components in the composition.

As used herein, the term “specificity determinant(s)” refers to positively charged amino acid residue(s) (including, for example, lysine, arginine, ornithine, diaminopropionic acid, or diaminobutyric acid) in the non-polar face of AMPs that could decrease hemolytic activity/toxicity but increase or maintain the same level of antimicrobial activity, thus increasing the therapeutic index of the AMP.

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

When a group of substituents is disclosed herein, it is understood that all individual members of that group and all subgroups, including any isomers, enantiomers, and diastereomers of the group members, are disclosed separately.

When a Markush group or other grouping is used herein, all individual members of the group and all combinations and subcombinations possible of the group are intended to be individually included in this disclosure. When a compound is described herein such that a particular isomer, enantiomer, or diastereomer of the compound is not specified, for example, in a formula or in a chemical name, that description is intended to include each isomer and enantiomer of the compound described individually, or in any combination. Additionally, unless otherwise specified, all isotopic variants of compounds disclosed herein are intended to be encompassed by the disclosure. As a brief illustration, it will be understood that any one or more hydrogens in a molecule disclosed can be replaced with deuterium or tritium.

Isotopic variants of a molecule are generally useful as standards in assays for the molecule and in chemical and biological research related to the molecule or its use. Methods for making such isotopic variants are known in the art.

One of ordinary skill in the art will appreciate that starting materials, biological and chemical materials, biological and chemical reagents, synthetic methods, purification methods, analytical methods, assay methods, and biological methods other than those specifically exemplified can be employed in the practice of the invention without resort to undue experimentation. All art-known functional equivalents of such materials and methods are included in this disclosure.

The terms and expressions employed herein are used as terms of description and not of limitation, and there is no intention in the use of such terms and expressions of excluding any equivalents of the features shown and described or portions thereof, but it is recognized that various modifications are possible within the scope of the invention claimed. Thus, although the invention has been disclosed by various aspects that may include preferred embodiments and aspects, modifications and variations of the concepts herein disclosed may be resorted to by those skilled in the art. Such modifications and variations are within the scope of this invention as defined by the appended claims.

Antimicrobial Peptides (AMPs) of this Disclosure

The antimicrobial peptides of this disclosure comprise 26-mer peptides comprising the amino acid sequence:

(SEQ ID NO: 1)
D-Lys-Xaa²-Xaa³-D-Ser-Xaa⁵-Xaa⁶-Xaa⁷-D-Thr-Xaa⁹-
D-Ser-Xaa¹¹-D-Ala-Xaa¹³-Xaa¹⁴-Xaa¹⁵-Xaa¹⁶-Xaa¹⁷-
Xaa¹⁸-D-Thr-Xaa²⁰-Xaa²¹-Xaa²²-D-Ala-Xaa²⁴-D-Ser-
Xaa²⁶

Wherein: the ‘D-’ prefix denotes an amino acid residue in the D-enantiomeric form and the ‘L-’ prefix denotes an amino acid residue in the L-enantiomeric form; and

Xaa², Xaa⁵, Xaa⁶, Xaa⁹, Xaa¹⁷, Xaa²⁰, Xaa²¹, and Xaa²⁴ are each independently selected from D-Leu (Leucine), D-Ile (Isoleucine), and D-Nle (Norleucine);

Xaa³, Xaa⁷, Xaa¹¹, Xaa¹⁸, and Xaa²² are each independently selected from L-Dab (Diaminobutyric acid), L-Dap (Diaminopropionic acid), D-Dab, D-Dap, D-Orn (Ornithine), D-Lys (Lysine), D-Ala (Alanine), and D-Arg (Arginine);

X¹³ and X¹⁶ are each independently selected from L-Dab, L-Dap, D-Dab, D-Dap, and D-Lys;

X¹⁴ and X¹⁵ are each independently selected from D-Lys, L-Dab, L-Dap, D-Dab, D-Dap, and D-Ala; and,

X²⁶ is selected from L-Dab, L-Dap, D-Dab, D-Dap, D-Cys (Cysteine), D-Ser (Serine), D-Orn, D-Lys, and D-Arg.

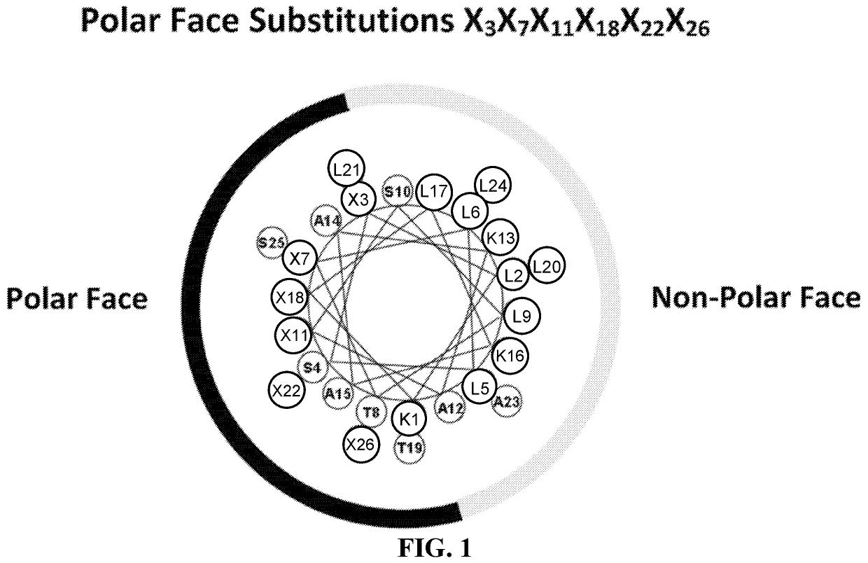

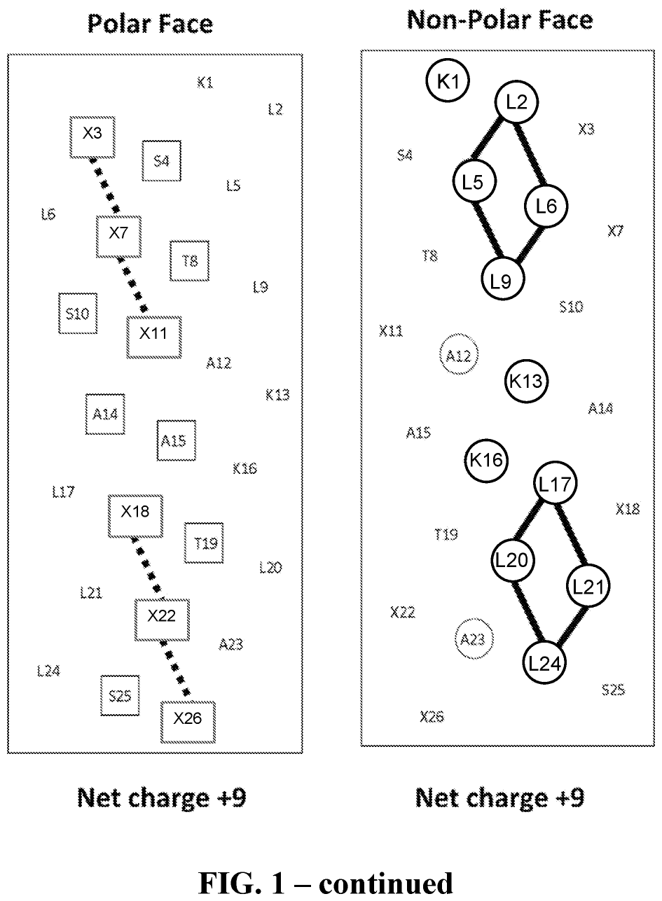

A series of peptides (shown in Table 1A) were designed and tested to show the effects of pegylation of AMPs of this disclosure substitutions to the specificity determinants at positions 13 and 16 of these 26-mer AMPs. In Table 1A, the ‘D-’ denotes that all amino acid residues in each peptide are in the D-enantiomeric conformation except for the L-Dab and L-Dap residues which are in the L-enantiomeric conformation. Specificity determinants are positively charged residues in the center of the non-polar face of the 26-mer AMPs (i.e., Lys13 and Lys16). Peptide sequences are shown using the one-letter code for all amino acid residues except where the three-letter code is used. “Ac” denotes N^{α}-acetyl and amide denotes C^{α}-amide. Amino acid positions 1, 3, 7, 11, 18, 22, and 26 are positively-charged residues (L-Dab and L-Dap) on the polar face of the amphipathic α-helix (see FIG. 1); −1 denotes 6 positively-charged residues on the polar face at positions 3, 7, 11, 18, 22, and 26, or 5 positively-charged residues on the polar face at positions 3, 7, 11, 18, and 22 (position 26 is substituted by Cys). PEG1 and PEG2 are attached to the alpha-amino group at position 1. PEG3 and PEG4 are attached to the SH group of Cysteine at position 26.

Another series of peptides (shown in Table 1B) were designed and tested to show the effects of the type of hydrophobic amino acids present on the non-polar face of the AMPs on antimicrobial and hemolytic activity. In Table 1B, the ‘D-’ denotes that all amino acid residues in each peptide are in the D-enantiomeric conformation except for L-Dab and L-Dap residues, which are in the L-enantiomeric conformation. Specificity determinants are positively charged residues in the center of the non-polar face of the 26-mer AMPs (i.e., Lys13 and Lys16). Peptide sequences are shown using the one-letter code for all amino acid residues except for L-Dap, L-Dab, leucine (Leu) residues, isoleucine (Ile) and norleucine residues (Nle) on the non-polar face at positions 2, 5, 6, 9, 17, 20, 21, and 24 where the three-letter code is used. “Ac” denotes N^{α}-acetyl and amide denotes C^{α}-amide. Positions 3, 7, 11, 18, and 22 are positively-charged residues (L-Dab and L-Dap) on the polar face of the amphipathic α-helix (see FIG. 1); '1 denotes 5 positively charged residues on the polar face at positions 3, 7, 11, 18 and 22. −2 denotes 5 positively charged residues on the polar face at positions 3, 7, 14, 15 and 22.

Another series of peptides (shown in Table 1C) were designed and tested to show the effects of L- and D-diaminobutyric and diaminopropionic acid residues on the polar face of AMPs on hemolytic activity and antimicrobial activity to treat the Gram-negative pathogen, Acinetobacter baumannii. In Table 1C, the ‘D-’ denotes that all amino acid residues in each peptide are in the D-enantiomeric conformation except for L-Dab and L-Dap residues, which are in the L-enantiomeric conformation. Specificity determinants are positively charged residues in the center of the non-polar face of the 26-mer AMPs (i.e., Lys13 and Lys16). Peptide sequences are shown using the one-letter code for all amino acid residues except for L-Dap, L-Dab, and serine (Ser) residues where the three-letter code is used. “Ac” denotes N^{α}-acetyl and amide denotes C^{α}-amide. Positions 3, 7, 11, 14, 15, 18, 22, or 26 are positively-charged residues (L-Dab and L-Dap) on the polar face of the amphipathic α-helix (see FIG. 1); −1 denotes 6 positively-charged residues on the polar face at positions 3, 7, 11, 18, 22 and 26 or 5 positively-charged residues on the polar face at positions 3, 7, 11, 18 and 22 (position 26 is substituted by Ser). −2 denotes 6 positively charged residues on the polar face at positions 3, 7, 14, 15, 22 and 26 or 5 positively-charged residues on the polar face at positions 3, 7, 14, 15, 22 (position 26 substituted by Ser).

TABLE 1A
Pegylation of peptides D86, D102, and D131 (SEQ ID NOs: 2, 3, 4).

| Laboratory Peptide | Sequence | SEQ |
|---|---|---|
| Name | With specificity determinants (Lys13/Lys16) | ID |
| Amino acid positions | 1 3 7 11 18 22 26 | NO |
| D86(Lys1- Dab-1) | Ac-KL(L-Dab)SLL(L-Dab)TLS(L-Dab)AKAAKL(L-Dab)TLL(L-Dab)ALS(L-Dab)-amide | 2 |
| D86(PEG1)(Lys¹-6Dab- | (PEG1)-KL(L-Dab)SLL(L-Dab)TLS(L-Dab)AKAAKL(L-Dab)TLL(L-Dab)ALS(L-Dab)-amide | 2 |
| 1) | | |
| D86(PEG2)(Lys¹-6Dab- | (PEG2)-KL(L-Dab)SLL(L-Dab)TLS(L-Dab)AKAAKL(L-Dab)TLL(L-Dab)ALS(L-Dab)-amide | 2 |
| 1) | | |
| D102(Lys¹Ser26Cys) | Ac-KL(L-Dab)SLL(L-Dab)TLS(L-Dab)AKAAKL(L-Dab)TLL(L-Dab)ALS(Cys)-amide | 3 |
| (5Dab-1) | | |
| D102(Lys¹Ser26Cys- | Ac-KL(L-Dab)SLL(L-Dab)TLS(L-Dab)AKAAKL(L-Dab)TLL(L-Dab)ALS(Cys-PEG3)-amide | 3 |
| PEG3)(5Dab-1) | | |
| D102(Lys¹Ser26Cys- | Ac-KL(L-Dab)SLL(L-Dab)TLS(L-Dab)AKAAKL(L-Dab)TLL(L-Dab)ALS(Cys-PEG4)-amide | 3 |
| PEG4)(5Dab-1) | | |
| D131(Lys¹Ser26Cys) | Ac-KL(L-Dap)SLL(L-Dap)TLS(L-Dap)AKAAKL(L-Dap)TLL(L-Dap)ALS(Cys)-amide | 4 |
| (5Dap-1) | | |
| D131(Lys¹Ser26Cys- | Ac-KL(L-Dap)SLL(L-Dap)TLS(L-Dap)AKAAKL(L-Dap)TLL(L-Dap)ALS(Cys-PEG3)-amide | 4 |
| PEG3)(5Dap-1) | | |
| D131(Lys¹Ser26Cys- | Ac-KL(L-Dap)SLL(L-Dap)TLS(L-Dap)AKAAKL(L-Dap)TLL(L-Dap)ALS(Cys-PEG4)-amide | 4 |
| PEG4)(5Dap-1) | | |

TABLE 1B
Polar face substitutions of positively charged residues Dab and Dap with a non-polar face consisting of Ile, Leu, and
Nle residues.

| Laboratory | | |
|---|---|---|
| Peptide | Sequence | SEQ ID |
| Name | With specificity determinants (Lys13/Lys16) | NO |
| Amino | | |
| acid | | |
| positions | 1 3 7 11 18 22 26 | |
| D132(Lys¹Lys¹³Lys¹⁶Ser²⁶- | Ac-K(Nle)(L-Dab)S(Nle)(Nle)(L-Dab)T(Nle)S(L-Dab)AKAAK(Nle)(L-Dab)T(Nle)(Nle)(L-Dab)A(Nle)SS-amide | 5 |
| 8Nle-5Dab-1) | | |
| D133(Lys¹ys¹³Lys¹⁶Ser²⁶- | Ac-K(Nle)(L-Dap)S(Nle)(Nle)(L-Dap)T(Nle)S(L-Dap)AKAAK(Nle)(L-Dap)T(Nle)(Nle)(L-Dap)A(Nle)SS-amide | 6 |
| 8Nle-5Dap-1) | | |
| D102(Lys¹Lys¹³Lys¹⁶Ser²⁶- | Ac-K(Leu)(L-Dab)S(Leu)(Leu)(L-Dab)T(Leu)S(L-Dab)AKAAK(Leu)(L-Dab)T(Leu)(Leu)(L-Dab)A(Leu)SS-amide | 7 |
| 8Leu-5Dab-1) | | |
| D136(Lys¹Lys¹³Lys¹⁶Ser²⁶- | Ac-K(Ile)(L-Dab)S(Ile)(Ile)(L-Dab)T(Ile)S(L-Dab)AKAAK(Ile)(L-Dab)T(Ile)(Ile)(L-Dab)A(Ile)SS-amide | 8 |
| 8Ile-5Dab-1) | | |
| Amino | | |
| acid | | |
| positions | 1 3 7 13 14 15 16 22 26 | |
| D134(Lys¹Lys¹³Lys¹⁶Ser²⁶- | Ac-K(Nle)(L-Dab)S(Nle)(Nle)(L-Dab)T(Nle)SAAK(L-Dab)(L-Dab)K(Nle)AT(Nle)(Nle)(L-Dab)A(Nle)SS-amide | 9 |
| 8Nle-5Dab-2) | | |
| D135(Lys¹Lys¹³Lys¹⁶Ser²⁶- | Ac-K(Nle)(L-Dap)S(Nle)(Nle)(L-Dap)T(Nle)SAAK(L-Dap)(L-Dap)K(Nle)AT(Nle)(Nle)(L-Dap)A(Nle)SS-amide | 10 |
| 8Nle-5Dap-2) | | |
| D104(Lys¹Lys¹³Lys¹⁶Ser²⁶- | Ac-K(Leu)(L-Dab)S(Leu)(Leu)(L-Dab)T(Leu)SAAK(L-Dab)(L-Dab)K(Leu)AT(Leu)(Leu)(L-Dab)A(Leu)SS-amide | 11 |
| 8Leu-5Dab-2) | | |
| D137(Lys¹Lys¹³Lys¹⁶Ser²⁶- | Ac-K(Ile)(L-Dab)S(Ile)(Ile)(L-Dab)T(Ile)SAAK(L-Dab)(L-Dab)K(Ile)AT(Ile)(Ile)(L-Dab)A(Ile)SS-amide | 12 |
| 8Ile-5Dab-2) | | |
| D250(Lys¹Lys¹³Lys^{l6}Ser²⁶- | Ac-K(Nle)(D-Dab)S(Nle)(Nle)(D-Dab)T(Nle)SAAK(D-Dab)(D-Dab)K(Nle)AT(Nle)(Nle)(D-Dab)A(Nle)SS-amide | 13 |
| 8Nle-5Dab-2) | | |
| D251(Lys¹Lys¹³Lys¹⁶Ser²⁶- | Ac-K(Nle)(D-Dap)S(Nle)(Nle)(D-Dap)T(Nle)SAAK(D-Dap)(D-Dap)K(Nle)AT(Nle)(Nle)(D-Dap)A(Nle)SS-amide | 14 |
| 8Nle-5Dap-2) | | |
| D252(Lys¹Lys¹³Lys¹⁶Ser²⁶- | Ac-K(Leu)(D-Dab)S(Leu)(Leu)(D-Dab)T(Leu)SAAK(D-Dab)(D-Dab)K(Leu)AT(Leu)(Leu)(D-Dab)A(Leu)SS-amide | 15 |
| 8Leu-5Dab-2) | | |
| D253(Lys¹Lys¹³Lys¹⁶Ser²⁶- | Ac-K(Ile)(D-Dab)S(Ile)(Ile)(D-Dab)T(Ile)SAAK(D-Dab)(D-Dab)K(Ile)AT(Ile)(Ile)(D-Dab)A(Ile)SS-amide | 16 |
| 8Ile-5Dab-2) | | |

TABLE 1C
Substitution of L- and D-diaminobutyric and diaminopropionic acid residues on the polar face of
amphipathic α-helical antimicrobial peptides.

| Laboratory | | | SEQ |
|---|---|---|---|
| Peptide | Peptide | | ID |
| Name | Mass | Sequence | NO |
| Amino | −I Dab | Lys residues at positions 1, 13 and 16 | |
| acid positions | Series | 1 3 7 11 18 22 26 | |
| D102(L-Dab^{3,7,11,18,22}) | 2684.3 | Ac-KL(L-Dab)SLL(L-Dab)TLS(L-Dab)AKAAKL(L-Dab)TLL(L-Dab)ALSS-amide | 7 |
| D200(D-Dab^{3,7,11,18,22}) | 2684.3 | Ac-KL(D-Dab)SLL(D-Dab)TLS(D-Dab)AKAAKL(D-Dab)TLL(D-Dab)ALSS-amide | 17 |
| D201(L-Dab^{3,22}) | 2684.3 | Ac-KL(L-Dab)SLL(D-Dab)TLS(D-Dab)AKAAKL(D-Dab)TLL(L-Dab)ALSS-amide | 18 |
| (D-Dab^{7,11,18}) | | | |
| D202(L-Dab^{7,11,18}) | 2684.3 | Ac-KL(D-Dab)SLL(L-Dab)TLS(L-Dab)AKAAKL(L-Dab)TLL(D-Dab)ALSS-amide | 19 |
| (D-Dab^{3,22}) | | | |
| Amino | −2 Dab | Lys residues at positions 1, 13, 16 | |
| acid positions | Series | 1 3 7 14 15 22 26 | |
| D104(L-Dab^{3,7,14,15,22}) | 2684.3 | Ac-KL(L-Dab)SLL(L-Dab)TLSAA K(L-Dab)(L-Dab) KLATLL(L-Dab)ALSS-amide | 11 |
| D210(D-Dab^{3,7,14,15,22}) | 2684.3 | Ac-KL(D-Dab)SLL(D-Dab)TLSAA K(D-Dab)(D-Dab) KLATLL(D-Dab)ALSS-amide | 20 |
| D211(L-Dab^{3,7,22}) | 2684.3 | Ac-KL(L-Dab)SLL(L-Dab)TLSAA K(D-Dab)(D-Dab) KLATLL(L-Dab)ALSS-amide | 21 |
| (D-Dab^{14,15}) | | | |
| D212(L-Dab^{14,15}) | 2684.3 | Ac-KL(D-Dab)SLL(D-Dab)TLSAA K(L-Dab)(L-Dab) KLATLL(D-Dab)ALSS-amide | 22 |
| (D-Dab^{3,7,22}) | | | |
| Amino | −1 Dap | Lys residues at positions 1, 13 and 16 | |
| acid positions | Series | 1 3 7 11 18 22 26 | |
| D105 | 2613.1 | Ac-KL(L-Dap)SLL(L-Dap)TLS(L-Dap)AKAAKL(L-Dap)TLL(L-Dap)ALS(L-Dap)- | 23 |
| (L-Dap^{3,7,11,18,22,26}) | | amide | |
| D220 | 2613.1 | Ac-KL(D-Dap)SLL(D-Dap)TLS(D-Dap)AKAAKL(D-Dap)TLL(D-Dap)ALS(D-Dap)- | 24 |
| (D-Dap^{3,7,11,18,22,26}) | | amide | |
| D221(L-Dap^{3,7,22,26}) | 2613.1 | Ac-KL(L-Dap)SLL(L-Dap)TLS(D-Dap)AKAAKL(D-Dap)TLL(L-Dap)ALS(L-Dap)- | 25 |
| (D-Dap^{11,18}) | | amide | |
| D222(L-Dap^{11,18} (D- | 2613.1 | Ac-KL(D-Dap)SLL(D-Dap)TLS(L-Dap)AKAAKL(L-Dap)TLL(D-Dap)ALS(D-Dap)- | 26 |
| Dap^{3,7,22,26}) | | amide | |
| D230 | 2614.1 | Ac-KL(L-Dap)SLL(L-Dap)TLS(L-Dap)AKAAKL(L-Dap)TLL(L-Dap)ALS(Ser)- | 27 |
| (L-Dap^{3,7,14,15,22})Ser²⁶ | | amide | |
| D231 | 2614.1 | Ac-KL(D-Dap)SLL(D-Dap)TLS(D-Dap)AKAAKL(D-Dap)TLL(D-Dap)ALS(Ser)- | 28 |
| (D-Dap^{3,7,14,15,22})Ser²⁶ | | amide | |
| Amino | −2 Dap | Lys residues at positions 1, 13 and 16 | |
| acid positions | Series | 1 3 7 14 15 22 26 | |
| D106 | 2613.1 | Ac-KL(L-Dap)SLL(L-Dap)TLSAA K(L-Dap)(L-Dap) KLATLL(L-Dap)ALS(L-Dap)- | 29 |
| (L-Dap^{3,7,14,15,22,26}) | | amide | |
| D240 | 2613.1 | Ac-KL(D-Dap)SLL(D-Dap)TLSAA K(D-Dap)(D-Dap) KLATLL(D-Dap)ALS(D-Dap)- | 30 |
| (D-Dap^{3,7,14,15,22,26}) | | amide | |
| D241(L-Dap^{3,7,22,26}) | 2613.1 | Ac-KL(L-Dap)SLL(L-Dap)TLSAA K(D-Dap)(D-Dap) KLATLL(L-Dap)ALS(L-Dap)- | 31 |
| (D-Dap^{14,15}) | | amide | |
| D242(L-Dap^{14,45}) | 2613.1 | Ac-KL(D-Dap)SLL(D-Dap)TLSAA K(L-Dap)(L-Dap) KLATLL(D-Dap)ALS(D-Dap)- | 32 |
| (D-Dap^{3,7,22,26}) | | amide | |

TABLE 1D
Non-polar face substitutions at amino acid positions 13 and 16

| Laboratory Peptide | | SEQ |
|---|---|---|
| Name | Sequence | ID |
| Amino acid positions | 1 3 7 13 14 15 16 22 26 | NO |
| D89-(Lys¹Lys¹³Lys¹⁶- | Ac-KL(L-Dab)SLL(L-Dab)TLSAA(Lys)(L-Dab)(L-Dab)(Lys)LATLL(L-Dab)ALS(L-Dab)- | 33 |
| 6Dab-2) | amide | |
| D118-(Lys¹Dab¹³Dab¹⁶- | Ac-KL(L-Dab)SLL(L-Dab)TLSAA(L-Dab)(L-Dab)(L-Dab)(L-Dab)LATLL(L-Dab)ALS(L-Dab)- | 34 |
| 6Dab-2) | amide | |
| D119-(Lys¹Dap¹³Dap¹⁶- | Ac-KL(L-Dab)SLL(L-Dab)TLSAA(L-Dap)(L-Dab)(L-Dab)(L-Dap)LATLL(L-Dab)ALS(L-Dab)- | 35 |
| 6Dab-2) | amide | |
| D89-(Lys¹A1a¹³Ala¹⁶- | Ac-KL(L-Dab)SLL(L-Dab)TLSAA(Ala)(L-Dab)(L-Dab)(Ala)LATLL(L-Dab)ALS(L-Dab)- | |
| 6Dab-2) | amide | |
| D500-(Lys¹Dab^{l3}Dab¹⁶- | Ac-KL(D-Dab)SLL(D-Dab)TLSAA(D-Dab)(D-Dab)(D-Dab)(D-Dab)LATLL(D-Dab)ALS(D-Dab)- | 36 |
| 6Dab-2) | amide | |
| D501-(Lys¹Dap^{l3}Dap¹⁶- | Ac-KL(D-Dab)SLL(D-Dab)TLSAA(D-Dap)(D-Dab)(D-Dab)(D-Dap)LATLL(D-Dab)ALS(D-Dab)- | 37 |
| 6Dab-2) | amide | |
| D502-(Lys¹Ala^{l3}Ala¹⁶- | Ac-KL(D-Dab)SLL(D-Dab)TLSAA(Ala)(D-Dab)(D-Dab)(Ala)LATLL(D-Dab)ALS(D-Dab)- | 45 |
| 6Dab-2) | amide | |

Another series of peptides (shown in Table 1D) were designed and tested to show the effects of changing the type of positively-charged residues at the position of the specificity determinants (positively charged residues at positions 13 and 16 in the center of the non-polar face of the 26-mer AMPs) on the non-polar face of the AMPs. In Table 1D, the ‘D-’ denotes that all amino acid residues in each peptide are in the D-enantiomeric conformation except for L-Dab and L-Dap residues, which are in the L-enantiomeric conformation. Peptide sequences are shown using the one-letter code for all amino acid residues except for L-Dap, L-Dab, and alanine (Ala) residues where the three-letter code is used. “Ac” denotes N^{α}-acetyl and amide denotes C^{α}-amide. Positions 13 and 16 are positively-charged residues (Lys, L-Dab, and L-Dap) or Ala residues on the non-polar face of the amphipathic α-helix.

Another series of peptides (shown in Table 1E) were designed and tested to show the effects of changing the location of L-Dab and L-Dap amino acid residues on the polar face of the AMPs. In Table 1E, the ‘D-’ denotes that all amino acid residues in each peptide are in the D-enantiomeric conformation except for L-Dab and L-Dap residues, which are in the L-enantiomeric conformation. Specificity determinants are positively charged residues in the center of the non-polar face of the 26-mer AMPs (i.e., Lys13 and Lys16). Peptide sequences are shown using the one-letter code for all amino acid residues except for L-Dap, L-Dab, lysine (Lys), and serine (Ser) residues where the three-letter code is used. “Ac” denotes N^{α}-acetyl and amide denotes C^{α}-amide. Positions 3, 7, 11, 14, 15, 18, 22, and 26 are positively charged residues (Lys, L-Dab and L-Dap) on the polar face of the amphipathic α-helix (see FIG. 1); −1 denotes 6 positively-charged residues on the polar face at positions 3, 7, 11, 18, 22 and 26 or 5 positively-charged residues on the polar face at positions 3, 7, 11, 18 and 22 (position 26 is substituted by Ser); −2 denotes 6 positively charged residues on the polar face at positions 3, 7, 14, 15, 22 and 26 or 5 positively charged residues on the polar face at positions 3, 7, 14, 15 and 22 (position 26 is substituted by Ser).

Another series of peptides (shown in Table 1F) were designed and tested to show the effects of changing the type of positively-charged amino acid residues on the polar face of the AMPs. In Table 1F, all amino acid residues are in the D-enantiomeric conformation except for L-Dab and L-Dap residues, which are in the L-enantiomeric conformation. Specificity determinants are positively charged residues in the center of the non-polar face of the 26-mer AMPs (i.e., Lys13 and Lys16). Peptide sequences are shown using the one-letter code for all amino acid residues except for Dap, Dab, lysine (Lys), arginine (Arg), and ornithine (Orn) residues where the three-letter code is used. Positions 1, 3, 7, 11, 18, 22, and 26 are positively-charged residues (Lys, L-Dab and L-Dap) on the polar face of the amphipathic α-helix (see FIG. 1); −1 denotes 6 positively-charged residues on the polar face at positions 3, 7, 11, 18, 22, and 26, or 5 positively-charged residues on the polar face at positions 3, 7, 11, 18 and 22 (position 26 is substituted by Ser).

TABLE 1E
Polar face substitutions of positively charged residues in AMPs

| Laboratory Peptide | Sequence | SEQ ID |
|---|---|---|
| Name | Specificity determinants (Lys13/Lys16) on non-polar face | NO |
| Amino acid positions | 1 3 7 11 18 22 26 | |
| D84(Lys¹-Lys-1) | Ac-KL(Lys)SLL(Lys)TLS(Lys)AKAAKL(Lys)TLL(Lys)ALS(Lys)-amide | 38 |
| D86(Lys¹-6Dab-1) | Ac-KL(L-Dab)SLL(L-Dab)TLS(L-Dab)AKAAKL(L-Dab)TLL(L-Dab)ALS(L-Dab)- | 2 |
| | amide | |
| D105(Lys¹-6Dap-1) | Ac-KL(L-Dap)SLL(L-Dap)TLS(L-Dap)AKAAKL(L-Dap)TLL(L-Dap)ALS(L-Dap)- | 23 |
| | amide | |
| D101(Lys¹Ser²⁶-5Lys-1) | Ac-KL(Lys)SLL(Lys)TLS(Lys)AKAAKL(Lys)TLL(Lys)ALS(Ser)-amide | 39 |
| D102(Lys¹Ser²⁶-5Dab-1) | Ac-KL(L-Dab)SLL(L-Dab)TLS(L-Dab)AKAAKL(L-Dab)TLL(L-Dab)ALS(Ser)- | 7 |
| | amide | |
| Amino acid positions | 1 3 7 14 15 22 26 | |
| D88(Lys¹-6Lys-2) | Ac-KL(Lys)SLL(Lys)TLSAAK(Lys)(Lys)KLATLL(Lys)ALS(Lys)-amide | 40 |
| D89(Lys¹-6Dab-2) | Ac-KL(L-Dab)SLL(L-Dab)TLSAAK(L-Dab)(L-Dab)KLATLL(L-Dab)ALS(L-Dab)- | 41 |
| | amide | |
| D106(Lys¹-6Dap-2) | Ac-KL(L-Dap)SLL(L-Dap)TLSAAK(L-Dap)(L-Dap)KLATLL(L-Dap)ALS(L-Dap)- | 29 |
| | amide | |
| D103(Lys¹Ser²⁶-5Lys-2) | Ac-KL(Lys)SLL(Lys)TLSAAK(Lys)(Lys)KLATLL(Lys)ALS(Ser)-amide | 42 |
| D104(Lys¹Ser²⁶-5Dab-2) | Ac-KL(L-Dab)SLL(L-Dab)TLSAAK(L-Dab)(L-Dab)KLATLL(L-Dab)ALS(Ser)- | 11 |
| | amide | |

TABLE 1F
Polar face substitutions of positively charged residues in AMPs.

| | | SEQ |
|---|---|---|
| Laboratory Peptide | | ID |
| Name | Sequence | NO |
| | With specificity determinants (Lys13/Lys16) | |
| Amino acid positions | 1 3 7 11 18 22 26 | |
| D87(Lys¹-6Arg-1) | Ac-KL(Arg)SLL(Arg)TLS(Arg)AKAAKL(Arg)TLL(Arg)ALS(Arg)-amide | 43 |
| D84(Lys¹-6Lys-1) | Ac-KL(Lys)SLL(Lys)TLS(Lys)AKAAKL(Lys)TLL(Lys)ALS(Lys)-amide | 38 |
| D85(Lys¹-6Orn-1) | Ac-KL(Orn)SLL(Orn)TLS(Orn)AKAAKL(Orn)TLL(Orn)ALS(Orn)-amide | 44 |
| D86(Lys¹-6Dab-1) | Ac-KL(Dab)SLL(Dab)TLS(Dab)AKAAKL(Dab)TLL(Dab)ALS(Dab)-amide | 2 |
| D105(Lys¹-6Dap-1) | Ac-KL(Dap)SLL(Dap)TLS(Dap)AKAAKL(Dap)TLL(Dap)ALS(Dap)-amide | 23 |
| D101(Lys¹Ser²⁶-5Lys-1) | Ac-KL(Lys)SLL(Lys)TLS(Lys)AKAAKL(Lys)TLL(Lys)ALS S-amide | 39 |
| D102(Lys¹Ser²⁶-5Dab- | Ac-KL(Dab)SLL(Dab)TLS(Dab)AKAAKL(Dab)TLL(Dab)ALS S-amide | 7 |
| 1) | | |
| | Without specificity determinants (Ala13/Ala16) | |
| Amino acid positions | 1 3 7 11 18 22 26 | |
| D85(K13A/K16A)- | Ac-KL(Orn)SLL(Orn)TLS(Orn)AAAAAL(Orn)TLL(Orn)ALS(Orn)-amide | 46 |
| (Lys¹-6Orn-1) | | |
| D86(K13A/K16A)- | Ac-KL(Dab)SLL(Dab)TLS(Dab)AAAAAL(Dab)TLL(Dab)ALS(Dab)-amide | 47 |
| (Lys¹-6Dab-1) | | |
| D105(K13A/K16A)- | Ac-KL(Dap)SLL(Dap)TLS(Dap)AAAAAL(Dap)TLL(Dap)ALS(Dap)-amide | 48 |
| (Lys¹-6Dap-1) | | |

Compositions of this Disclosure

When employed as pharmaceuticals, especially as antimicrobial agents administered to mammals, the AMPs of this disclosure are administered in the form of pharmaceutical compositions. These compounds can be administered by a variety of routes including oral, rectal, transdermal, subcutaneous, intravenous, intramuscular, intrathecal, and intranasal. Such pharmaceutical compositions are prepared in a manner well known in the pharmaceutical art and comprise at least one AMP of this disclosure.

The pharmaceutical compositions of the present invention contain, as the active ingredient, one or more of the AMPs of this disclosure, associated with pharmaceutically acceptable formulations. In making the compositions of this invention, the AMP active ingredient is usually mixed with an excipient, diluted by an excipient, or enclosed within a carrier which can be in the form of a capsule, sachet, paper, or other container. An excipient is usually an inert substance that forms a vehicle for a drug. When the excipient serves as a diluent, it can be a solid, semi-solid, or liquid material, which acts as a vehicle, carrier or medium for the active ingredient. Thus, the compositions can be in the form of solutions, syrups, aerosols (as a solid or in a liquid medium), ointments containing, for example, up to 30% by weight of the active compound, soft and hard gelatin capsules, suppositories, sterile injectable solutions, and sterile packaged powders.

In preparing a formulation, it may be necessary to mill active compounds of this disclosure to provide the appropriate particle size prior to combining with the other ingredients. If the antimicrobial peptide is substantially insoluble, it ordinarily is milled to a particle size of less than 200 mesh. If the compound(s) is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, gum Arabic, calcium phosphate, alginates, tragacanth, gelatin, calcium silicate, microcrystalline cellulose, polyvinylpyrrolidone, cellulose, sterile water, syrup, and methylcellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of this disclosure can be formulated to provide quick, sustained or delayed release of the active ingredient after administration to the patient by employing procedures known in the art.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active AMP(s).

Formulations of this disclosure suitable for oral administration may be in the form of capsules, cachets, pills, tablets, powders, granules or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of a compound or compounds of the present invention as an active ingredient. A compound or compounds of the present invention may also be administered as bolus, electuary or paste.

In solid dosage forms of this disclosure for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

A tablet may be made by compression or molding optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may release the active ingredient only, or preferentially, in a certain portion of the gastrointestinal tract, optionally, in a delayed manner. Examples of embedding compositions which can be used include polymeric substances and waxes. The active ingredient can also be in microencapsulated form.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials may be used for such enteric layers or coatings, including a number of polymeric acids and mixtures of polymeric acids with such materials as shellac, cetyl alcohol, and cellulose acetate.

Liquid dosage forms for oral administration of the compounds of this disclosure include pharmaceutically-acceptable emulsions, microemulsions, solutions, suspensions, syrups and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (e.g., cottonseed, groundnut, corn, germ, olive, castor, sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents, such as ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of this disclosure for rectal or vaginal administration may be presented as a suppository, which may be prepared by mixing one or more compounds of this disclosure with one or more suitable nonirritating excipients or carriers comprising, for example, cocoa butter, polyethylene glycol, a suppository wax or salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration of compounds of this invention include powders, sprays, ointments, pastes, creams, lotions, gels, solutions, patches, drops and inhalants. The active AMP(s) may be mixed under sterile conditions with a pharmaceutically-acceptable carrier, and with buffers or propellants that may be required.

The ointments, pastes, creams and gels may contain, in addition to an AMP active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an AMP active ingredient, excipients such as lactose, talc, silicic acid, aluminum hydroxide, calcium silicates and polyamide powder or mixtures of these substances. Sprays may also contain customary propellants such as chlorofluorohydrocarbons and volatile unsubstituted hydrocarbons, such as butane and propane.

Transdermal patches have the added advantage of providing controlled delivery of AMPs of this disclosure to the body. Such dosage forms can be made by dissolving, dispersing or otherwise incorporating one or more compounds of this disclosure in a proper medium, such as an elastomeric matrix material. Absorption enhancers can also be used to increase the flux of the compound across the skin. The rate of such flux can be controlled by providing a rate-controlling membrane or dispersing a compound in a polymer matrix or gel.

Pharmaceutical formulations include those suitable for administration by inhalation or insufflation or for nasal or intraocular administration. For administration to the upper (nasal) or lower respiratory tract by inhalation, the compounds of this disclosure are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of one or more compounds of this disclosure and a suitable powder base, such as lactose or starch. The powder composition may be presented in unit dosage form in, for example, capsules or cartridges, or, e.g., gelatin or blister packs from which the powder may be administered with the aid of an inhalator, insufflator or a metered-dose inhaler.

For intranasal administration, compounds of this disclosure may be administered by means of nose drops or a liquid spray such as a plastic bottle atomizer or metered-dose inhaler. Typical of atomizers are the Mistometer (Wintrop) and Medihaler (Riker).

Drops, such as eye drops or nose drops, may be formulated with an aqueous or nonaqueous base also comprising one or more dispersing agents, solubilizing agents or suspending agents. Liquid sprays are conveniently delivered from pressurized packs. Drops can be delivered by means of a simple eye dropper-capped bottle or by means of a plastic bottle adapted to deliver liquid contents dropwise by means of a specially shaped closure.

Pharmaceutical compositions of this invention suitable for parenteral administration comprise one or more AMP of this disclosure in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and nonaqueous carriers which may be employed in the pharmaceutical compositions of this disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monosterate and gelatin.

In some cases, to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug is accomplished by dissolving or suspending the drug in an oil vehicle.

Injectable depot forms are made by forming microencapsule matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue. The injectable materials can be sterilized for example, by filtration through a bacterial-retaining filter.

These formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

Suitable alkalinizing agents include alkali metal salts and alkaline earth metal salts. The alkali metal salts include sodium carbonate, sodium hydroxide, sodium silicate, disodium hydrogen orthophosphate, sodium aluminate, and other suitable alkali metal salts or mixtures thereof. Suitable alkaline metal salts include calcium carbonate, calcium hydroxide, magnesium carbonate, magnesium hydroxide, magnesium silicate, magnesium aluminate, aluminum magnesium hydroxide or mixtures thereof. More particularly, calcium carbonate, potassium bicarbonate, calcium hydroxide, and/or sodium carbonate may be used as alkalinizing agents to obtain a formulation pH within the desired pH range of pH 8 to pH 13. The concentration of the alkalinizing agent is selected to obtain the desired pH, varying from about 0.1% to about 30%, by weight, and more preferably from about 12.5% to about 30%, by weight, of the total weight of the dosage formulation.

Suitable antioxidants may be selected from amongst one or more pharmaceutically acceptable antioxidants known in the art. Examples of pharmaceutically acceptable antioxidants include butylated hydroxyanisole (BHA), sodium ascorbate, butylated hydroxytoluene (BHT), sodium sulfite, citric acid, malic acid and ascorbic acid. Antioxidants may be present in these formulations at a concentration between about 0.001% to about 5%, by weight, of the dosage formulation.

Suitable chelating agents may be selected from amongst one or more chelating agents known in the art. Examples of suitable chelating agents include disodium edetate (EDTA), edetic acid, citric acid and combinations thereof. The chelating agents may be present in a concentration between about 0.001% and about 5%, by weight, of the dosage formulation.

### Methods for Preventing and Treating Microbial Infection

Another aspect of this disclosure provides methods for preventing and treating a microbial infection. These methods include administering to a subject in need thereof a therapeutically effective amount of a peptide or composition of this disclosure that kills or inhibits the growth of infectious microbes, thereby inhibiting or treating the microbial infections. The infecting microorganism may include Gram-negative bacteria. Gram-negative bacteria may include, but are not limited to, Escherichia coli, Pseudomonas aeruginosa, Acinetobacter baumannii, Salmonella spp., Haemophilus influenzae, Neisseria spp., Vibrio cholerae, Vibrio parahaemolyticus and Helicobacter pylori.

The antimicrobial peptides administered, preferably as a component of a pharmaceutical composition, can include a single antimicrobial peptide of this disclosure, or multiple peptides of this disclosure. The peptides may include peptides having at least 84%, or at least 88%, or at least 92% amino acid sequence homology to a peptide sequence of SEQ ID NOs:1-44, and which effectively treat or prevent a microbial infection. Thus, the peptides may include 26-mer peptides having 1, 2, 3, or 4 individual amino acid changes in a peptide sequence of any one of SEQ ID NOs:1-44. The peptides may include fragments of the peptides of SEQ ID NOs:1-44 that retain the ability to effectively treat or prevent a microbial infection. Exemplary peptides include the amino acid sequences set forth in SEQ ID NOs: 2-32, 34-37, and 41.

Therapeutic AMPs of this disclosure may be administered by a number of routes, including orally, topically, or parenteral administration, including for example, intravenous by injection or infusion, intraperitoneal, intramuscular, intradermal, intrathecal, intrasternal, intraarticular, or subcutaneous injection. One of skill in the art can determine the appropriate route of administration.

The therapeutically effective amounts of the AMPs of this disclosure that inhibit or kill an infecting microorganism will depend upon the subject being treated, the severity and type of the infection, and the manner of administration. For example, a therapeutically effective amount of a peptide of this disclosure can vary from about 1 microgram/injection up to about 10 mg/injection. The exact amount of the peptide is readily determined by one of skill in the art based on the age, weight, sex, and physiological condition of the subject. Effective doses can be extrapolated from dose-response curves derived from in vitro or animal model test systems.

One or more peptides of this disclosure that effectively inhibit or kill an infecting microorganism can be administered in conjunction with one or more additional pharmaceutical agents. The additional pharmaceutical agents can be administered at the same time as, or sequentially with, the peptide(s) of this disclosure. The additional pharmaceutical agent may be an additional antimicrobial agent. When administered at the same time, the additional pharmaceutical agent(s) can be formulated in the same composition that includes the peptide(s) of this disclosure.

Those skilled in the art can determine an appropriate time and duration of therapy that includes the administration of a peptide of this disclosure to achieve the desired preventative or ameliorative effects on the subject treated.

The invention now being generally described will be more readily understood by reference to the following examples, which are included merely for the purposes of illustration of certain aspects of the invention. The examples are not intended to limit the invention, as one of skill in the art would recognize from the above teachings and the following examples that other techniques and methods can satisfy the claims and can be employed without departing from the scope of the invention.

### EXAMPLES

The following methods were used to conduct the experiments described in Examples 1-8, below: Solid-phase Peptide Synthesis: Standard solid-phase peptide synthesis methodology using 9-fluorenylmethoxycarbonyl (Fmoc) chemistry and Fmoc-rink amide 4-methylbenzhydrylamine resin (P3 BioSystems, Louisville, Ky.) (substitution 0.65 mmol/g) using a Focus-XC peptide synthesizer (Aapptec, Louisville, Ky.). The coupling procedure used 5 equivalents of Fmoc-D-amino acid or Fmoc-L-amino acid and HCTU (O-(1H-6-chlorobenzotriazole-1-yl)-1,1,3,3-tetramethyluronium hexafluorophosphate) and 6-Cl-HOBt (1-hydroxy-6-chloro-benztriazole) at 4.5 equivalents in DMF (Fisher Scientific, Denver, Colo.) and N-diisopropylethylamine (DIPEA) 10 equivalents (Oakwood Products, Inc., Estill, S.C.) in N-methyl-2-pyrrolidinone (NMP) (Fisher Scientific, Denver, Colo.) with the first and second couplings at room temperature for 30 min each. The deprotection procedure (removal of Fmoc protecting group) was carried out by treatment of the resin with 0.1 M HOBt (1-hydroxybenzotriazole) in DMF with 20% piperidine for 30 min. After completion of the synthesis, the peptide resin was dried under vacuum and the peptide was cleaved from the resin with a mixture of 94% trifluoroacetic acid (TFA), 2.5% water, 2.5% 1,2-ethanedithiol (EDT) and 1% triisopropylsilane (TIS) for 2 h. The resin was removed by filtration and peptide was precipitated with ice-cooled ethyl ether on ice for 15 min. Ether was decanted, the peptide was washed twice with ether and redissolved in acetonitrile/water (1:1, with 0.2% TFA) and the solution lyophilized to obtain the crude peptide.
Analytical and Preparative Purification by Reversed-Phase Chromatography: Analytical RP-HPLC: Column, Luna C18 (2), 250×4.6 mm I.D., 5 μm particle size, 100 Å pore size from Phenomenex (Torrance, Calif.). Run conditions: linear AB gradient (1% acetonitrile/min, starting from 2% acetonitrile) at a flow-rate of 1 ml/min, where eluent A is 0.2% aq. TFA and eluent B is 0.18% TFA in acetonitrile; temperature, 30° C. Preparative RP-HPLC: Peptides were dissolved in 0.2% aq. TFA containing 2% acetonitrile to a final concentration of 10 mg/ml. Following filtration through a 0.45 μm Millipore filter and subsequently through a 0.22 μm filter, the peptide solutions were loaded onto the column via multiple 20-ml injections into a 20-ml injection loop at a flow-rate of 10 ml/min. Column, Luna C18 (2), 250×30 mm I.D., 5 um particle size, 100 Å pore size from Phenomenex. Run conditions: 2% acetonitrile/min gradient up to an acetonitrile concentration 10% below that required to elute the peptide during analytical RP-HPLC, then shallow gradient elution (0.05% or 0.1% acetonitrile/min depending on the complexity of impurities near the product of interest on the analytical run of the crude peptide) at a flow-rate of 10 ml/min (same eluents as shown above for analytical RP-HPLC); at room temperature.
Temperature Profiling of Peptides on Reversed-phase HPLC to Measure Dimerization and Oligomerization: Purified peptides were analyzed on an Agilent 1200 series liquid chromatograph for temperature profiling using a Zorbax 300 SB-C8 column (150 mm×2.1 mm I.D.; 5 μm particle size, 300 Å pore size) from Agilent Technologies. Conditions: linear AB gradient (0.5% acetonitrile/min) and a flow rate of 0.30 ml/min, where eluent A was 0.20% aqueous TFA, pH 2 and eluent B was 0.18% TFA in acetonitrile. Temperature profiling was carried out on peptide mixtures run at each temperature in 2° C. increments from 5° C. to 41° C. and 10° C. increments from 5° C. to 75° C. Twenty minutes was allowed between runs for temperature equilibration.
Characterization of Helical Structure: The mean residue molar ellipticities of peptides were determined by circular dichroism (CD) spectroscopy, using a Jasco J-815 spectropolarimeter (Jasco Inc. Easton, Md., USA) at 5° C. under KP buffer (50 mM NaH₂PO₄/Na₂HPO₄/100 mM KCl, pH 7.0) as well as in the presence of an α-helix inducing solvent, 2,2,2-trifluoroethanol, TFE, (50 mM NaH₂PO₄/Na₂HPO₄/100 mM KCl, pH 7.0 buffer/50% TFE). A 10-fold dilution of an approximately 500 μM stock solution of the peptide analogs was loaded into a 0.1 cm quartz cell and its ellipticity scanned from 195 to 250 nm. Peptide concentrations were determined by amino acid analysis.
Determination of Peptide Amphipathicity: Amphipathicity of peptides at pH 2 was determined by the calculation of hydrophobic moment⁵⁹, using the software package EMBOSS 6.5.7 and the Hmoment application, modified to include hydrophobicity scales determined in our laboratory^{60,61}. The hydrophobicity scale used in this study is listed as follows: At pH 2, these coefficients were determined in 20 mM trifluoroacetic acid (TFA), Trp, 32.4; Phe, 29.1; Leu, 23.3; Ile, 21.4; Met, 15.7; Tyr, 14.7; Val, 13.4; Pro, 9.0; Cys, 7.6; Ala, 2.8; Glu, 2.8; Thr, 2.3; Asp, 1.6; Gln, 0.6; Ser, 0.0; Asn, −0.6; Gly, 0.0; Arg. 0.6; His, 0.0; Lys, 2.8; Orn, 2.1; Dab, −1.2; and Dap, 1.0 (polar face), Lys, −18.48 (center of non-polar face). This HPLC-derived scale reflects the relative difference in hydophilicity/hydrophobicity of the 20 amino acid side-chains more accurately than previously determined scales (see recent review where this scale was compared to other scales⁶¹). The hydrophobicity/hydrophilicity coefficients for Lys residues in the center of the non-polar face at pH 2.0 were assigned values of −18.48 determined by reversed-phase chromatography of the identical peptides where Ala was substituted by Lys on the non-polar face at positions 13 and 16. Position X was placed in the sequence where these values are to be used in the Hmoment calculations when Lys is in the center of the non-polar face. J, B and Z were used to denote Orn, Dab, and Dap, respectively.
Amino Acid Analysis for Peptide Quantitation: Amino acid analysis was performed according to the method described by Cohen and Michaud (Anal Biochem. 1993, 211, 279-87). Briefly, 20 μL of each peptide sample was aliquoted into glass tubes and lyophilized. To these tubes, 30 μL of 6M HCl w/0.1% phenol was added and the resulting solution was heated to 110° C. for 48 h in order to hydrolyze the peptide bonds in the sample. Each sample tube was allowed to come to room temperature and then vacuum-dried to remove the HCl. Each sample was then re-suspended in 10 mM HCl and 20 μL of the sample was added to 60 μL of 0.2M sodium borate buffer, pH 8.8. To this mixture, 20 μL of 6-aminoquinoyl-N-hydroxysuccinimidyl carbamate in acetonitrile was added to derivatize the amino acids present in the sample. After this addition, the derivatized sample was heated to 55° C. for 15 min to convert Tyr byproducts to one form. HPLC using an Agilent 1260 series instrument and a Waters AccQ Tag 3.9×150 mm column was used to separate and quantify the derivatized amino acids present in each sample. Quantification was by UV absorbance at 254 nm.
Gram-Negative Bacteria Strains used in this Study: The A. baumannii strains used in this study consisted of seven strains obtained from MERCK (M89941, M89949, M89951, M89952, M89953, M89955 and M89963). These seven A. baumannii strains were resistant to Polymyxin B and Colistin. In addition, we obtained 20 A. baumannii strains from JMI Laboratories, North liberty, Iowa, 2017/2018 world-wide isolates with resistance to antibiotics. These isolates came from four continents (Asia-W. Pacific (collection number 965463, 981650, 1035794, 1018887), Europe (963618, 963659, 964304, 968886, 1017395, 1010245, 1010282, 1035166), Latin America (977751, 1002956), and North America (961997, 9383370, 952654, 1021371, 1007660, 1001611), 12 different countries and 17 different cities. These isolates were screened against 18 different antibiotics (Amikacin (17/20), Ampicillin-sulbactam (17/20), Aztreonam (20/20), Cefepime (18/20), Ceftazidime (17/20), Ceftazidime-avibactam (17/20), Ciprofloxacin (15/20), Colistin (10/20), Doripenem (17/20), Doxycycline (13/17), Gentamicin (18/20), Imipenem (17/20), Levofloxacin (14/20), Meropenem (17/20), Minocycline (13/17), Piperacillin-tazobactam (19/20), Tetracycline (15/17) and Tobramycin (18/20). The values in brackets show the number of resistant isolates for that antibiotic out of the number of isolates screened. The blood strain 649 of A. baumannil used to determine the antimicrobial activity in the presence of human serum was obtained from the collection of Dr. Anthony A. Campagnari at the University of Buffalo.
Measurement of Antimicrobial Activity (MIC): The minimal inhibitory concentration (MIC) is defined as the lowest peptide concentration that inhibited bacterial growth. MICs were measured by a standard microtiter dilution method in Mueller Hinton (MH) medium. Briefly, cells were grown overnight at 37° C. in MH broth and were diluted in the same medium. Serial dilutions of the peptides were added to the microtiter plates in a volume of 50 μL, followed by the addition of 50 μL of bacteria to give a final inoculum of 5×10⁵ colony-forming units (CFU)/mL. The plates were incubated at 37° C. for 24 h, and the MICs were determined.
Measurement of Hemolytic Activity: Peptide samples (concentrations determined by amino acid analysis) were added to 1% human erythrocytes in phosphate-buffered saline (100 mM NaCl, 80 mM Na₂HPO₄, 20 mM NaH₂PO₄, pH 7.4) and the reaction mixtures were incubated at 37° C. for 18 h in microtiter plates. Two-fold serial dilutions of the peptide samples were carried out. This determination was made by withdrawing aliquots from the hemolysis assays and removing unlysed erythrocytes by centrifugation (800×g). Hemoglobin release was determined spectrophotometrically at 570 nm. The control for 100% hemolysis was a sample of erythrocytes treated with water. The control for no release of hemoglobin was a sample of 1% erythrocytes without any peptide added. Since erythrocytes were in an isotonic medium, no detectable release (<1% of that released upon complete hemolysis) of hemoglobin was observed from this control during the course of the assay. The hemolytic activity is generally determined as the peptide concentration that causes 50% hemolysis of erythrocytes after 18 h (HC₅₀). HC₅₀ was determined from a plot of percent lysis versus peptide concentration (μM). Hemolysis data is determined at 12 different concentrations up to 1000 micrograms per ml for 18 h at 37° C. The average of 3 determinations is used with an average variance of less than 4%. Fresh human blood was obtained from Vitalant, Denver, Colo., USA.
Calculation of Therapeutic Index: The therapeutic index is a widely accepted parameter to represent the specificity of antimicrobial peptides for prokaryotic versus eukaryotic cells. It is calculated by the ratio of hemolytic activity and antimicrobial activity (MIC_{GM} (geometric mean MIC value)); thus, larger values of therapeutic index indicate greater specificity for prokaryotic cells. With the peptides used in this study we used the HC₅₀/MIC ratio value to calculate the therapeutic index (T.I.).

### Example 1

### Peptide Design, Specificity Determinants, and Positively-Charged Residue on the Polar Face

Enantiomeric forms of AMPs with all-D-amino acids have shown equal activities to their all-L-enantiomers. The advantage of all-D-peptides is that they are resistant to proteolytic enzyme degradation, which enhances their potential as therapeutic agents. In this study, we designed de novo, synthesized, purified and characterized ten potentially amphipathic α-helical antimicrobial peptides (AMPs). Seven AMPs have “specificity determinants”, lysine residues on the non-polar face at positions 13 and 16, and three AMPs are without “specificity determinants,” where the lysine residues at positions 13 and 16 were replaced with alanine residues, as described in Table 1G.

Five AMPs have six positively-charged residues on the polar face which contain Arg, Lys, Orn, Dab, or Dap residues at positions 3, 7, 11, 18, 22 and 26; two AMPs have only five positively charged residues on the polar face at positions 3, 7, 11, 18 and 22, containing either five Lys or five Dab residues (position 26 has been replaced with Ser) (Table 1G). All ten peptides have a lysine residue at position 1 and the net charges on these peptides are either +9 or +8 for the AMPs with “specificity determinants” or +7 for the AMPs without “specificity determinants” (Table 1G).

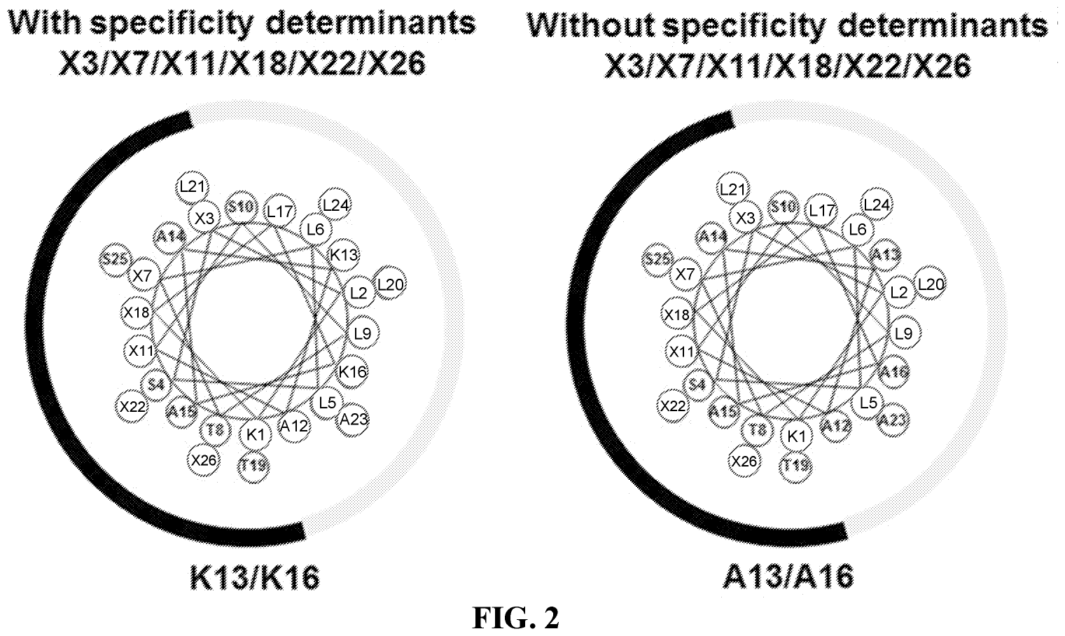

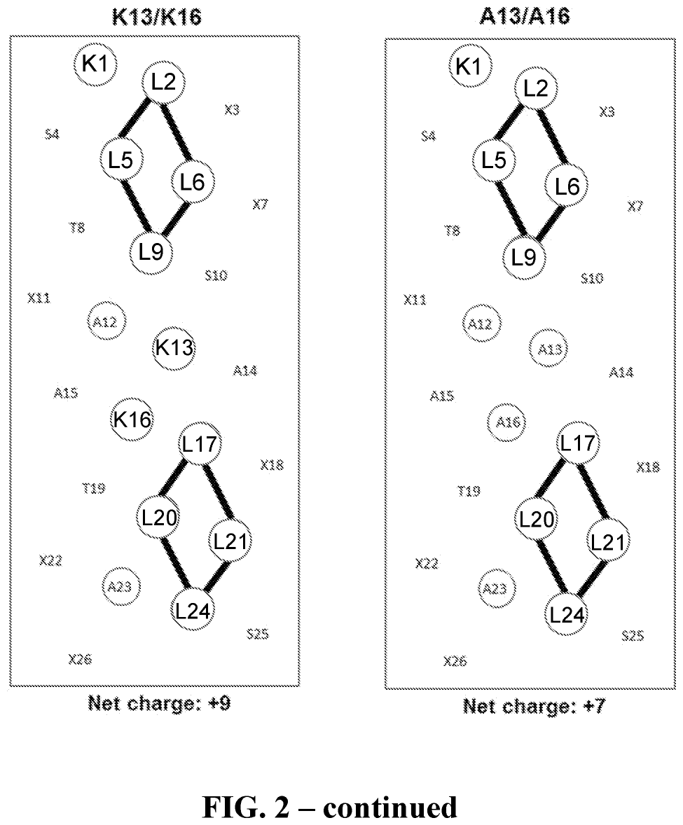

FIG. 1 shows a general amino acid sequence in a helical wheel and helical net representations where X₃X₇X₁₁X₁₈X₂₂X₂₆ show the positions on the polar face of the positively-charged residues. We have displayed two versions of the helical nets where the polar residues are displayed along the center of the helical net (left side) and where the non-polar residues are displayed along the center of the helical net (right side). The hydrophobic/non-polar faces of the seven peptides with “specificity determinants” have eight Leu residues in two clusters of four separated by the two Lys residues (“specificity determinants” in the center of the non-polar face). Lys 1 is also on the non-polar face. FIG. 2 shows the difference between the peptides with “specificity determinants” (Lys 13 and Lys 16) in the center of the non-polar face and those without (Ala 13 and Ala 16). Thus, the positive charge on the non-polar face decreases from +3 to +1 and the overall net charge on the AMPs decreases from +9 to +7.

TABLE 1G
Polar face substitutions of positively-charged residues in AMPs.

| Peptide | Net | | SEQ ID |
|---|---|---|---|
| Name^{a} | Charge | Sequence^{b} | NO |
| Amino Acid | | With specificity determinants (Lys13/Lys16) | |
| positions | | 1 3 7 11 18 22 26 | |
| D87(Lys¹-6Arg-1) | +9 | Ac-KL(Arg)SLL(Arg)TLS(Arg)AKAAKL(Arg)TLL(Arg)ALS(Arg)-amide | 43 |
| D84(Lys¹-6Lys-1) | +9 | Ac-KL(Lys)SLL(Lys)TLS(Lys)AKAAKL(Lys)TLL(Lys)ALS(Lys)-amide | 38 |
| D85(Lys¹-6Orn-1) | +9 | Ac-KL(Orn)SLL(Orn)TLS(Orn)AKAAKL(Orn)TLL(Orn)ALS(Orn)-amide | 44 |
| D86(Lys¹-6Dab-1) | +9 | Ac-KL(Dab)SLL(Dab)TLS(Dab)AKAAKL(Dab)TLL(Dab)ALS(Dab)-amide | 2 |
| D105(Lys¹-6Dap-1) | +9 | Ac-KL(Dap)SLL(Dap)TLS(Dap)AKAAKL(Dap)TLL(Dap)ALS(Dap)-amide | 23 |
| D101(Lys¹Ser²⁶-5 | +8 | Ac-KL(Lys)SLL(Lys)TLS(Lys)AKAAKL(Lys)TLL(Lys)ALSS-amide | 39 |
| Lys-1) | | | |
| D102(Lys¹Ser²⁶- | +8 | Ac-KL(Dab)SLL(Dab)TLS(Dab)AKAAKL(Dab)TLL(Dab)ALSS-amide | 7 |
| 5Dab-1) | | | |
| Amino Acid | | Without specificity determinants (Ala13/Ala16) | |
| positions | | 1 3 7 11 18 22 26 | |
| D85(K13A/K16A)- | +7 | Ac-KL(Orn)SLL(Orn)TLS(Orn)AAAAAL(Orn)TLL(Orn)ALS(Orn)-amide | 46 |
| (Lys¹-6Orn-1) | | | |
| D86(K13A/K16A)- | +7 | Ac-KL(Dab)SLL(Dab)TLS(Dab)AAAAAL(Dab)TLL(Dab)ALS(Dab)-amide | 47 |
| (Lys¹-6Dab-1) | | | |
| D105(K13A/K16A)- | +7 | Ac-KL(Dap)SLL(Dap)TLS(Dap)AAAAAL(Dap)TLL(Dap)ALS(Dap)-amide | 48 |
| (Lys¹-6Dap-1) | | | |

^{a}The D denotes that all amino acid residues in each peptide are in the D-conformation. Specificity determinants are positively-charged residues in the center of the non-polar face (Lys13/Lys16). “Without specificity determinants” means replacement with Ala residues (Ala13/Ala16) (FIG. 2).
^{b}Peptide sequences are shown using the one-letter code for all amino acid residues except at X¹, X², X³, X⁴, X⁵ and (except for D101 and D102) X⁶, where the three-letter code is used. Ac denotes N^{α}-acetyl and amide denotes C^{α}-amide. Positions X¹, X², X³, X⁴, X⁵ and (except for D101 and D102) X⁶ are positively charged residues (Arg, Lys, Orn, Dab and Dap) on the polar face of the amphipathic α-helix (FIG. 1); −1 denotes 6 positively charged residues on the polar face at positions 3, 7, 11, 18, 22, and 26 or 5 positively charged residues on the polar face at positions 3, 7, 11, 18, and 22 (position 26 is substituted by Ser).

### Example 2

### Antibacterial Activity

Table 2 shows the antibacterial activities against 7 different Acinetobacter baumannii strains resistant to polymyxin B and colistin (antibiotics of last resort).

The geometric mean MIC values for the five AMPs, where the positively-charged residue was varied from Arg, Lys, Orn, Dab and Dap, ranged from 0.5 μM (6 Lys and 6 Orn containing peptides) to 0.8 μM for 6 Arg, 1.0 μM for 6 Dab and 1.2 μM for 6 Dap residues on the polar face. Thus, shortening the number of carbon atoms between the side-chain amino group and the α-carbon atom from four (Lys) to one (diaminopropionic acid) had very little effect on antibacterial activity (Table 2). We also determined the antibacterial activity of our five AMPs against 20 additional Acinetobacter baumannii isolates from four continents, 12 different countries and 17 different cities that were resistant to 18 different antibiotics. As shown in Table 3, the geometric mean MIC value was 0.7 μM for the 6 Lys-containing AMP to 1.0 μM for 6 Dab- or 6 Dap-containing AMPs. Thus, it is very clear that changing the type of positively-charged residue on the polar face of these AMPs had very little effect on the antibacterial activities. Removing the C-terminal positively charged residue from the peptides D84(Lys26Ser) and D86(Dab26Ser) to give peptides D101 and D102 had a small effect or actually enhanced the geometric mean MIC value (D84 0.5 μM vs D101 0.8 μM) and (D86 1.0 μM vs D102 0.7 μM) (Table 2).

TABLE 2
Antibacterial activity against 7 strains of Acinetobacter baumannii resistant to
Polymyxin B and Colistin

| | | MIC | | | | | | | |
|---|---|---|---|---|---|---|---|---|---|
| | Peptide | (μm)^{b} | | | | | | | MIC_{GM} |
| Peptide Name^{a} | Mass | MB9941 | MB9949 | MB9951 | MB9952 | MB9953 | MB9955 | MB9963 | (μm)^{b} |
| With specificity determinants | | | | | | | | | |
| D87(Lys¹-6Arg-1) | 3033.7 | 1.3 | 1.3 | 0.7 | 0.7 | 0.7 | 0.7 | 0.7 | 0.8 |
| D84(Lys¹-6Lys-1) | 2865.6 | 0.3 | 0.7 | 0.3 | 0.7 | 0.7 | 0.3 | 0.7 | 0.5 |
| D85(Lys¹-6Orn-1) | 2781.5 | 0.7 | 0.7 | 0.7 | 0.4 | 0.4 | 0.4 | 0.4 | 0.5 |
| D86(Lys¹-6Dab-1)\grave{ } | 2697.3 | 0.7 | 0.7 | 1.4 | 0.7 | 1.4 | 0.7 | 1.4 | 1.0 |
| D105(Lys¹-6Dap-1) | 2613.1 | 0.8 | 0.8 | 3.0 | 0.8 | 3.0 | 0.8 | 1.5 | 1.2 |
| D101(Lys¹Ser²⁶-5Lys-1) | 2824.5 | 0.7 | 0.7 | 0.7 | 0.7 | 1.5 | 0.7 | 0.7 | 0.8 |
| D102(Lys¹Ser²⁶-5Dab-1) | 2684.3 | 0.4 | 0.7 | 0.7 | 0.7 | 1.4 | 0.7 | 0.7 | 0.7 |
| Without | | | | | | | | | |
| specificity determinants | | | | | | | | | |
| D85(K13A/K16A)-(Lys¹-6Orn-1) | 2667.3 | 2.9 | 2.9 | 1.5 | 1.5 | 1.5 | 1.5 | 2.9 | 2.0 |
| D86(K13A/K16A)-(Lys¹-6Dab-1) | 2583.1 | 1.5 | 0.8 | 0.8 | 0.8 | 0.8 | 0.8 | 0.8 | 0.9 |
| D105(K13A/K16A)-(Lys¹-6Dap-1) | 2499.0 | 1.6 | 0.8 | 0.8 | 1.6 | 1.6 | 1.6 | 1.6 | 1.3 |
| Colistin | 1155.5 | >28 | >28 | >28 | >28 | >28 | >28 | >28 | >28 |
| Polymyxin B | 1301.6 | >25 | >25 | >25 | >25 | >25 | >25 | >25 | >25 |

^{a}6Lys−1 denotes 6Lys residues on the polar face at positions 3, 7, 11, 18, 22, and 26; 5Lys−1 denotes 5Lys residues of polar face at positions 3, 7, and 22 (position 26 is substituted by Ser).
bMIC is minimal inhibitory concentration (μM) that inhibited growth of different strains in Mueller-Hinton (MH) medium at 37° C. after 24 h, with the MIC based on three sets of determinations; MIC_{GM} is the geometric mean of the MIC values from seven different strains of Acinetobacter baumanii resistant to Polymyxin B and Colistin, antibiotics of last resort.

TABLE 3
Antimicrobial Activity Against 20 Stains of Acinetobacter baumannii isolated in 2016-2017 resistant to classical antibiotics

| | MIC (μM) | | | | | | | | | | |
|---|---|---|---|---|---|---|---|---|---|---|---|
| | 968886/ | 1001611/ | 1010245/ | 1035794/ | 938370/ | 952659/ | 963659/ | 965463/ | 1017395/ | 1021371/ | MIC_{GM} |
| Peptide^{a} | 977751 | 1035166 | 963618 | 964304 | 981653 | 961997 | 1002956 | 1007660 | 1010282 | 101887 | (μM) |
| With specificity | | | | | | | | | | | |
| determinants | | | | | | | | | | | |
| D84(Lys¹-6 Lys-1) | 0.7 | 0.7 | 0.7 | 0.7 | 0.7 | 0.7 | 0.7 | 0.7 | 0.7 | 0.7 | 0.7 |
| | 0.3 | 0.7 | 0.7 | 1.4 | 0.7 | 1.4 | 0.3 | 0.3 | 0.7 | 0.7 | |
| D86(Lys¹-6 Dab-1) | 1.5 | 0.7 | 0.7 | 1.5 | 0.7 | 1.5 | 0.7 | 3.0 | 3.0 | 1.5 | 1.0 |
| | 0.7 | 0.7 | 0.7 | 1.5 | 0.7 | 0.7 | 0.7 | 0.7 | 0.7 | 0.7 | |
| D105(Lys¹-6 Dap-1) | 1.5 | 0.4 | 0.8 | 0.8 | 0.8 | 1.5 | 1.5 | 1.5 | 1.5 | 1.5 | 1.0 |
| | 0.8 | 0.8 | 0.8 | 1.5 | 0.8 | 1.5 | 0.8 | 0.8 | 0.8 | 0.8 | |
| D101(Lys¹Ser²⁶-5 | 0.7 | 0.7 | 0.7 | 1.4 | 0.7 | 1.4 | 1.4 | 0.7 | 1.4 | 1.4 | 1.0 |
| Lys-1) | 0.7 | 1.4 | 0.7 | 1.4 | 0.7 | 1.4 | 0.7 | 0.7 | 1.4 | 1.4 | |
| D102(Lys¹Ser²⁶-5 | 0.7 | 0.4 | 0.4 | 0.7 | 0.7 | 0.4 | 0.7 | 0.7 | 0.7 | 0.4 | 0.6 |
| Dap-1) | 0.7 | 0.7 | 0.4 | 0.7 | 0.4 | 1.5 | 0.4 | 0.4 | 0.7 | 0.7 | |
| Without specificity | | | | | | | | | | | |
| determinants | | | | | | | | | | | |
| D85(K13A/K16A)- | 3.0 | 1.5 | 1.5 | 3.0 | 3.0 | 1.5 | 0.7 | 3.0 | 3.0 | 1.5 | 1.9 |
| (Lys¹-6 Orn-1) | 6.0 | 1.5 | 1.5 | 1.5 | 1.5 | 1.5 | 1.5 | 1.5 | 1.5 | 3.0 | |
| D86(K13A/K16A)- | 0.8 | 0.8 | 0.8 | 0.8 | 1.5 | 0.8 | 0.4 | 0.8 | 0.8 | 0.8 | 0.9 |
| (Lys¹-6 Dab-1) | 3.1 | 0.8 | 0.8 | 1.5 | 0.4 | 0.8 | 0.8 | 0.8 | 0.8 | 0.5 | |
| D105(K13A/K16A)- | 1.6 | 1.6 | 0.8 | 1.6 | 1.6 | 6.4 | 3.2 | 3.2 | 3.2 | 0.8 | 2.0 |
| (Lys¹-6 Dap-1) | 0.8 | 3.2 | 1.6 | 3.2 | 1.6 | 3.2 | 1.6 | 3.2 | 1.6 | 3.2 | |

### Example 3

### Hemolytic Activity and Therapeutic Indices

The biological activities of the 10 peptide analogs, with and without specificity determinants are shown in Table 4.

TABLE 4
Polar face substitutions of positively charged residues.

| | | | | | HC₅₀ | | | T.I. |
|---|---|---|---|---|---|---|---|---|
| | Net | Peptide | HC₅₀ | HC₅₀ | Fold | MIC_{GM} | | Fold |
| Peptide Name^{a} | Charge | Mass | (μg/mL)^{b} | (μM)^{b} | Improvement^{c} | (μM)^{d} | T.I.^{e} | Improvement^{f} |
| With specificity determinants | | | | | | | | |
| D87(Lys¹-6 Arg-1) | +9 | 3033.7 | 12 | 4.0 | 1.0 | 0.8 | 5.0 | 1.0 |
| D84(Lys¹-6 Lys-1) | +9 | 2865.6 | 155.5 | 54.3 | 13.6 | 0.5 | 108.6 | 21.7 |
| D85(Lys¹-6 Orn-1) | +9 | 2781.5 | 406.5 | 146.1 | 36.5 | 0.5 | 292.2 | 58.4 |
| D86(Lys¹-6 Dab-1) | +9 | 2697.3 | >2000 | >742 | >186 | 1.0 | >742 | >148 |
| D105(Lys¹-6 Dap-1) | +9 | 2613.1 | >3000 | >1148 | >287 | 1.2 | >957 | >191 |
| D101(Lys¹Ser²⁶-5 Lys-1) | +8 | 2824.5 | 279 | 103.9 | 26.0 | 0.8 | 129.9 | 26.0 |
| D102(Lys¹Ser²⁶-5 Dab-1) | +8 | 2684.3 | >2000 | >708 | >177 | 0.7 | >1012 | >202 |
| Without specificity determinants | | | | | Fold Decrease^{c} | | | Fold Decrease^{f} |
| D85(K13A/K16A)-(Lys¹-6 Orn-1) | +7 | 2667.3 | 2.3 | 0.9 | −162.3 | 2.0 | 0.5 | −584.4 |
| D86(K13A/K16A)-(Lys¹-6 Dab-1) | +7 | 2583.1 | 20.0 | 7.7 | >−96.4 | 0.9 | 8.6 | >−86.3 |
| D105(K13A/K16A)-(Lys¹-6 Dap-1) | +7 | 2499.0 | 7.2 | 2.9 | >−395.9 | 1.3 | 2.2 | >−435.0 |

^{a}6 Lys-1 denotes 6 Lys residues on the polar face at positions 3, 7, 11, 18, 22, and 26. 5 Lys-1 denotes 5 Lys residues on polar face at positions 3, 7, 11, 18, and 22 (position 26 is substituted by Ser).
^{b}Hemolytic activity, HC₅₀, is the concentration of peptide that results in 50% hemolysis of human red blood cells after 18 h at 37° C.
^{c}Fold improvement in hemolytic activity, HC₅₀, relative to Arg containing peptide. Fold decrease compares the same peptide with and without specificity determinants. Without specificity determinants shows a dramatic fold decrease in HC₅₀.
^{d}MIC is minimal inhibitory concentration (μM) that inhibited growth of different strains in Mueller-Hinton (MH) medium at 37° C. after 24 hr, with the MIC based on three sets of determinations; MIC_{GM} is the geometric mean of the MIC values from seven different strains of Acinetobacter baumanii resistant to Polymyxin 8 and Colistin, antibiotics of last resort.
^{e}Therapeutic index (T.I.) was calculated from HC₅₀ (μM)/MIC_{GM} (μM). Fold decrease compares the same peptide with and without specificity determinants. Without specificity determinants shows a dramatic fold decrease in the therapeutic index.
^{f}Fold improvement in therapeutic index, relative to the Arg containing peptide.

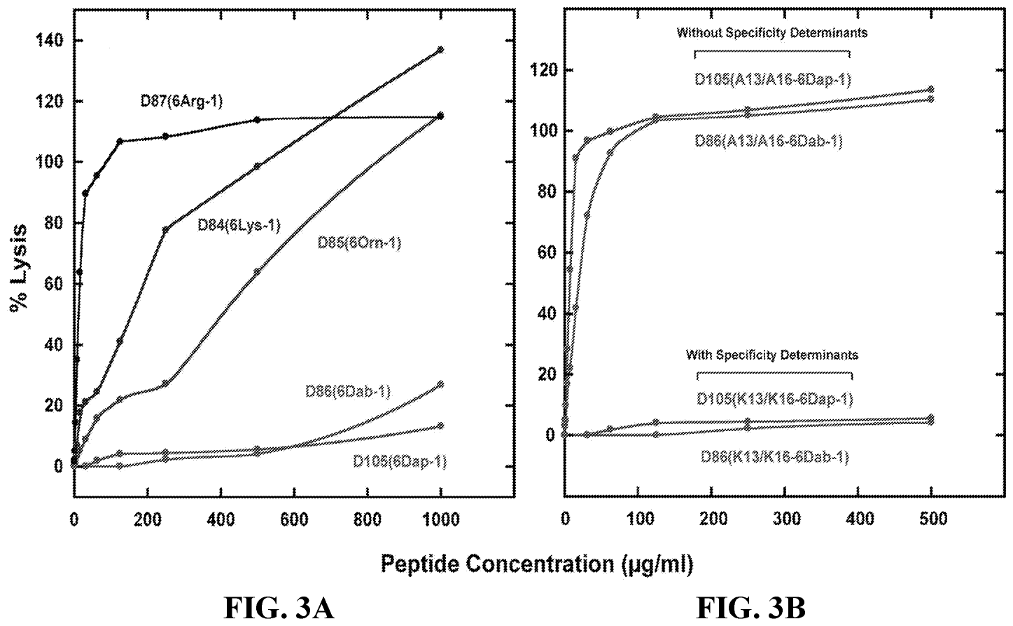

As shown in Table 4, the three peptides without specificity determinants are extremely hemolytic, with HC₅₀ values (the peptide concentration required for 50% hemolysis) of 0.9 μM to 7.7 μM, which is of comparable magnitude to the antimicrobial activity of 0.9 to 2.0 μM. Thus, the therapeutic indices vary from 0.5 to 8.6 depending on the positively-charged residue used on the polar face (Table 4). The specificity determinants have very little effect on antimicrobial activity where the geometric mean MIC ranges from 0.5 μM to 1.2 μM for the seven AMPs compared to 0.9 μM to 2.0 μM without specificity determinants. The specificity determinants result in dramatic decreases in hemolytic activities from a range of 0.9 to 7.7 μM for the HC₅₀ for AMPs lacking specificity determinants to 4.0 to >1148 μM depending on the positively charged residue on the polar face of the AMP. This corresponds to increases in the therapeutic indices from 5.0 to >1012 depending on the AMP. Our best AMP shows an increase in the therapeutic index of >202-fold relative to the Arg -containing peptide (Table 4). Our results show that the improvements in the hemolytic activity, and thus the therapeutic indices, depends on the type of positively-charged residue used on the polar face. The HC₅₀ value for Arg in the six polar face positions (3, 7, 11, 18, 22, and 26) is 4.0 μM compared to Lys (54.3 μM) and Orn (146.1 μM). Thus, the use of Orn residues instead of Arg provides a 37-fold decrease in the hemolytic activity or a 58-fold improvement in the therapeutic index. The dramatic and unexpected decrease in hemolytic activity resulted from the use of the two unusual amino acid residues Dab and Dap on the polar face. There was >186-fold decrease in hemolytic activity relative to Arg-containing AMP when using diaminobutyric acid (4 μM to >742 μM) and >287-fold decrease in hemolytic activity when using diaminopropionic acid (4 μM to >1148 μM). Because the antimicrobial activity does not vary significantly between peptides, the changes in therapeutic indices show a similar large increase in fold improvement relative to Arg when the amino acid residues are changed systematically from Arg to Lys, Orn, Dab and Dap. We are systematically decreasing the number of carbon atoms in the side-chain from four in the case of lysine to one in the case of a diaminopropionic acid residue. This results in a change in hemolytic activity from 54.3 μM for Lys to >1148 μM for Dap, a greater than 21-fold change in hemolytic activity resulting in a therapeutic index of 108.6 for Lys and greater than 957 for Dap (Table 4). The significance of this change from Arg, Lys, Orn to Dab or Dap is observed graphically in a plot of percent lysis of human red blood cells versus peptide concentration up to 1000 μg/ml (FIG. 3A). No significant lysis is observed for Dab- or Dap-containing peptides. The importance of using these unusual amino acids in place of Lys or Arg residues in antimicrobial peptides on the polar face cannot be over emphasized. Removing the C-terminal positively-charged residue has no effect on the hemolytic activity or therapeutic index (compare peptide D101(5 Lys-1) to D84(6 Lys-1) or D102(5 Dab-1) to D86(6 Dab-1) (Table 4). The importance of specificity determinants is shown in FIG. 3B where the six Dab- and six Dap-containing peptides without specificity determinants (Ala13/Ala16) are extremely hemolytic compared to the same peptides with specificity determinants (Lys13/Lys16) which show no lysis of human red blood cells. This is an unprecedented and completely unexpected result.

### Example 4

### Antimicrobial Activity of AMPs in the Presence of Human Sera

Because only unbound AMP is available to interact with the therapeutic target, the extent of binding between AMPs and serum proteins can have significant effects on efficacy of these drugs. To address this issue, we determined the MIC values of our peptides in the presence of Mueller Hinton (MH) medium and MH medium supplemented with 25% (v/v) human sera. This assay estimates the in vivo bioavailability of our AMPs. The appropriate non-specific affinity of an AMP for serum proteins can significantly improve in vivo half-life and decrease clearance. An increase in MIC in the presence of serum is attributed to inhibition of antimicrobial activity due to binding to serum proteins.

TABLE 5
Antimicrobial activity against A. baumannii strain
649 in the presence and absence of 25% human sera

| | MIC(μM) | |
|---|---|---|
| Peptide Name | No serum | 25% human serum |

With specificity determinants (Lys¹³Lys¹⁶)

| | | |
|---|---|---|
| D87(Lys¹-6 Arg-1) | 0.7 | 1.3 |
| D84(Lys¹-6 Lys-1) | 0.3 | 0.3 |
| D85(Lys¹-6 Orn-1) | 0.4 | 0.2 |
| D86(Lys¹-6 Dab-1) | 0.7 | 0.2 |
| D105(Lys¹-6 Dap-1) | 0.8 | 3.0 |
| D101(Lys¹Ser²⁶-5 Lys-1) | 0.7 | 0.7 |
| D102(Lys¹Ser²⁶-5 Dab-1) | 0.4 | 0.7 |

Without specificity determinants (Ala¹³Ala¹⁶)

| | | |
|---|---|---|
| D85(K13A/K16A)-(Lys¹-6 Orn-1) | 1.5 | Precipitate |
| D86(K13A/K16A)-(Lys¹-6 Dab-1) | 0.8 | 3.0 |
| D105(K13A/K16A)-(Lys¹-6 Dap-1) | 0.8 | 12.5 |

As shown in Table 5, the three AMPs without specificity determinants have reduced antimicrobial activity against A. baumannii strain 649 (a blood isolate) in the presence of 25% human sera. In contrast, for the seven AMPs with specificity determinants, six have excellent activity against A. baumannii, blood strain 649 in the presence of 25% human sera except for peptide D105 which shows a 4-fold decrease in MIC. Peptide D85, without specificity determinants, caused precipitation when peptides were added to the assay mixture containing serum (after 18 h incubation, more precipitation was observed), while D86 and D105, without specificity determinants resulted in a 4-fold and 15-fold loss of antimicrobial activity, respectively (Table 5). These results emphasize the importance of specificity determinants in preventing any significant loss of antimicrobial activity. Thus, specificity determinants have three major roles: maintaining or enhancing antimicrobial activity, preventing binding to serum proteins, and decreasing α-helical structure in aqueous conditions, but allowing inducible helical structure within the hydrophobicity of the membrane.

### Example 5

### Peptide Hydrophobicity

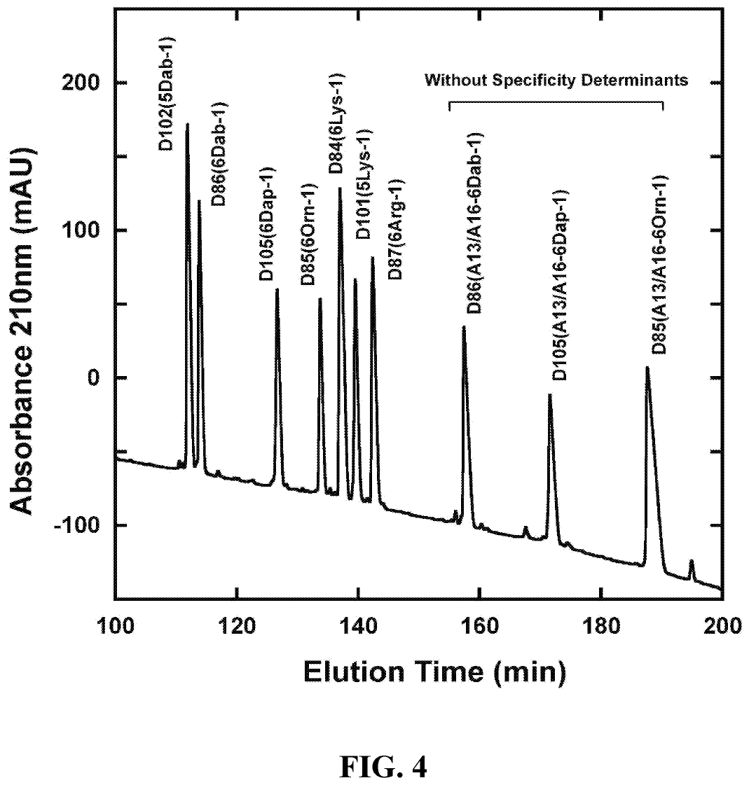

Retention behavior in reversed-phase chromatography is an excellent method to represent overall peptide hydrophobicity. Retention times of amphipathic α-helical peptides are highly sensitive to the conformational status of the peptides upon interaction with the hydrophobic surface of the column matrix. The non-polar face of amphipathic α-helical peptides represents the preferred binding domain for interaction with the hydrophobic matrix of the reversed-phase column. In this study, the observed peptide retention times are relative hydrophobicities because they are dependent on the TFA concentration and organic solvent in the mobile phase, gradient rate, column temperature, flow-rate and column used. The three AMPs without specificity determinants have hydrophobic residues on the non-polar face of the helix (8 Leu residues in two clusters (L2, L4, L6 and L9 in the N-terminal cluster and L17, L20, L21 and L24 in the C-terminal cluster and 2 Ala residues at positions 13 and 16 between the two clusters of Leu residues (FIG. 2). This hydrophobic surface is the preferred binding domain for binding to the hydrophobic surface on the column matrix, however, the overall hydrophobicity is also affected by the composition of residues on the polar face which contains six positively-charged residues (FIG. 2). The amino acid composition on the polar face has the positively-charged residues in the same positions (3, 7, 11, 18, 22 and 26) but varies the type of positively-charged residue from either six Arg, Lys, Orn, Dab or Dap residues. The seven AMPs with specificity determinants have two Lys residues between the two hydrophobic clusters (FIG. 2), decreasing the overall hydrophobicity. Thus, the overall hydrophobicity of the five AMP with +9 charge varied from 115.8 to 143.2 min, considerably less than the peptides without specificity determinants which varied from 158.3 to 188.7 min (FIG. 4 and Table 6). All ten peptides used in this study could be readily separated by reversed-phase chromatography with the seven peptides with specificity determinants being much more hydrophilic than the three control peptides without specificity determinants (FIG. 4). The type of positively-charged residue on the polar face has a dramatic effect on the overall hydrophobicity with the Dab residue being more hydrophilic (less hydrophobic) than the Dap residue even though the Dab residues are a carbon atom larger in their side-chain compared to the Dap residues (Dab peptide (D86) retention time 115.8 min compared to Dap peptide (D105) retention time of 127.9 min). This can be explained by the Dab residues stabilizing the α-helical structure considerably more than Dap residues. This means that the polar face of Dab residues is interacting more with the hydrophobic matrix than the polar face of Dap residues, which results in the large decrease in retention time (t_{R} for Dap is 127.9 min and t_{R} for Dab is 115.8 min, i.e., a decrease of 12.1 min) even though each Dab residue has one more carbon atom in its side chain than the Dap residue (Table 6).

All our AMPs shown in Table 1 have the identical hydrophobic density with eight Leu residues on the non-polar face. The hydrophobic density of our de novo designed AMPs is similar to that observed for native AMPs of 22-27 residues (see review by Hodges et al, 2012 In, Development of Therapeutic Agents Handbook; Wiley and Sons Inc. 2012, pp. 285-358). Hydrophobic density is calculated by the sum of the hydrophobicity values of non-polar residues (Pro, Val, Ile, Leu, Met, Tyr, Phe, and Trp) in the AMP divided by the number of residues in the peptide.

### Example 6

### Peptide Secondary Structure and Amphipathicity

Table 6 shows the circular dichroism results for the 10 peptides used in this study in conditions of pH 7 (50 mM PO₄, 100 mM KCl) and in the presence of 50% trifluoroethanol (TFE), a mimic of the hydrophobicity and the α-helix inducing ability of the hydrophobic membrane. The two Lys specificity determinants substituted in the center of the non-polar face was to disrupt the continuous hydrophobic surface on the non-polar face. A continuous hydrophobic surface stabilizes α-helical structure. Our design concept was to minimize α-helical structure in aqueous conditions and maximize the inducible α-helical structure in the presence of the hydrophobicity of the membrane.

TABLE 6
Biophysical data.

| | | Hydropho- | | | | | | | | |
|---|---|---|---|---|---|---|---|---|---|---|
| | | bicity^{b} | | | | Δ[Θ]₂₂₂ | % | | | Amphi- |
| | Net | pH 2 | Aqueous pH 7 | | 50% TFE | TFE- | Helix^{d} | Tp ^{e} | P_{A} ^{f} | pathicity ^{g} |
| Peptide Name^{a} | charge | t_{R} | [θ]₂₂₂ ^{c} | % Helix^{d} | [θ]₂₂₂ ^{c} | aqueous | Induced | (° C.) | (min) | pH 2 |

With specificity determinants

| | | | | | | | | | | |
|---|---|---|---|---|---|---|---|---|---|---|
| D87(Lys¹-6 Arg-1) | +9 | 143.2 | 10,192 | 33 | 31,308 | 21,116 | 67 | 21.2 | 5.9 | 3.625 |
| D84(Lys¹-6 Lys-1) | +9 | 138.1 | 8,230 | 24 | 33,653 | 25,423 | 76 | 21.2 | 5.9 | 3.327 |
| D85(Lys¹-6 Orn-1) | +9 | 134.7 | 4,808 | 13 | 38,423 | 33,615 | 87 | 17.3 | 4.1 | 3.420 |
| D86(Lys¹-6 Dab-1) | +9 | 115.8 | 1,769 | 6 | 27,923 | 26,154 | 94 | 5.0 | 0 | 3.879 |
| D105(Lys¹-6 Dap-1) | +9 | 127.9 | 5,961 | 25 | 24,192 | 18,231 | 75 | 16.9 | 3.7 | 3.570 |
| D101(Lys¹Ser²⁶-5 | +8 | 140.3 | 2,538 | 7 | 38,538 | 36,000 | 93 | 21.2 | 6.0 | 3.419 |
| Lys-1) | | | | | | | | | | |
| D102(Lys¹Ser²⁶-5 | +8 | 113.9 | 5,962 | 25 | 23,385 | 17,423 | 75 | 5.0 | 0 | 3.842 |
| Dab-1) | | | | | | | | | | |

Without specificity determinants

| | | | | | | | | | | |
|---|---|---|---|---|---|---|---|---|---|---|
| D85(K13A/K16A)- | +7 | 188.7 | 9,269 | 51 | 18,038 | 8,769 | 49 | 41.0 | 28.0 | 4.631 |
| (Lys¹-6 Orn-1) | | | | | | | | | | |
| D86(K13A/K16A)- | +7 | 158.3 | 2,308 | 12 | 18,731 | 16,423 | 88 | 31.0 | 12.5 | 5.135 |
| (Lys¹-6 Dab-1) | | | | | | | | | | |
| D105(K13A/K16A)- | +7 | 172.3 | 9,577 | 53 | 18,038 | 8,461 | 47 | 39.0 | 18.0 | 4.797 |
| (Lys¹-6 Dap-1) | | | | | | | | | | |

^{a}The D denotes that all amino acids in each peptide are in the D-conformation except for Dab and Dap residues which are in the L-conformation.
^{b}t_{R} denotes retention time in RP-HPLC at pH 2 at a temperature of 25° C., and is a measure of overall peptide hydrophobicity.
^{c} The mean residue molar ellipticities [θ]₂₂₂(mdeg cm²/(dmol*res)) at a wavelength of 222 nm were measured at 25° C. in aqueous conditions (100 mM KCl, 50 mM Na₂HPO₄/NaH₂PO₄, pH 7.0) or in aqueous buffer containing 50% trifluoroethanol (TFE) by circular dichroism spectroscopy.
^{d} The helical content (as a percentage) of a peptide is relative to the molar ellipticity value of the peptide in the presence of 50% TFE. % helix induced is the increase in molar ellipticity (as a percentage) of the peptide in the presence of 50% TFE.
^{e} Tp, temperature at which maximum retention time is observed over the temperature range 5-77° C.
^{f} P_{A} denotes the sell-association parameter (dimerization/oligomerization) of each peptide during RP-HPLC temperature profiling, which is the maximal retention time difference of (t_{R}¹-t_{R}⁵ for peptide analogs) − (t_{R}¹-t_{R}⁵ for control peptide RC) within the temperature range; t_{R}¹-t_{R}⁵ is the retention time difference of a peptide at a specific temperature (t_{R}¹) compared with that at 5° C. (t_{R}⁵). The sequence of the random coil control peptide (RC) is Ac-ELEKGGLEGEKGGKELEK-amide.
^{g} Amphipathicity was determined by calculation of the hydrophobic moment [Eisenberg et. al, 1982] using hydrophobicity coefficients determined by RP-HPLC at pH 2.

The % helix induced in 50% TFE varied from 67% to 94% for the AMPs with specificity determinants depending on the type of positively charged residue used on the polar face (Table 6). It is interesting that when using six diaminobutyric acid residues on the polar face in aqueous conditions, the peptide had the least α-helical structure (6%) and the highest inducible α-helical structure in the presence of 50% TFE (94%). For peptides with specificity determinants, the amphipathicity ranged from a low of 3.327 to 3.879 depending on the positively charged residue used on the polar face. As expected, removing the Lys specificity determinants at positions 13 and 16 and replacing them with Ala residues increased amphipathicity which ranged from 4.631 to 5.135 depending on the positively charged residue used on the polar face. All the +9 peptides shown in Table 1G are identical in sequence except for the six polar face substitutions which were either Arg, Lys, Orn, Dab or Dap residues. Thus, amphipathicity is affected by the hydrophobicity coefficient used for the calculation of amphipathicity. These values were: Arg (0.6), Lys (2.8), Orn (2.1),

Dab (−1.2) and Dap (1.0). Thus, the peptide with the highest amphipathicity was the Dab-containing peptide 3.879 (with specificity determinants) and 5.135 without specificity determinants (Table 6). The amphipathicity values of our AMPs are similar to those observed for native AMPs in the length of 22-27 residues (see review by Hodges et al, 2012 In, Development of Therapeutic Agents Handbook; Wiley and Sons Inc. 2012, pp. 285-358). In summary, both amphipathicity and inducible α-helical structure play a critical role in providing AMPs with the desired properties.

### Example 7

### Peptide Self-Association

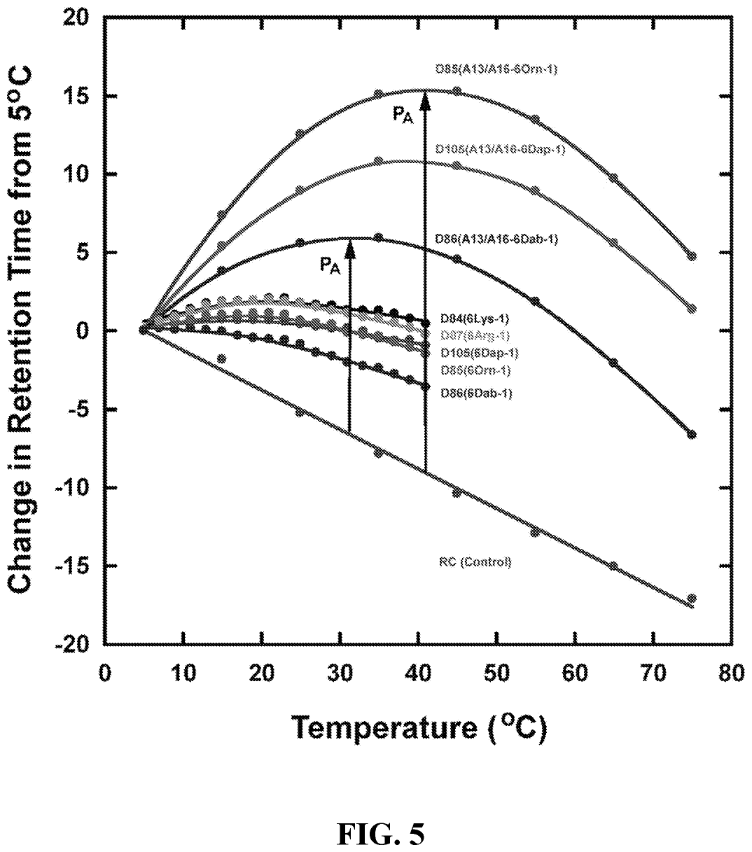

Peptide self-association, the ability to dimerize/oligomerize in aqueous solution, is a very important parameter to optimize antimicrobial activity and toxicity as measured by hemolytic activity. We have proposed that the monomeric random-coil antimicrobial peptides in aqueous solution are best suited to pass through a polysaccharide capsule, the outer membrane lipopolysaccharide and the cell wall peptidoglycan layer of Gram-negative bacteria prior to penetration into the cytoplasmic membrane, where there is induction of α-helical structure and disruption of membrane structure, cell leakage and death of bacterial cells. In contrast, if the self-association ability of an AMP in aqueous medium is too strong, stable folded dimers/oligomers are formed through interaction of their non-polar faces which decreases the ability of the AMP to dissociate to monomer and the dimer/oligomer to effectively pass through the capsule and cell wall to reach the cytoplasmic membrane. The ability of an AMP to self-associate was determined by a RP-HPLC technique developed in our laboratory, referred to as temperature profiling over the temperature range 5° C. to 80° C. FIG. 5 shows the retention behavior of three AMPs without specificity determinants (D85(K13A/K16A-6 Orn-1); D105(K13A/K16A-6 Dap-1) and D86(K13A/K16A-6 Dab-1) (top of FIG. 5), and five AMPs with specificity determinants (D87-6 Arg-1; D84-6 Lys-1; D86-6 Orn-1); D86-6 Dab-1 and D105-6 Dap-1) over the temperature range 5-77° C. These eight AMPs are compared to a random-coil control peptide denoted RC. RC is an 18-residue monomeric random-coil peptide in both aqueous and hydrophobic media and shows a linear decrease in retention time with increasing temperature and is representative of peptides which have no ability to self-associate during reversed-phase chromatography. This linear decrease in retention behavior with increasing temperature represents the general effects of temperature due to greater solute diffusivity and enhanced mass transfer between the mobile and stationary phases. The difference in retention time between an amphipathic α-helical antimicrobial peptide and the RC control peptide is a measure of the ability of a peptide to associate. The association parameter, P_{A} is large for AMPs without specificity determinants (Ala 13 and Ala 16) ranging from 12.5 to 28.0 min (Table 6) and is shown by a black arrow in FIG. 5. The association parameter, P_{A}, is dramatically smaller for AMPs with specificity determinants (Lys 13 and Lys 16 in the center of the non-polar face) and range from 0 to 6 min (Table 6). Thus, specificity determinants dramatically lower self-association, which is a desired property of effective AMPs. Effective AMPs have low self-association in aqueous medium to prevent dimerization and thus can more easily pass through the capsule and cell wall as a random coil monomer to reach the cytoplasmic membrane where the AMPs must be able to be induced into α-helical structure by the hydrophobicity of the membrane and disrupt the membrane causing leakage and death of the bacterial cell. We have shown previously that AMPs which strongly self-associate by their hydrophobic face (i.e., that are too hydrophobic on their non-polar face) show weak antimicrobial activity (Chen et al., J. Biol. Chem. 2005, 13:12316-29; Chen et al., Antimicrob. Agents Chemother. 2007, 51:1398-1406).

### Example 8

### Effect of Location and Type of Positively-Charged Residues on the Polar Face

Two series of five AMPs were synthesized and tested to study the effect of the location and type of positively-charged residues on the polar face of AMPs of this disclosure. The sequences of the AMPs are shown in Table 7. Each AMP has 5 or 6 positively-charged residues on the polar face, in addition to the positively-charged D-Lys amino acids at positions 13 and 16 (i.e., the specificity determinants).

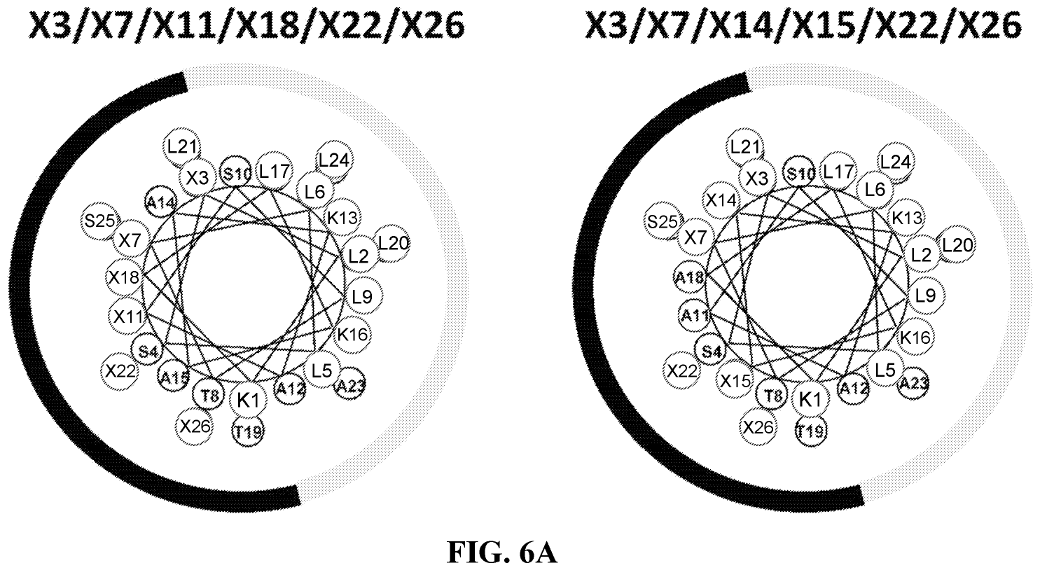

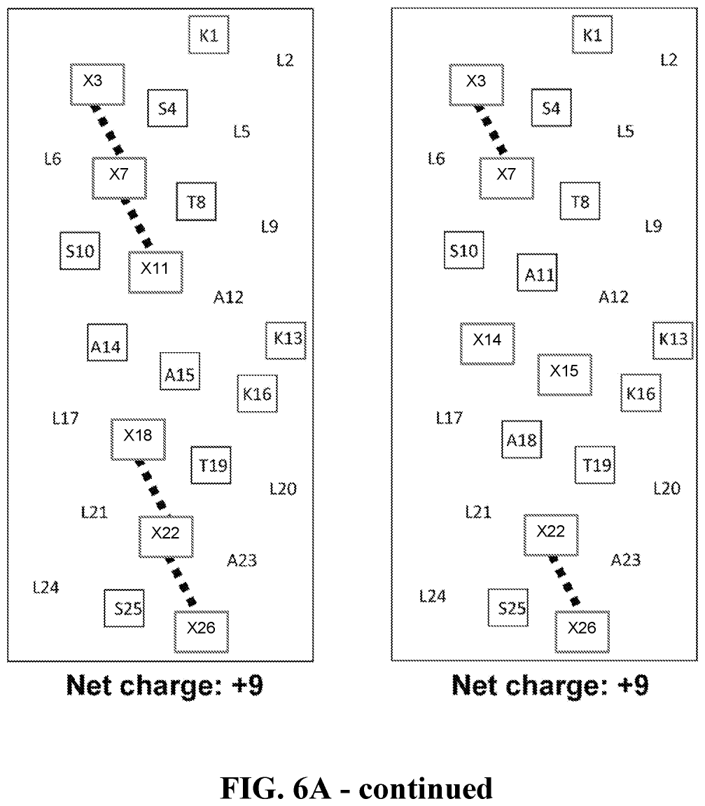

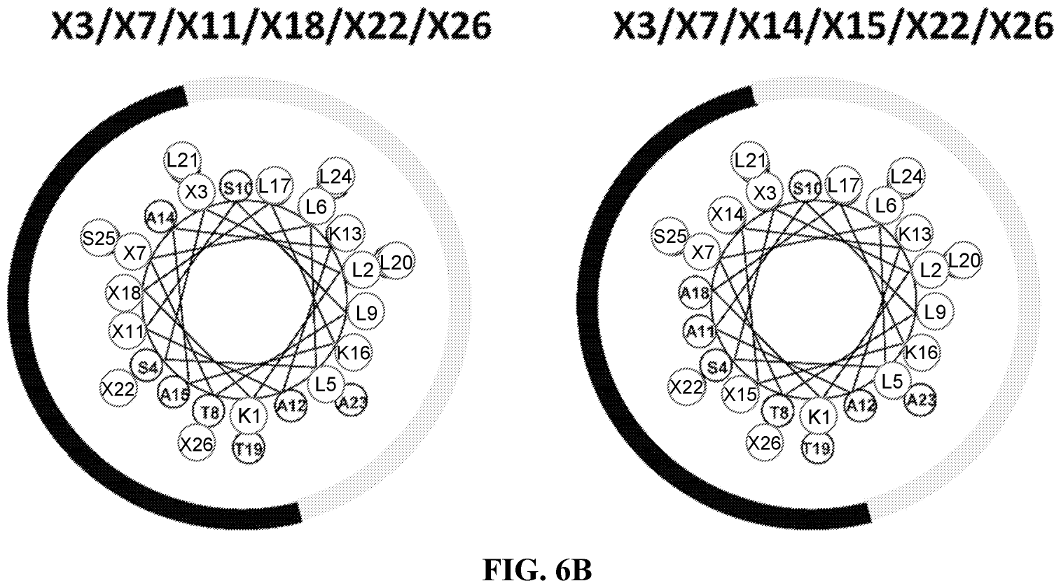

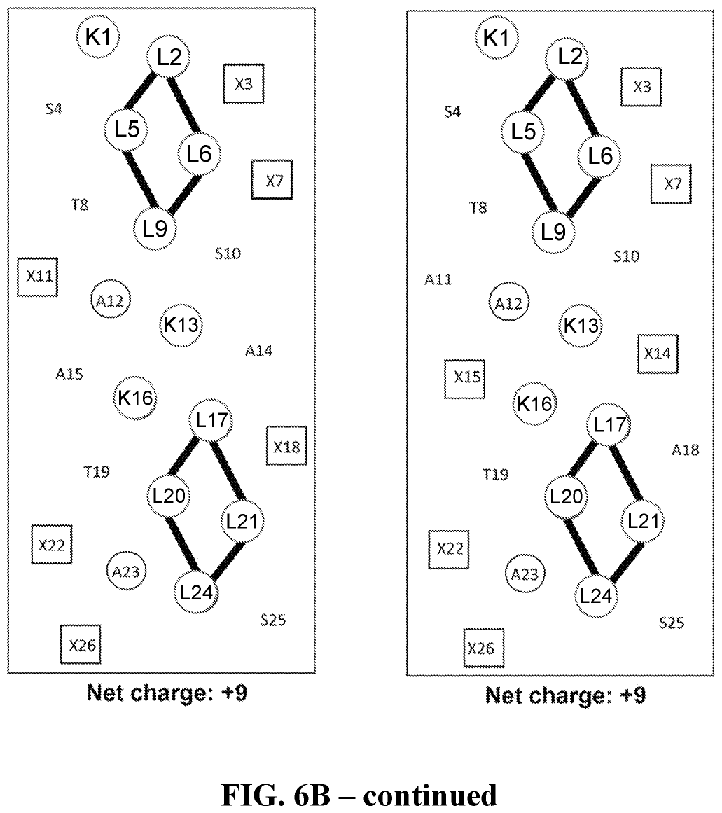

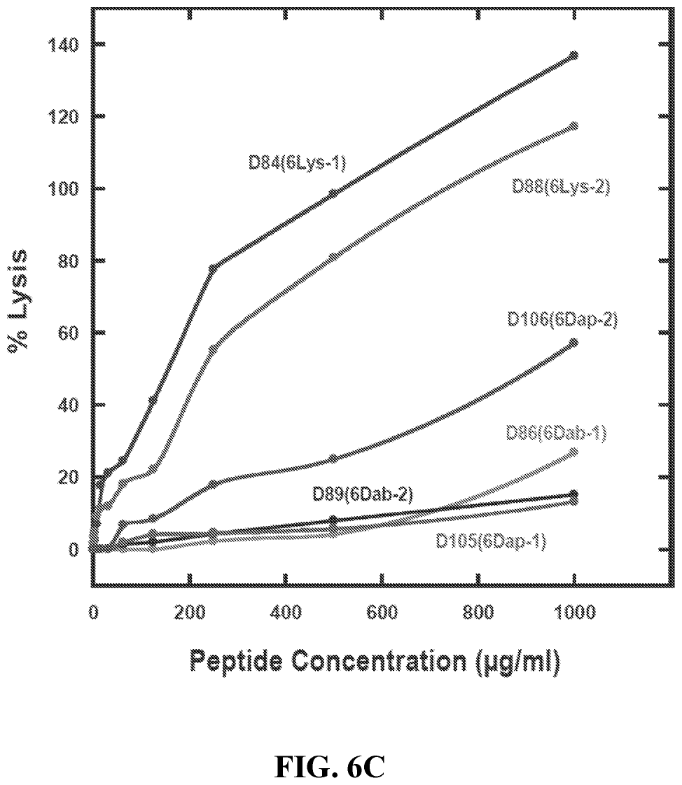

The additional positively-charged residues included D-Lys or L-Dab (2,4-diaminobutyric acid) or L-Dap (2,3-diaminopropionic acid) at positions 3, 7, 11, 18, 22, and 26 (series 1; the first five AMPs of Table 7; FIG. 6A; biophysical data shown in Table 9) or positions 3, 7, 14, 15, 22, and 26 (series 2; the second five AMPs of Table 7; FIG. 6B) of these 26-residue AMPs. Thus, all the amino acids in these ten AMPs were in the D-conformation with the exception of the positively-charged L-Dab or L-Dap residues, which are in the L-confirmation, as shown in Table 7.

Hemolytic activity against human red blood cells and antimicrobial activity against seven Acinetobacter baumannii strains resistant to polymyxin B and colistin were determined using the methodology described above. As shown in Table 8, changing the locations of L-Dab and L-Dap residues on the polar face of the AMPs results in a change in hemolytic activity from 54.3 μM for Lys to >1490 μM for L-Dab, a greater than 27-fold change in hemolytic activity resulting in a therapeutic index of 108.6 for Lys and greater than 1860 for L-Dab. The significance of this change from D-Lys, to L-Dab or L-Dap is observed graphically in a plot of percent lysis of human red blood cells versus peptide concentration up to 1000 μg/ml (FIG. 6C). Thus, changing the type of positively-charged residue from D-Lys, to L-Dab or L-Dap as well as the locations of L-Dab and L-Dap residues on the polar face of these AMPs of this disclosure plays a very important role in obtaining the best therapeutic indices for these AMPs. These significant positive gains in therapeutic index for these AMP resulting from the type and location of positively-charged residues on the polar face was a completely unexpected result that was not predicted by any of the inventors' previous research.

TABLE 7
Polar face substitutions of positively charged residues in AMPs

| Peptide | Net | Sequence^{b} |
|---|---|---|
| Name^{a} | Charge | Specificity determinants (Lys13/Lys16) on non-polar face |
| Amino acid position | | 1 3 7 11 18 22 26 |
| D84(Lys¹-6Lys-1) | +9 | Ac-KL(Lys)SLL(Lys)TLS(Lys)AKAAKL(Lys)TLL(Lys)ALS(Lys)-amide |
| D86(Lys¹-6Dab-1) | +9 | Ac-KL(L-Dab)SLL(L-Dab)TLS(L-Dab)AKAAKL(L-Dab)TLL(L-Dab)ALS(L-Dab)-amide |
| D105(Lys¹-6Dap-1) | +9 | Ac-KL(L-Dap)SLL(L-Dap)TLS(L-Dap)AKAAKL(L-Dap)TLL(L-Dap)ALS(L-Dap)-amide |
| D101(Lys¹Ser²⁶-5Lys-1) | +8 | Ac-KL(Lys)SLL(Lys)TLS(Lys)AKAAKL(Lys)TLL(Lys)ALS(Ser)-amide |
| D102(Lys¹Ser²⁶-5Dab-1) | +8 | Ac-KL(L-Dab)SLL(L-Dab)TLS(L-Dab)AKAAKL(L-Dab)TLL(L-Dab)ALS(Ser)-amide |
| Amino acid position | | 1 3 7 14 15 22 26 |
| D88(Lys¹-6Lys-2) | +9 | Ac-KL(Lys)SLL(Lys)TLSAAK(Lys)(Lys)KLATLL(Lys)ALS(Lys)-amide |
| D89(Lys¹-6Dab-2) | +9 | Ac-KL(L-Dab)SLL(L-Dab)TLSAAK(L-Dab)(L-Dab)KLATLL(L-Dab)ALS(L-Dab)-amide |
| D106(Lys¹-6Dap-2) | +9 | Ac-KL(L-Dap)SLL(L-Dap)TLSAAK(L-Dap)(L-Dap)KLATLL(L-Dap)ALS(L-Dap)-amide |
| D103(Lys¹Ser²⁶-5Lys-2) | +8 | Ac-KL(Lys)SLL(Lys)TLSAAK(Lys)(Lys)KLATLL(Lys)ALS(Ser)-amide |

| | | |
|---|---|---|
| D104(Lys¹Ser²⁶-5Dab-2) | +8 | Ac-KL(L-Dab)SLL(L-Dab)TLSAAK(L-Dab)(L-Dab)KLATLL(L-Dab)ALS(Ser)-amide |

^{a}The ‘D’ denotes all amino acid residues in each peptide are in the D-conformation except for L-Dab and L-Dap residues, which are in the L-conformation. Specificity determinants are positively charged residues in the center of the non-polar face (Lys13/Lys16) (FIG. 2).
^{b}Peptide sequences are shown using the one-letter code for all amino acid residues except at X¹, X², X³, X⁴, X⁵ and X⁶, where the three-letter code is used. Ac denotes N^{α}-acetyl and amide denotes C^{α}-amide. Positions X¹, X², X³, X⁴, X⁵ and X⁶ are positively charged residues (Lys, L-Dab and L-Dap) on the polar face of the amphipathic α-helix (FIG. 1); −1 denotes 6 positively charged residues on the polar face at positions 3, 7, 11, 18, 22 and 26 or 5 positively charged residues on the polar face at positions 3, 7, 11, 18 and 22 (position 26 is substituted by Ser); -2 denotes 6 positively charged residues on the polar face at positions 3, 7, 14, 15, 22 and 26 or 5 positively charged residues on the polar face at positions 3, 7, 14, 15 and 22 (position 26 is substituted by Ser).

TABLE 8
Antibacterial activity against 7 strains of Acinetobacter baumannii resistant to Polymyxin B and Colistin, hemolytic activity and therapeutic index

| | | MIC | | | | | | | | | |
|---|---|---|---|---|---|---|---|---|---|---|---|
| | Peptide | (μm)^{b} | | | | | | | MIC_{GM} | HC₅₀ | |
| Peptide Name^{a} | Mass | MB9941 | MB9949 | MB9951 | MB9952 | MB9953 | MB9955 | MB9963 | (μM)^{b} | (μM)^{c} | T.I.^{d} |
| D84(Lys¹-6 Lys-1) | 2865.6 | 0.3 | 0.7 | 0.3 | 0.7 | 0.7 | 0.3 | 0.7 | 0.5 | 54.3 | 108.6 |
| D86(Lys¹-6 Dab-1) | 2697.3 | 1.5 | 0.8 | 0.8 | 0.8 | 0.8 | 0.8 | 0.8 | 0.9 | >742 | >824 |
| D105(Lys¹-6 Dap-1) | 2613.1 | 0.8 | 0.8 | 3.0 | 0.8 | 3.0 | 0.5 | 1.5 | 1.2 | >1148 | >957 |
| D101(Lys¹Ser²⁶-5 Lys-1) | 2824.5 | 0.7 | 0.7 | 0.7 | 0.7 | 1.5 | 0.7 | 0.7 | 0.8 | 103.9 | 129.9 |
| D102(Lys¹Ser²⁶-5 Dab-1) | 2684.3 | 0.4 | 0.7 | 0.7 | 0.7 | 1.4 | 0.7 | 0.7 | 0.7 | >708 | >1012 |
| D88(Lys¹-6 Lys-2) | 2865.6 | 0.7 | 0.7 | 0.3 | 0.3 | 0.3 | 0.3 | 0.4 | 0.4 | 80.6 | 201.5 |
| D89(Lys¹-6 Dab-2) | 2697.3 | 0.7 | 0.7 | 0.7 | 0.7 | 0.7 | 0.7 | 0.7 | 0.7 | >1112 | >1589 |
| D106(Lys¹-6 Dap-2) | 2613.1 | 0.8 | 0.8 | 1.5 | 0.8 | 0.8 | 0.4 | 0.8 | 0.8 | 340.2 | 425.3 |
| D103(Lys¹Ser²⁶-5 Lys-2) | 2824.5 | 0.7 | 0.7 | 0.7 | 0.7 | 0.7 | 0.7 | 0.7 | 0.7 | 134.9 | 192.7 |
| D104(Lys¹Ser²⁶-5 Dab-2) | 2684.3 | 0.7 | 0.7 | 0.7 | 0.7 | 1.5 | 0.7 | 0.7 | 0.8 | >1490 | >1863 |
| Colistin | 1155.5 | >28 | >28 | >28 | >28 | >28 | >28 | >28 | >28 | | |
| Polymyxin B | 1301.6 | >25 | >25 | >25 | >25 | >25 | >25 | >25 | >25 | | |

^{a}The sequences and the -1 or -2 designations are described in Table 7.
^{b}MIC is minimal inhibitory concentration (μM) that inhibited growth of different strains in Mueller-Hinton (MH) medium at 37° C. after 24 h, with the MIC based on three sets of determinations; MIC_{GM} is the geometric mean of the MIC values from seven different strains of Acinetobacter baumanii resistant to Polymyxin B and Colistin, antibiotics of last resort. Colistin and Polymyxin B results provided by MERCK.
^{c}Hemolytic activity, HC₅₀, is the concentration of peptide that results in 50% hemolysis of human red blood cells after 18 h at 37° C.
^{d}Therapeutic index (T.I.) was calculated from HC₅₀ (μM)/MIC_{GM} (μM).

TABLE 9
Biophysical data

| | | Hydropho- | | | | | % |
|---|---|---|---|---|---|---|---|
| | Net | bicity^{b} | Aqueous pH 7 | | 50% TFE | Δ[Θ]₂₂₂ | Helix^{d} |
| Peptide Name^{a} | charge | pH 2 t_{R} | [θ]₂₂₂ ^{c} | % Helix^{d} | [θ]₂₂₂ ^{c} | TFE-aqueous | Induced |

With specificity determinants

| | | | | | | | |
|---|---|---|---|---|---|---|---|
| D84(Lys¹-6 Lys-1) | +9 | 138.1 | 8,230 | 24 | 33,653 | 25,423 | 76 |
| D86(Lys¹-6 Dab-1) | +9 | 115.8 | 1,769 | 6 | 27,923 | 26,154 | 94 |
| D105(Lys¹-6 Dap-1) | +9 | 127.9 | 5,961 | 25 | 24,192 | 18,231 | 75 |
| D101(Lys¹Ser²⁶-5 Lys-1) | +8 | 140.3 | 2,538 | 7 | 38,538 | 36,000 | 93 |
| D102(Lys¹Ser2⁶-5 Dab-1) | +8 | 113.9 | 5,962 | 25 | 23,385 | 17,423 | 75 |

^{a}The D denotes that all amino acids in each peptide are in the D-conformation.
^{b}t_{R} denotes retention time in RP-HPLC at pH 2 at a temperature of 25° C. and is a measure of overall peptide hydrophobicity.
^{c} The mean residue molar ellipticities [θ]₂₂₂ (mdeg cm²/(dmol*res)) at a wavelength of 222 nm were measured at 25° C. in aqueous conditions (100 mM KCl, 50 mM Na₂HPO₄/NaH₂PO₄, pH 7.0) or in aqueous buffer containing 50% trifluoroethanol (TFE) by circular dichroism spectroscopy.
^{d}The helical content (as a percentage) of a peptide is relative to the molar ellipticity value of the peptide in the presence of 50% TFE. % helix induced is the increase in molar ellipticity (as a percentage) of the peptide in the presence of 50% TFE.

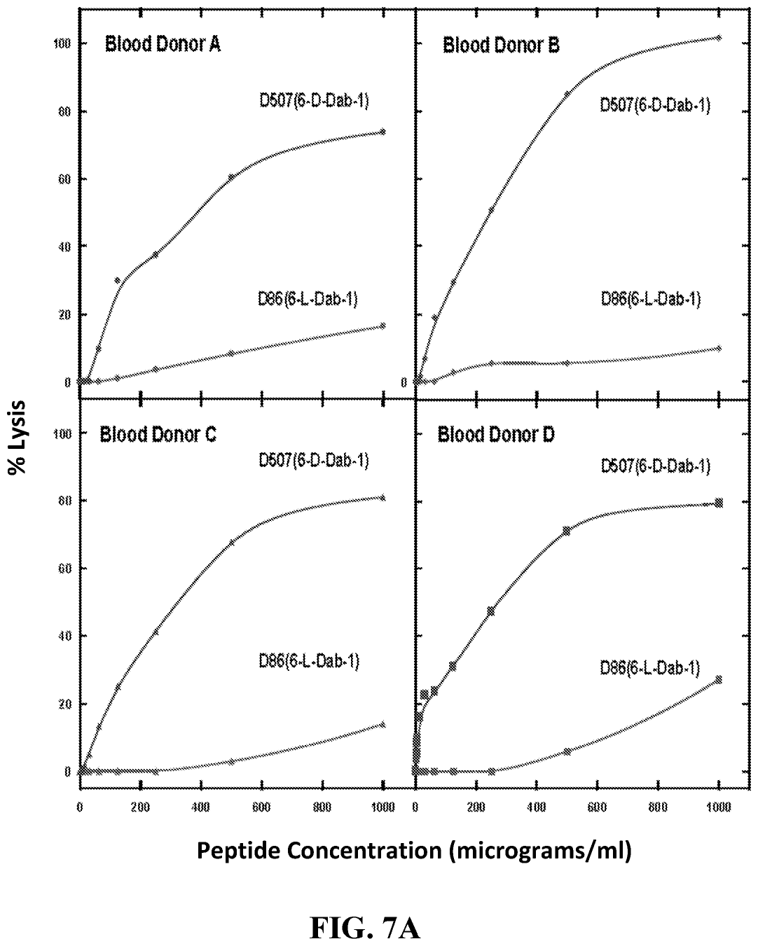

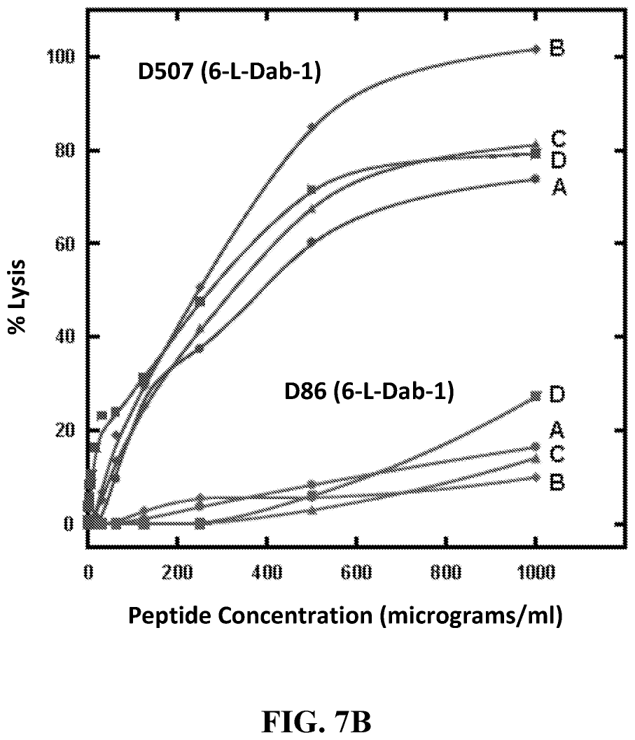

Hemolytic activity against human red blood cells collected from four separate human blood donors was tested for two antimicrobial peptides (D507(6-D-Dab-1) and D86(6-L-Dab-1)) containing 6-D-Dab or 6-L-Dab residues at positions 3, 7, 11,18, 22, and 26 (denoted by −1). Thus, these 26-residue peptides containing 6-D-Dab residues consist of 26-D-amino acid residues, while the peptide with 6-L-Dab residues consists of 20 D-amino acids and 6 L-amino acid residues. The percent lysis of the human red blood cells from the four different human blood donors (donors “A, B, C, and D”) is shown in the four panels of FIG. 7A. These results highlight the differences between the two peptides for the four different blood donors. FIG. 7A highlights the differences between the two peptides where the peptide containing 6 L-Dab residues exhibits very little hemolytic activity compared to the peptide containing 6 D-Dab residues in a peptide containing all-D enantiomeric form amino acids. The inventors believe that the 6-L-Dab substitutions in the peptide consisting of 20 D-amino acid residues and 6-L amino acid residues systematically alter the conformation of the peptide at positions 3, 7, 11, 18, 22, and 26 where the L-Dab residues are substituted. FIG. 7B shows the differences between the four blood donors, wherein blood donor B shows the greatest difference between the two peptides.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

What is claimed is:

1. An antimicrobial peptide (AMP) comprising 26 amino acid residues comprising:
i) 2 specificity determinants;
ii) non-naturally occurring, positively charged amino acid residues; and,
iii) a mixture of amino acid residues in the D- and L-enantiomeric form.

2. The AMP of claim 1, wherein the non-naturally occurring, positively charged amino acid residues are selected from L-Diaminobutyric acid (L-Dab) and L-Diaminopropionic acid (L-Dap).

3. The AMP of claim 1 or 2, wherein the specificity determinants are individually selected from the group consisting of D-Lysine (Lys), L-Lys, D-Ornithine (Orn), L-Orn, D-Diaminobutyric acid (Dab), L-Dab, D-Diaminopropionic acid (Dap), and L-Dap.

4. The AMP of any one of claims 1-3, wherein the specificity determinants are located at positions 13 and 16 of the AMP.

5. The AMP of any one of claims 1-4, comprising at least 5 amino acids in the L-enantiomeric form.

6. The AMP of any one of claims 1-5, comprising 5 or 6 amino acids in the L-enantiomeric form.

7. The AMP of any one of claims 1-6, wherein the L-enantiomeric amino acids are located at amino acid positions
3, 7, 11, 18, 22, and 26 or
3, 7, 11, 18, and 22, or
3, 7, 14, 15, 22, and 26 or
3, 7, 14, 15, and 22, of the AMP.

8. The AMP of any one of claims 1-7, wherein amino acid residues L-Dab or L-Dap are located at amino acid positions
3, 7, 11, 18, 22, and 26 or
3, 7, 11, 18, and 22, or
3, 7, 14, 15, 22, and 26 or
3, 7, 14, 15, and 22, of the AMP.

9. An antimicrobial peptide (AMP) comprising the amino acid sequence:
D-Lys-Xaa²-Xaa³-D-Ser-Xaa⁵-Xaa⁶-Xaa⁷-D-Thr-Xaa⁹-D-Ser-Xaa¹¹-D-Ala-Xaa¹³-Xaa¹⁴-Xaa¹⁵-Xaa¹⁶-Xaa¹⁷-Xaa¹⁸-D-Thr-Xaa²⁰-Xaa²¹-Xaa²²-D-Ala-Xaa²⁴-D-Ser-Xaa²⁶ (SEQ ID NO:1)
Wherein:
the D- prefix denotes an amino acid residue in the D-enantiomeric form and the L- prefix denotes an amino acid residue in the L-enantiomeric form; and
Xaa², Xaa⁵, Xaa⁶, Xaa⁹, Xaa¹⁷, Xaa²⁰, Xaa²¹, and Xaa²⁴ are each independently selected from D-Leu (Leucine), D-Ile (Isoleucine), and D-Nle (Norleucine);
Xaa³, Xaa⁷, Xaa¹¹, Xaa¹⁸, and Xaa²² are each independently selected from L-Dab (Diaminobutyric acid), L-Dap (Diaminopropionic acid), D-Dab, D-Dap, D-Orn (Ornithine), D-Lys (Lysine), D-Ala (Alanine), and D-Arg (Arginine);
X¹³ and X¹⁶ are each independently selected from L-Dab, L-Dap, D-Dab, D-Dap, and D-Lys;
X¹⁴ and X¹⁵ are each independently selected from D-Lys, L-Dab, L-Dap, D-Dab, D-Dap, and D-Ala; and,
X²⁶ is selected from L-Dab, L-Dap, D-Dab, D-Dap, D-Cys (Cysteine), D-Ser (Serine), D-Orn, D-Lys, and D-Arg;

10. An AMP of claim 9, wherein the AMP comprises an amino acid sequence selected from the group consisting of:

| | SEQ |
|---|---|
| | ID |
| Sequence | NO |
| KL(L-Dab)SLL(L-Dab)TLS(L-Dab)AKAAKL(L-Dab)TLL | 2 |
| (L-Dab)ALS(L-Dab) | |
| KL(L-Dab)SLL(L-Dab)TLS(L-Dab)AKAAKL(L-Dab)TLL | 3 |
| (L-Dab)ALSC | |
| KL(L-Dap)SLL(L-Dap)TLS(L-Dap)AKAAKL(L-Dap)TLL | 4 |
| (L-Dap)ALSC | |

wherein: the sequence is provided using the one-letter code for all amino acid residues except where the three-letter code is used; all amino acids are in the D-enantiomeric form unless the L-enantiomeric form is indicated by the prefix “L-”; and SEQ ID NO: 2 is optionally covalently linked to a polyethylene glycol (PEG) molecule at the amino terminal lysine (K) residue; and SEQ ID NOs:3 and 4 are optionally covalently linked to a PEG molecule at the carboxy terminal cysteine (C) residue.

11. An AMP of claim 9, wherein the AMP comprises an amino acid sequence selected from the group consisting of:

| Sequence | SEQ ID NO |
|---|---|
| K(Nle)(L-Dab)S(Nle)(Nle)(L-Dab)T(Nle)S(L-Dab)AKAAK(Nle) | 5 |
| (L-Dab)T(Nle)(Nle)(L-Dab)A(Nle)SS | |
| K(Nle)(L-Dap)S(Nle)(Nle)(L-Dap)T(Nle)S(L-Dap)AKAAK(Nle) | 6 |
| (L-Dap)T(Nle)(Nle)(L-Dap)A(Nle)SS | |
| K(Leu)(L-Dab)S(Leu)(Leu)(L-Dab)T(Leu)S(L-Dab)AKAAK(Leu) | 7 |
| (L-Dab)T(Leu)(Leu)(L-Dab)A(Leu)SS | |
| K(Ile)(L-Dab)S(Ile)(Ile)(L-Dab)T(Ile)S(L-Dab)AKAAK(Ile) | 8 |
| (L-Dab)T(Ile)(Ile)(L-Dab)A(Ile)SS | |
| K(Nle)(L-Dab)S(Nle)(Nle)(L-Dab)T(Nle)SAAK(L-Dab)(L-Dab) | 9 |
| K(Nle)AT(Nle)(Nle)(L-Dab)A(Nle)SS | |
| K(Nle)(L-Dap)S(Nle)(Nle)(L-Dap)T(Nle)SAAK(L-Dap)(L-Dap) | 10 |
| K(Nle)AT(Nle)(Nle)(L-Dap)A(Nle)SS | |
| K(Leu)(L-Dab)S(Leu)(Leu)(L-Dab)T(Leu)SAAK(L-Dab)(L-Dab) | 11 |
| K(Leu)AT(Leu)(Leu)(L-Dab)A(Leu)SS | |
| K(Ile)(L-Dab)S(Ile)(Ile)(L-Dab)T(Ile)SAAK(L-Dab)(L-Dab) | 12 |
| K(Ile)AT(Ile)(Ile)(L-Dab)A(Ile)SS | |
| K(Nle)(D-Dab)S(Nle)(Nle)(D-Dab)T(Nle)SAAK(D-Dab)(D-Dab) | 13 |
| K(Nle)AT(Nle)(Nle)(D-Dab)A(Nle)SS | |
| K(Nle)(D-Dap)S(Nle)(Nle)(D-Dap)T(Nle)SAAK(D-Dap)(D-Dap) | 14 |
| K(Nle)AT(Nle)(Nle)(D-Dap)A(Nle)SS | |
| K(Leu)(D-Dab)S(Leu)(Leu)(D-Dab)T(Leu)SAAK(D-Dab)(D-Dab) | 15 |
| K(Leu)AT(Leu)(Leu)(D-Dab)A(Leu)SS | |
| K(Ile)(D-Dab)S(Ile)(Ile)(D-Dab)T(Ile)SAAK(D-Dab)(D-Dab) | 16 |
| K(Ile)AT(Ile)(Ile)(D-Dab)A(Ile)SS | |

wherein: the sequence is provided using the one-letter code for all amino acid residues except where the three-letter code is used; and all amino acids are in the D-enantiomeric form unless the L-enantiomeric form is indicated by the prefix “L-”.

12. An AMP of claim 9, wherein the AMP comprises an amino acid sequence selected from the group consisting of:

| | SEQ |
|---|---|
| Sequence | ID NO |
| KL(L-Dab)SLL(L-Dab)TLS(L-Dab)AKAAKL(L-Dab)TLL(L-Dab)ALSS | 7 |
| KL(D-Dab)SLL(D-Dab)TLS(D-Dab)AKAAKL(D-Dab)TLL(D-Dab)ALSS | 17 |
| KL(L-Dab)SLL(D-Dab)TLS(D-Dab)AKAAKL(D-Dab)TLL(L-Dab)ALSS | 18 |
| KL(D-Dab)SLL(L-Dab)TLS(L-Dab)AKAAKL(L-Dab)TLL(D-Dab)ALSS | 19 |
| KL(L-Dab)SLL(L-Dab)TLSAAK(L-Dab)(L-Dab)KLATLL(L-Dab)ALSS | 11 |
| KL(D-Dab)SLL(D-Dab)TLSAAK(D-Dab)(D-Dab)KLATLL(D-Dab)ALSS | 20 |
| KL(L-Dab)SLL(L-Dab)TLSAAK(D-Dab)(D-Dab)KLATLL(L-Dab)ALSS | 21 |
| KL(D-Dab)SLL(D-Dab)TLSAAK(L-Dab)(L-Dab)KLATLL(D-Dab)ALSS | 22 |
| KL(L-Dap)SLL(L-Dap)TLS(L-Dap)AKAAKL(L-Dap)TLL(L-Dap)ALS(L-Dap) | 23 |
| KL(D-Dap)SLL(D-Dap)TLS(D-Dap)AKAAKL(D-Dap)TLL(D-Dap)ALS(D-Dap) | 24 |
| KL(L-Dap)SLL(L-Dap)TLS(D-Dap)AKAAKL(D-Dap)TLL(L-Dap)ALS(L-Dap) | 25 |
| KL(D-Dap)SLL(D-Dap)TLS(L-Dap)AKAAKL(L-Dap)TLL(D-Dap)ALS(D-Dap) | 26 |
| KL(L-Dap)SLL(L-Dap)TLS(L-Dap)AKAAKL(L-Dap)TLL(L-Dap)ALS(Ser) | 27 |
| KL(D-Dap)SLL(D-Dap)TLS(D-Dap)AKAAKL(D-Dap)TLL(D-Dap)ALS(Ser) | 28 |
| KL(L-Dap)SLL(L-Dap)TLSAAK(L-Dap)(L-Dap)KLATLL(L-Dap)ALS(L-Dap) | 29 |
| KL(D-Dap)SLL(D-Dap)TLSAAK(D-Dap)(D-Dap)KLATLL(D-Dap)ALS(D-Dap) | 30 |
| KL(L-Dap)SLL(L-Dap)TLSAAK(D-Dap)(D-Dap)KLATLL(L-Dap)ALS(L-Dap) | 31 |
| KL(D-Dap)SLL(D-Dap)TLSAAK(L-Dap)(L-Dap)KLATLL(D-Dap)ALS(D-Dap) | 32 |

wherein: the sequence is provided using the one-letter code for all amino acid residues except where the three-letter code is used; and all amino acids are in the D-enantiomeric form unless the L-enantiomeric form is indicated by the prefix “L-”.

13. An AMP of claim 9, wherein the AMP comprises an amino acid sequence selected from the group consisting of:

| | SEQ |
|---|---|
| | ID |
| Sequence | NO |
| KL(L-Dab)SLL(L-Dab)TLSAA(Lys)(L-Dab)(L-Dab) | 33 |
| (Lys)LATLL(L-Dab)ALS(L-Dab) | |
| KL(L-Dab)SLL(L-Dab)TLSAA(L-Dab)(L-Dab)(L-Dab) | 34 |
| (L-Dab)LATLL(L-Dab)ALS (L-Dab) | |
| KL(L-Dab)SLL(L-Dab)TLSAA(L-Dap)(L-Dab)(L-Dab) | 35 |
| (L-Dap)LATLL(L-Dab)ALS(L-Dab) | |
| KL(D-Dab)SLL(D-Dab)TLSAA(D-Dab)(D-Dab)(D-Dab) | 36 |
| (D-Dab)LATLL(D-Dab)ALS(D-Dab) | |
| KL(D-Dab)SLL(D-Dab)TLSAA(D-Dap)(D-Dab)(D-Dab) | 37 |
| (D-Dap)LATLL(D-Dab)ALS(D-Dab) | |

wherein: the sequence is provided using the one-letter code for all amino acid residues except where the three-letter code is used; and all amino acids are in the D-enantiomeric form unless the L-enantiomeric form is indicated by the prefix “L-”.

14. An AMP of claim 9, wherein the AMP comprises an amino acid sequence selected from the group consisting of:

| Sequence | SEQ ID NO |
|---|---|
| KL (Lys) SLL (Lys) TLS | 38 |
| (Lys) AKAAKL (Lys) TLL | |
| (Lys) ALS (Lys) | |
| KL(L-Dab)SLL(L-Dab)TLS | 2 |
| (L-Dab)AKAAKL(L-Dab)TLL | |
| (L-Dab)ALS(L-Dab) | |
| KL(L-Dap)SLL(L-Dap)TLS | 23 |
| (L-Dap)AKAAKL(L-Dap)TLL | |
| (L-Dap)ALS(L-Dap) | |
| KL (Lys) SLL (Lys) TLS | 39 |
| (Lys) AKAAKL (Lys) TLL | |
| (Lys) ALS (Ser) | |
| KL(L-Dab)SLL(L-Dab)TLS | 7 |
| (L-Dab)AKAAKL(L-Dab)TLL | |
| (L-Dab)ALS (Ser) | |
| KL (Lys) SLL (Lys) TLSAA | 40 |
| K (Lys) (Lys) KLATLL | |
| (Lys) ALS (Lys) | |
| KL(L-Dab)SLL(L-Dab)TLSAAK | 41 |
| (L-Dab)(L-Dab) KLATLL | |
| (L-Dab)ALS(L-Dab) | |
| KL(L-Dap)SLL(L-Dap)TLSAAK | 29 |
| (L-Dap)(L-Dap) KLATLL | |
| (L-Dap)ALS(L-Dap) | |
| KL (Lys) SLL (Lys) TLSAA K | 42 |
| (Lys) (Lys) KLATLL (Lys) | |
| ALS (Ser) | |
| KL(L-Dab)SLL(L-Dab)TLSAA K | 11 |
| (L-Dab)(L-Dab) KLATLL(L-Dab) | |
| ALS(Ser) | |

wherein: the sequence is provided using the one-letter code for all amino acid residues except where the three-letter code is used; and all amino acids are in the D-enantiomeric form unless the L-enantiomeric form is indicated by the prefix “L-”.

15. An AMP of claim 9, wherein the AMP comprises an amino acid sequence selected from the group consisting of:

| Sequence | SEQ ID NO |
|---|---|
| KL (Arg) SLL (Arg) TLS (Arg) | 43 |
| AKAAKL (Arg) TLL (Arg) ALS | |
| (Arg) | |
| KL (Lys) SLL (Lys) TLS (Lys) | 38 |
| AKAAKL (Lys) TLL (Lys) ALS | |
| (Lys) | |
| KL (Orn) SLL (Orn) TLS (Orn) | 44 |
| AKAAKL (Orn) TLL (Orn) ALS | |
| (Orn) | |
| KL (L-Dab) SLL (L-Dab) TLS | 2 |
| (L-Dab)AKAAKL (L-Dab) TLL | |
| (L-Dab) ALS (L-Dab) | |
| KL (L-Dap) SLL (L-Dap) TLS | 23 |
| (L-Dap)AKAAKL (L-Dap) TLL | |
| (L-Dap) ALS (L-Dap) | |
| KL (Lys) SLL (Lys) TLS (Lys) | 39 |
| AKAAKL (Lys) TLL (Lys) ALSS | |
| KL (L-Dab) SLL (L-Dab) TLS | 7 |
| (L-Dab)AKAAKL (L-Dab) TLL | |
| (L-Dab)ALSS | |

wherein: the sequence is provided using the one-letter code for all amino acid residues except where the three-letter code is used; and all amino acids are in the D-enantiomeric form unless the L-enantiomeric form is indicated by the prefix “L-”.

16. The AMP of any one of claims 1-15, which is covalently linked to a polyethylene glycol (PEG) molecule.

17. The AMP of any one of claims 1-16, which is covalently linked to one or more domains of an Fc region of human IgG immunogloblin to at least one of the amino-terminus or carboxyl-terminus of the peptide.

18. The AMP of any one of claims 1-17, wherein the AMP inhibits propagation of a prokaryote.

19. The AMP of claim 18, wherein the prokaryote is a Gram-negative bacterium.

20. The AMP of claim 19, wherein the Gram-negative bacterium is at least one of A. baumannii and P. aeruginosa.

21. The AMP of any one of claims 9-20, wherein the therapeutic index (calculated by the ratio of hemolytic activity and antimicrobial activity (MIC)) is at least 100.

22. The AMP of any one of claims 9-20, wherein the therapeutic index (calculated by the ratio of hemolytic activity and antimicrobial activity (MIC)) is between 100 and 1100.

23. The AMP of any one of claims 9-20, wherein the therapeutic index (calculated by the ratio of hemolytic activity and antimicrobial activity (MIC)) is between 700 and 1100.

24. The AMP of any one of claims 9-20, wherein the therapeutic index (calculated by the ratio of hemolytic activity and antimicrobial activity (MIC)) is between 950 and 1100.

25. The AMP of any one of claims 9-24, wherein the AMP exhibits greater antimicrobial activity against Gram-negative P. aeruginosa or Acinetobacter baumannii drug-resistant mutants compared to other AMPs.

26. The AMP of any one of claims 9-25, wherein the AMP exhibits at least about a 20-fold greater antimicrobial activity against Gram-negative Acinetobacter baumannii bacteria compared to Polymyxin B.

27. The AMP of any one of claims 9-26, wherein the AMP exhibits at least a 13-fold decrease in hemolysis of human red blood cells compared to hemolysis exhibited by SEQ ID NO:43.

28. A pharmaceutical composition comprising at least one AMP of any one of claims 9-27 and a pharmaceutically acceptable carrier.

29. The pharmaceutical composition of claim 28, comprising a mono-phasic pharmaceutical composition suitable for parenteral or oral administration consisting essentially of a therapeutically-effective amount of the at least one peptide, and a pharmaceutically acceptable carrier.

30. A method of preventing or treating a microbial infection comprising administering to a subject in need thereof a therapeutically effective amount of at least one AMP of any one of claims 9-26, or a pharmaceutical composition of claim 28 or 29.

31. The method of claim 30, wherein the microbial infection is a bacterial infection.

32. The method of claim 31, wherein the bacterial infection is a Gram-negative bacterial infection.

33. The method of claim 32, wherein the bacterial infection is an antibiotic-resistant Gram-negative bacterial infection.

34. The method of claim 31, wherein the infecting microorganism is at least one of Acinetobacter baumannii and Pseudomonas aeruginosa.

35. The method of claim 31, wherein an infecting microorganism is multi-drug resistant Pseudomonas aeruginosa or Acinetobacter baumannii.

36. The method of any one of claims 30-35, wherein the administration of the peptide or pharmaceutical composition is by an administration route selected from oral, topical, subcutaneous, intravenous, intraperitoneal, intramuscular, intradermal, intrasternal, intraarticular injection, intrathecal, and infusion.

37. The method of claim 36, wherein the peptide or pharmaceutical composition is administered in conjunction with one or more additional antimicrobial agents.

38. A method of preventing a microbial infection in an individual at risk of developing an infection comprising administering an effective amount of at least one peptide of any one of claims 9-27, or a pharmaceutical composition of claim 28 or 29.

39. The method of claim 38, wherein the individual is a surgical patient.

40. The method of claim 38, wherein the individual is a hospitalized patient.

41. A method of treating a topical bacterial infection in a patient, comprising applying at least one peptide of any one of any one of claims 9-27, or a pharmaceutical composition of claim 28 or 29, to a body surface of the patient.

42. The method of claim 41, wherein the body surface is a wound.

43. The method of claim 42, wherein the composition is applied following an operation or surgery.

44. At least one peptide of any one of claims 9-26, or a pharmaceutical composition of claim 28 or 29 for use in the treatment of a microbial infection.

45. Use of any one of claims 9-27, or a pharmaceutical composition of claim 28 or 29, in the manufacture of a medicament for the prevention or treatment of a microbial infection.
